(19) United States
(12) Patent Application Publication
(10) Pub. No.: US 2024/0026031 A1
(43) Pub. Date: Jan. 25, 2024

(54) DEGRADATION OF SURFACE PROTEINS USING BISPECIFIC BINDING AGENT

(71) Applicant: The Regents of the University of California, Oakland, CA (US)

(72) Inventors: James A. WELLS, Oakland, CA (US); Adam D. COTTON, Oakland, CA (US); Duy NGUYEN, Oakland, CA (US); Katarina PANCE, Oakland, CA (US)

(21) Appl. No.: 17/773,283

(22) PCT Filed: Oct. 30, 2020
(86) PCT No.: PCT/US2020/058328

§ 371 Date: Apr. 29, 2022

Related U.S. Application Data
(60) Provisional application No. 62/929,674, filed on Nov. 1, 2019.

(51) Int. Cl. C07K 16/40; C07K 16/28; A61K 47/68

(52) U.S. Cl. CPC C07K 16/40; C07K 16/2827; A61K 47/6879; A61K 47/6803; C07K 2317/55; C07K 2317/31; C07K 2317/622; C07K 2317/92

(57) ABSTRACT

The present disclosure relates to, among other things, methods for degrading targeted surface proteins using the ubiquitin pathway by using a bispecific binding agent or an immunoconjugate that binds the targeted surface protein and a membrane-associated ubiquitin E3 ligase. The present disclosure also relates to methods for degrading targeted surface proteins using the ubiquitin pathway using an engineered transmembrane protein that binds the targeted surface protein and exhibits ubiquitin E3 ligase activity. The disclosure also provides compositions and methods useful for producing such bispecific binding agents and engineered transmembrane proteins, immunoconjugates, nucleic acids encoding same, host cells genetically modified with the nucleic acids, as well as methods for modulating an activity of a cell and/or for the treatment of various diseases such as cancers.

### CROSS-REFERENCES TO RELATED APPLICATIONS

This application claims the benefit of U.S. Provisional Application No. 62/929,674, filed Nov. 1, 2019, which is incorporated herein by reference in entirety and for all purposes.

### STATEMENT REGARDING FEDERALLY SPONSORED R&D

This invention was made with government support under grants P41 CA196276 and R35 GM122451 awarded by The National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 048536-670001WO_SequenceListing_ST25, created Oct. 30, 2020, 213,455 bytes, machine format IBM-PC, MS Windows operating system, is hereby incorporated by reference.

### FIELD

The present disclosure relates generally to new methods and agents for degrading surface proteins on a cell using the ubiquitin pathway. The disclosure also provides methods useful for producing such agents, nucleic acids encoding same, host cells genetically modified with the nucleic acids, as well as methods for modulating an activity of a cell and/or for the treatment of various diseases such as cancers.

### BACKGROUND

Malignant neoplastic cells often express surface proteins that have proliferative or immunosuppressive effects. For example, some tumors overexpress a growth factor receptor (such as HER2 or HER3) which stimulates proliferation. Overexpression of immune checkpoint proteins (such as PD-L1 and CTLA-4) can suppress the native immune response. This allows malignant cells to multiply and evade the host immune system, leading to tumor formation.

Current therapies address the problem using antibodies and antibody derivatives that specifically bind these surface proteins and inhibit their activity while bound. However, there is a need for therapies that have a better effect (e.g., more durable effect) and/or that target cancer cells or neoplastics cells by either increasing the effect of responses that fight the cancer and/or counteracting surface proteins that have proliferative or immunosuppressive effects. The disclosure provided herein addresses these problems and provides additional solutions as well.

All references and patents cited herein are hereby incorporated by reference in full, as if fully set forth herein.

### SUMMARY

The present disclosure describes new therapeutic methods and agents that promote the removal and degradation of targeted surface proteins using the ubiquitin pathway. Described herein are bispecific binding agents that bind both a target surface protein and a membrane-associated ubiquitin E3 ligase, wherein binding of the bispecific binding agent leads to ubiquitination of the target surface protein and its subsequent degradation. Also described herein are E3 ligase derivatives having a target surface protein binding domain, which result in ubiquitination of the target surface protein when the E3 ligase derivative is present in the target cell's plasma membrane.

An aspect of the disclosure is a bispecific binding agent comprising a first binding domain that specifically binds to an E3 ligase; and a second binding domain that specifically binds to an extracellular epitope on a target protein of a target cell, wherein both the E3 ligase and the target protein are membrane associated. An embodiment is the bispecific binding agent wherein binding of the bispecific binding agent to both the E3 ligase and the target protein results in ubiquitination of the target protein. An embodiment is the bispecific binding agent wherein the target cell is a neoplastic cell. An embodiment is the bispecific binding agent wherein the cell is a cancer cell selected from the group consisting of breast cancer, B cell lymphoma, pancreatic cancer, Hodgkin's lymphoma, ovarian cancer, prostate cancer, mesothelioma, lung cancer, non-Hodgkin's B-cell (B-NHL), melanoma, chronic lymphocytic leukemia, acute lymphocytic leukemia, neuroblastoma, glioma, glioblastoma, bladder cancer, and colorectal cancer. An embodiment is the bispecific binding agent wherein the target protein is an immune checkpoint protein. An embodiment is the bispecific binding agent wherein the target protein is selected from the group consisting of PD-L1, PD-1, CTLA-4, A2AR, B7-H3, B7-H4, BTLA, KIR, LAG3, NKG2D, TIM-3, VISTA, and SIGLEC7. In some embodiments, the first binding domain of the bispecific binding agent specifically binds to an extracellular protein attached to an E3 ligase. In some embodiments, the first binding domain of the bispecific binding agent specifically binds to a transmembrane protein that interacts with an E3 ligase. In certain embodiments of the bispecific binding agent wherein degradation of the target protein reduces the ability of the target cell to proliferate. Some protein, for example, A2aR, would modulate the immune system, i.e., it would boost CD8 immune response and proliferation. In certain embodiments of the bispecific binding agent wherein the target protein is selected from the group consisting of HER2, CD19, CD20, PD-L1, EGFR, CTLA-4, MMP14, and CDCP1.

In other embodiments, the E3 ligase of the bispecific binding agent comprises a transmembrane protein. For instance, in some embodiments, the E3 ligase comprises a transmembrane E3 ligase. In some exemplary embodiments, the E3 ligase is selected from the group consisting of RNF43, RNF128 (GRAIL), ZNRF3, and MARCH11. An embodiment is the bispecific binding agent wherein the first binding domain and the second binding domain are each independently selected from the group consisting of half antibodies, single-domain antibodies, nanobodies, monospecific Fab₂, scFv, scFv-Fc, minibodies, IgNAR, V-NAR, hcIgG, VhH, camelid antibodies, and peptibodies, or the first binding domain and the second binding domain together form a bispecific antibody, a bispecific diabody, a bispecific Fab₂, a bispecific camelid antibody, or a bispecific peptibody. An embodiment is the bispecific binding agent wherein the bispecific binding agent comprises a bispecific antibody. An embodiment is the bispecific binding agent wherein the bispecific binding agent comprises a bispecific IgG. An embodiment is the bispecific binding agent wherein the bispecific binding agent comprises a knob and hole bispecific IgG. An embodiment is the bispecific binding agent wherein the first binding domain comprises a Fab, and the second binding domain comprises a single chain Fab. An embodiment is the bispecific binding agent wherein the first binding domain comprises a Fab, and the second binding domain comprises an scFv. In some embodiments, the first binding domain comprises heavy chain framework region (FR) sequence set forth in SEQ ID NOs.: 12 or 320 and light chain FR sequence set forth in SEQ ID NOs.: 11 or 319. In some embodiments, the second binding domain comprises heavy chain FR sequence set forth in SEQ ID NOs.: 12 or 320 and light chain FR sequence set forth in SEQ ID NOs.: 11 or 319.

In some embodiments, the first binding domain comprises light chain variable domain CDR3 (LC-CDR3) sequence and heavy chain variable domain CDR1 (HC-CDR1), HC-CDR2, and HC-CDR3 sequences comprising the sequences set forth in Table 2, respectively. In some embodiments, the second binding domain comprises LC-CDR3 sequence and HC-CDR1, HC-CDR2, and HC-CDR3 sequences comprising the sequences set forth in Table 3, respectively.

In some embodiments, the first binding domain of the bispecific binding agent comprises a heavy chain variable domain (VH), and wherein the VH comprises the FR sequence set forth in SEQ ID NO.: 321; and the second binding domain of the bispecific binding agent comprises a heavy chain FR sequence set forth in SEQ ID NOs.: 12 or 320 and light chain FR sequence set forth in SEQ ID NOs.: 11 or 319.

In some embodiments, the first binding domain comprises VH-CDR1, VH-CDR2, and VH-CDR3 sequences set forth in Table 4, respectively, and the second binding domain comprises LC-CDR3 sequence and HC-CDR1, HC-CDR2, and HC-CDR3 sequences comprising the sequences set forth in Table 3, respectively.

An aspect of the disclosure is a nucleic acid that encodes any one of the bispecific binding agents comprising a first binding domain that specifically binds to a E3 ligase; and a second binding domain that specifically binds to an extracellular epitope on a target protein of a target cell, wherein both the E3 ligase and the target protein are membrane associated. An embodiment is the nucleic acid that further comprises a vector. An embodiment is the nucleic acid that further comprises a promoter operably linked to the bispecific binding agent encoding sequence.

An aspect of the disclosure is a vector comprising a nucleic acid that encodes any one of the bispecific binding agents described above or set forth herein. An embodiment is the vector further comprising a promoter operably linked to the bispecific binding agent encoding sequence.

An aspect of the disclosure is an immunoconjugate. In some embodiments, the immunoconjugate comprises a bispecific binding agent disclosed herein and a small molecule. In other embodiments, the immunoconjugate further comprises a linker. In certain embodiments, the linker is selected from the group consisting of a cleavable linker, a non-cleavable linker, a hydrophilic linker, and a dicarboxylic acid based linker. In some embodiments, the immunoconjugate comprises DBCO-PEG4-CGS21680, wherein the DBCO is used for conjugation, the PEG4 is the linker, and the CGS21680 is the small molecule. In some embodiments, the immunoconjugate comprises DBCO-PEG4-amine. In some embodiments, the small molecule comprises amine, CGS21680, oxaziridine-azide, ZM241385, plerixafor, maraviroc, and aplaviroc. However, it is understood that the small molecule can be any small molecule one skilled in the art deems suitable.

An aspect of the disclosure is a pharmaceutical composition comprising a pharmaceutically acceptable carrier, and a bispecific binding agent set forth herein, an immunoconjugate of the disclosure; or a nucleic acid set forth herein. An embodiment is the pharmaceutical composition wherein binding of the bispecific binding agent to both the E3 ligase and the target protein results in ubiquitination of the target protein. An embodiment is the pharmaceutical composition wherein the target cell is a neoplastic cell. An embodiment is the pharmaceutical composition wherein the cell is a cancer cell selected from the group consisting of breast cancer, B cell lymphoma, pancreatic cancer, Hodgkin's lymphoma, ovarian cancer, prostate cancer, mesothelioma, lung cancer, non-Hodgkin's B-cell (B-NHL), melanoma, chronic lymphocytic leukemia, acute lymphocytic leukemia, neuroblastoma, glioma, glioblastoma, bladder cancer, and colorectal cancer. An embodiment is the pharmaceutical composition wherein the target protein is an immune checkpoint protein. An embodiment is the pharmaceutical composition wherein the target protein is selected from the group consisting of PD-L1, PD-1, CTLA-4, A2AR, B7-H3, B7-H4, BTLA, KIR, LAG3, NKG2D, TIM-3, VISTA, and SIGLEC7. An embodiment is the pharmaceutical composition wherein degradation of the target protein reduces the ability of the target cell to proliferate. An embodiment is the pharmaceutical composition wherein the target protein is selected from the group consisting of HER2, CD19, CD20, PD-L1, EGFR, CTLA-4, MMP14, and CDCP1.

An aspect of the disclosure is an engineered cell, comprising a cell capable of protein expression, and a nucleic acid that encodes a bispecific binding agent. An embodiment is the engineered cell wherein the cell is a B cell, a B memory cell, or a plasma cell.

An aspect of the disclosure is a method of treating a neoplastic disease or disorder in a subject, the method comprising administering to a subject in need thereof, a therapeutically effective amount of bispecific binding agent of the disclosure, a nucleic acid of the disclosure, a pharmaceutical composition of the disclosure, or an engineered cell of the disclosure.

An aspect of the disclosure is a method of making the bispecific binding agent of the disclosure, by providing a cell capable of protein synthesis that comprises a nucleic acid that encodes a bispecific binding agent of the disclosure, and inducing expression of the bispecific binding agent.

An aspect of the disclosure is an engineered transmembrane protein for the treatment of neoplastic disease in which a target protein is present on the surface of a neoplastic cell or an immune cell, comprising a membrane-associated E3 ligase linked to a target protein binding domain specific for the target protein. An embodiment is the engineered transmembrane protein wherein the E3 ligase and the target protein binding domain are covalently linked. An embodiment is the engineered transmembrane protein wherein the E3 ligase and the target protein binding domain are expressed as a fusion protein. An embodiment is the engineered transmembrane protein wherein the E3 ligase and the target protein binding domain are covalently linked by a disulfide bond. An embodiment is the engineered transmembrane protein wherein the target protein binding domain is specific for a target protein is selected from the group consisting of HER2, EGFR, MMP14, CDCP1, PD-L1, PD-1, CTLA-4, CD19, CD20, A2AR, B7-H3, B7-H4, BTLA, KIR, LAG3, NKG2D, TIM-3, VISTA, and SIGLEC7.

An aspect of the disclosure is a nucleic acid that encodes the engineered transmembrane protein of the disclosure. An embodiment is the nucleic acid that further comprises a vector. An embodiment is the nucleic acid that further comprises a promoter operably linked to the sequence encoding the engineered transmembrane protein.

An aspect of the disclosure is a composition for the treatment of a neoplastic disease in which a target protein is present on the surface of a neoplastic cell, the composition comprising a therapeutic amount of the engineered transmembrane protein of the disclosure, and a fusogenic carrier, wherein the carrier is capable of fusing with the neoplastic cell plasma membrane. An embodiment is the composition wherein the carrier is a fusogenic liposome.

An aspect of the disclosure is a composition for the treatment of a neoplastic disease in which a target protein is present on the surface of a neoplastic cell, the composition comprising a therapeutic amount of a nucleic acid encoding the engineered transmembrane protein of the disclosure, and a pharmaceutically acceptable carrier, wherein the carrier is capable of delivering the nucleic acid to the neoplastic cell cytosol. An embodiment is the composition wherein the carrier comprises a viral particle, a liposome, or an exosome. An embodiment is the composition wherein the carrier comprises a viral particle. An embodiment is the composition wherein the carrier comprises a liposome. An embodiment is the composition wherein the carrier comprises an exosome.

An aspect of the disclosure is the use for the treatment of a neoplastic disease of: a bispecific binding agent of the disclosure; a nucleic acid encoding a bispecific binding agent of the disclosure; an engineered transmembrane protein of the disclosure; a nucleic acid encoding an engineered transmembrane protein of the disclosure; a pharmaceutical composition comprising a bispecific binding agent of the disclosure, a nucleic acid encoding a bispecific binding agent of the disclosure, an immunoconjugate of the disclosure; an engineered transmembrane protein of the disclosure, or a nucleic acid encoding an engineered transmembrane protein of the disclosure; a vector encoding a bispecific binding agent of the disclosure or an engineered transmembrane protein of the disclosure; or an engineered cell comprising a nucleic acid encoding a bispecific binding agent of the disclosure.

An aspect of the disclosure is the use manufacture of a medicament for the treatment of a neoplastic disease of: a bispecific binding agent of the disclosure; a nucleic acid encoding a bispecific binding agent of the disclosure; an immunoconjugate of the disclosure; an engineered transmembrane protein of the disclosure; a nucleic acid encoding an engineered transmembrane protein of the disclosure; a pharmaceutical composition comprising a bispecific binding agent of the disclosure, a nucleic acid encoding a bispecific binding agent of the disclosure, an engineered transmembrane protein of the disclosure, or a nucleic acid encoding an engineered transmembrane protein of the disclosure; a vector encoding a bispecific binding agent of the disclosure or an engineered transmembrane protein of the disclosure; or an engineered cell comprising a nucleic acid encoding a bispecific binding agent of the disclosure.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIG. 1 schematically depicts a bispecific binding agent of the disclosure (here, an exemplary bispecific antibody), bound to a membrane-associated E3 ligase RNF43 and a target surface protein of interest (“POI”), and the intracellular ubiquitination of the POI.

FIG. 2 schematically depicts an engineered transmembrane protein having a GFP binding domain and a membrane-associated E3 ligase domain, and a membrane bound reporter construct comprising an intracellular NanoLuc domain and an extracellular GFP domain. Binding of the GFP domain by the engineered transmembrane protein promotes intracellular ubiquitination of the NanoLuc domain, resulting in its degradation and loss of signal.

FIG. 3 schematically depicts a bispecific IgG antibody of the disclosure having a “knob-into-hole” configuration.

FIG. 4A shows the results of an experiment using a bispecific IgG of the disclosure to remove and degrade PD-L1 from a triple negative breast cancer cell line (MDA-MB-231). FIG. 4A shows that PD-L1 levels are not affected by an anti-RNF43 antibody (R3 IgG) or an anti-PD-L1 antibody (Tecentriq®), but are substantially reduced or eliminated by using 10 nM of the bispecific anti-RNF43/PD-L1 IgG of the disclosure for 24 hours. FIG. 4B shows a dose response using the same cells and bispecific IgG, showing that maximal PD-L1 degradation is achieved with 10 nM bispecific IgG.

FIG. 5A, FIG. 5B, and FIG. 5C compare the PD-L1 degradation activity of a bispecific IgG of the disclosure with Tecentriq® (atezolizumab) on three different cancer cell lines. Both agents are applied at 10 nM for 24 hours. FIG. 5A shows that the bispecific IgG substantially degraded PD-L1 in MDA-MB-231 cells (a model for triple-negative breast cancer), whereas atezolizumab did not promote degradation or down-regulation of PD-L1 expression. FIG. 5B shows that the bispecific IgG substantially degraded PD-L1 in HCC827 cells (a model for non-small cell lung cancer), whereas atezolizumab did not result in degradation or down-regulation of PD-L1 expression. FIG. 5C shows that the bispecific IgG substantially degraded PD-L1 in T24 cells (a model for advanced bladder cancer), whereas atezolizumab did not promote substantial degradation or down-regulation of PD-L1 expression.

FIG. 6 is a bar graph showing the effects of bispecific IgGs of the disclosure on degrading PD-L1 from a triple negative breast cancer cell line (MDA-MB-231).

FIG. 7 shows a combined bio-layer interferometry (BLI) graphs of each Ala mutant.

FIG. 8 shows the correlation between percent degradation vs Koff.

FIG. 9 shows the correlation between percent degradation vs Kd.

FIG. 10 shows the correlation between percent degradation vs Kon.

FIG. 11 shows the Western blot of anti-RNF43 Alanine mutants. The mutants are labelled by their Kd's to RNF43. 12.5 nM is the WT, 40 nM is S113A and 125 nM is F115A.

FIG. 12 shows a schematic illustration of an immunoconjugate bound to a membrane-associated E3 ligase and a protein of interest (POI).

FIG. 13 is an exemplary schematic illustration of conjugation procedure to generate the immunoconjugate.

FIG. 14 shows some exemplary small molecules used in the present disclosure.

FIG. 15 shows dose dependent degradation of adenosine 2a receptor (A2aR) in MOLT-4 CCR5+ cells after 24 hr treatment of an immunoconjugate degrader.

FIG. 16 shows A2aR levels after 24 hr treatment of CGS21680 (agonist) in MOLT-4 CCR5+ cells.

### DETAILED DESCRIPTION OF THE DISCLOSURE

The present disclosure generally relates to binding agents, including bispecific binding agents and engineered transmembrane proteins, and immunoconjugates thereof, which bind to both a membrane-associated ubiquitin E3 ligase and to a target surface protein present on the surface of a target cell. In some embodiments, the present disclosure provides bispecific binding agents which bind to both a membrane-associated ubiquitin E3 ligase and to a target surface protein present on the surface of a target cell. In other embodiments, the present disclosure provides engineered transmembrane proteins based on modified membrane-associated E3 ligases, having a target surface protein binding domain.

In some embodiments, the present disclosure provides exemplary methods to generate each of the certain types of constructs, such as bispecific IgG, bispecific IgG with a single chain Fab on one arm, and a Fab-scFV fusion. The present disclosure provides methods to test the bispecific IgG, bispecific IgG with a single chain Fab on one arm, and a Fab-scFV fusion. In some embodiments, the present disclosure demonstrates that the bispecific binding agents of the present disclosure are able to degrade their targets in various clinically relevant cell lines.

In some embodiments, the present disclosure provides the synthesis and test of an engineered transmembrane protein in degrading a target protein. In certain embodiments, the present disclosure demonstrates that the engineered transmembrane protein provided herein can cause the internalization and lysosomal aggregation of the target protein. Thus, the present disclosure demonstrates that that the engineered transmembrane protein provided herein can be used to induce protein degradation of endogenous proteins. In some embodiments, the present disclosure further provides methods of generating an AAV transfection vector for inserting an engineered transmembrane protein into a target cell.

In some embodiments, the present disclosure demonstrates that a strong binding affinity between the binding agents provided and their targets can be advantageous. Also provided herein are immunoconjugates comprising the bispecific binding agents and engineered transmembrane proteins of the present disclosure. In some embodiments, the present disclosure demonstrates that an immunoconjugate comprising a binding agent of the present disclosure can be recruited to the target and induce its degradation.

The disclosure also provides nucleic acids that encode the bispecific binding agents or engineered transmembrane proteins, and therapeutic compositions comprising the bispecific binding agents, engineered transmembrane proteins, and/or nucleic acids encoding the bispecific binding agents or engineered transmembrane proteins, and cells comprising the nucleic acid. The disclosure also provides methods of treatment using bispecific binding agents or engineered transmembrane proteins, immunoconjugates, nucleic acids encoding bispecific binding agents or engineered transmembrane proteins, or therapeutic compositions comprising the bispecific binding agents, engineered transmembrane proteins, immunoconjugates, and/or nucleic acids encoding the bispecific binding agents or engineered transmembrane proteins. The disclosure also provides compositions and methods useful for producing such agents, nucleic acids encoding same, host cells genetically modified with the nucleic acids, as well as methods for modulating an activity of a cell and/or for the treatment of various diseases such as cancers.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

### Definitions

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B.”

The terms “administration” and “administering”, as used interchangeably herein, refer to the delivery of a composition or formulation by an administration route including, but not limited to, intravenous, intra-arterial, intracerebral, intrathecal, intramuscular, intraperitoneal, subcutaneous, intramuscular, and combinations thereof. The term includes, but is not limited to, administration by a medical professional and self-administration.

The terms “host cell” and “recombinant cell” are used interchangeably herein. It is understood that such terms, as well as “cell culture”, “cell line”, refer not only to the particular subject cell or cell line but also to the progeny or potential progeny of such a cell or cell line, without regard to the number of transfers. It should be understood that not all progeny are exactly identical to the parental cell. This is because certain modifications may occur in succeeding generations due to either mutation (e.g., deliberate or inadvertent mutations) or environmental influences, such progeny may not, in fact, be identical to the parent cell, but are still included within the scope of the term as used herein, so long as the progeny retain the same functionality as that of the original cell or cell line.

The term “operably linked”, as used herein, denotes a physical or functional linkage between two or more elements, e.g., polypeptide sequences or polynucleotide sequences, which permits them to operate in their intended fashion.

The term “heterologous”, refers to nucleic acid sequences or amino acid sequences operably linked or otherwise joined to one another in a nucleic acid construct or chimeric polypeptide that are not operably linked or are not contiguous to each other in nature.

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See, e.g., the NCBI web site at ncbi.nlm.nih.gov/BLAST. This definition also refers to, or may be applied to, the complement of a test sequence. This definition also includes sequences that have deletions and/or additions, as well as those that have substitutions. Sequence identity typically is calculated over a region that is at least about 20 amino acids or nucleotides in length, or over a region that is 10-100 amino acids or nucleotides in length, or over the entire length of a given sequence. Sequence identity can be calculated using published techniques and widely available computer programs, such as the GCS program package (Devereux et al, Nucleic Acids Res (1984) 12:387), BLASTP, BLASTN, FASTA (Atschul et al., J Mol Biol (1990) 215:403). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

The term “treatment” used in reference to a disease or condition means that at least an amelioration of the symptoms associated with the condition afflicting an individual is achieved, where amelioration is used in a broad sense to refer to at least a reduction in the magnitude of a parameter, e.g., a symptom, associated with the condition being treated. Treatment also includes situations where the pathological condition, or at least symptoms associated therewith, are completely inhibited, e.g., prevented from happening, or eliminated entirely such that the host no longer suffers from the condition, or at least the symptoms that characterize the condition. Thus, treatment includes: (i) prevention (i.e., reducing the risk of development of clinical symptoms, including causing the clinical symptoms not to develop, e.g., preventing disease progression), and (ii) inhibition (i.e., arresting the development or further development of clinical symptoms, e.g., mitigating or completely inhibiting an active disease).

As used herein, and unless otherwise specified, a “therapeutically effective amount” of an agent is an amount sufficient to provide a therapeutic benefit in the treatment or management of the cancer, or to delay or minimize one or more symptoms associated with the cancer. A therapeutically effective amount of a compound means an amount of therapeutic agent, alone or in combination with other therapeutic agents, which provides a therapeutic benefit in the treatment or management of the cancer. The term “therapeutically effective amount” can encompass an amount that improves overall therapy, reduces or avoids symptoms or causes of the cancer, or enhances the therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 2010); Lloyd, The Art, Science and Technology of Pharmaceutical Compounding (2016); Pickar, Dosage Calculations (2012); and Remington: The Science and Practice of Pharmacy, 22nd Edition, 2012, Gennaro, Ed., Lippincott, Williams & Wilkins).

As used herein, a “subject” or an “individual” includes animals, such as human (e.g., human individuals) and non-human animals. In some embodiments, a “subject” or “individual” can be a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, and non-mammals, such as non-human primates, sheep, dogs, cows, chickens, amphibians, reptiles, and the like.

The terms “derivative”, “functional fragment thereof” or “functional variant thereof” refer to a molecule having biological activity in common with the wild-type molecule from which the fragment or derivative was derived. A functional fragment or a functional variant of an antibody is one which retains essentially the same ability to bind to the same epitope as the antibody from which the functional fragment or functional variant was derived. For example, an antibody capable of binding to an epitope of a cell surface receptor may be truncated at the N-terminus and/or C-terminus, and the retention of its epitope binding activity assessed using assays known to those of skill in the art. An antibody derivative may further include constructs based on the general binding properties of antibodies in general, without being directly similar to an existing antibody. For example, one can screen appropriate phage-based libraries for binding to a desired target to obtain binding agents such as nanobodies and scFv agents that are not based on an existing antibody.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

All ranges disclosed herein also encompass any and all possible sub-ranges and combinations of sub-ranges thereof Δny listed range can be recognized as sufficiently describing and enabling the same range being broken down into at least equal halves, thirds, quarters, fifths, tenths, and so forth. As a non-limiting example, each range discussed herein can be readily broken down into a lower third, middle third and upper third, and so forth. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

Although features of the disclosures may be described in the context of a single embodiment, the features may also be provided separately or in any suitable combination. Conversely, although the disclosures may be described herein in the context of separate embodiments for clarity, the disclosures may also be implemented in a single embodiment. Any published patent applications and any other published references, documents, manuscripts, and scientific literature cited herein are incorporated herein by reference for any purpose. In the case of conflict, the present specification, including definitions, will control. In addition, the materials, methods, and examples are illustrative only and not intended to be limiting.

### Ubiquitin

Major pathways of protein degradation in eukaryotic cells involve ubiquitination that targets cellular proteins for rapid proteolysis. Ubiquitination is a highly regulated post-translational process that occurs via covalent transfer of ubiquitin to lysine residues of target proteins. The attachment of ubiquitin is mediated by the cooperative action of three classes of enzymes: ubiquitin-activating enzymes (E1), ubiquitin-conjugating enzymes (E2), and ubiquitin-protein ligases (E3). The ubiquitin-activating enzyme E1 activates ubiquitin in an ATP-dependent process to form a thioester linkage between the C-terminal glycine of ubiquitin and a cysteine residue at the E1 active site. The activated ubiquitin is then transferred to a cysteine residue of the ubiquitin-conjugating enzyme E2. The ubiquitin-protein ligase E3 subsequently promotes the transfer of ubiquitin from the E2 enzyme to the lysine residues of protein substrates. Since the human genome encodes two E1 enzymes, about 40 E2 enzymes, and more than 800 E3 ligases, E3 ligases are primarily responsible for conferring substrate specificity in the protein degradation process. Manipulating the substrate specificity of E3 ligases therefore provides a method to redirect the cellular degradation machinery for the targeted proteolysis of proteins of interest.

### Compositions of the Disclosure

As described in greater detail below, the present disclosure provides binding agents, such as the bispecific binding agents and the engineered transmembrane proteins provided herein, that are useful for degrading a target surface protein present on the surface of a target cell. Without being bound by any particular theory, these agents are designed to function by binding both a target surface protein and a membrane-associated E3 ligase, such that the target surface protein is ubiquitinated and degraded as a result of binding. Also disclosed are engineered transmembrane proteins having a membrane-associated E3 ligase domain and a target surface protein binding domain. Without being bound by any particular theory, these agents are designed to function by binding a target surface protein, such that the target surface protein is ubiquitinated by the membrane-associated E3 ligase domain and is degraded as a result of the binding.

As described in the Examples herein, bispecific binding agents and engineered transmembrane proteins have been tested and validated in tumor cell lines. Without being bound to any particular theory, it is contemplated that these new agents show similar performance in mouse models and in other mammalian cells, as well as in mammalian subjects, including humans. The agents disclosed herein may be introduced into various cell types to create engineered cells for enhanced discrimination and elimination of tumors. Accordingly, engineered cells engineered to express one of more of the agents disclosed herein, are also within the scope of the disclosure.

### Bispecific Binding Agents

### Structure

The bispecific binding agents of the disclosure contain two binding domains: one specific for a membrane-associated E3 ligase, the other specific for a target surface protein. Bispecific binding agents of the disclosure include, without limitation, agents wherein the E3 ligase binding domain and the target surface protein binding domain are each independently selected from an antibody (or half of an antibody), a nanobody, or a minibody, a Fab fragment, a single chain variable fragment (scFv), and a single domain antibody (sdAb), or a functional fragment thereof. These two binding domains can be the same type of molecule, or different. For example, bispecific binding agents of the disclosure include, without limitation, bispecific binding agents having an IgG that binds E3 ligase, and an scFv domain that binds the target surface protein. The two binding domains of the bispecific binding agent can be connected through covalent bonds, non-covalent interactions, or a combination thereof.

The bispecific binding agent can generally take the form of a protein, glycoprotein, lipoprotein, phosphoprotein, and the like. Some bispecific binding agent of the disclosure take the form of bispecific antibodies or antibody derivatives. In some embodiments, the target protein binding domain is selected from the group consisting of a half antibody, a nanobody, or a minibody, a F(ab′)₂ fragment, a Fab fragment, a single chain variable fragment (scFv), and a single domain antibody (sdAb), or a functional fragment thereof. The two binding domains may together take the form of a bispecific antibody, a bispecific diabody, a bispecific camelid antibody or a bispecific peptibody, and the like. Antibody derivatives need not be derived from a specific wild type antibody. For example, one can employ known techniques such as phage display to generate and select for small proteins having a binding domain similar to an antibody complementarity-determining region (CDR). In some embodiments, the antigen-binding moiety includes an scFv. The binding domain can also be derived from a natural or synthetic ligand or receptor, whether soluble or membrane-bound, that specifically binds to the target surface protein, for example without limitation, PD-1, EGF, and the like.

The antigen-binding moiety can include naturally-occurring amino acid sequences or can be engineered, designed, or modified so as to provide desired and/or improved properties, e.g., binding affinity. Generally, the binding affinity of an antigen-binding moiety, e.g., an antibody, for a target antigen (e.g., CD19 antigen) can be calculated by the Scatchard method described by Frankel et al., Mol Immunol (1979) 16:101-06. In some embodiments, binding affinity is measured by an antigen/antibody dissociation rate. In some embodiments, binding affinity is measured by a competition radioimmunoassay. In some embodiments, binding affinity is measured by ELISA. In some embodiments, antibody affinity is measured by flow cytometry. In some embodiments, binding affinity is measured by bio-layer interferometry. An antibody that selectively binds an antigen (such as CD19) when it is capable of binding that antigen with high affinity, without significantly binding other antigens.

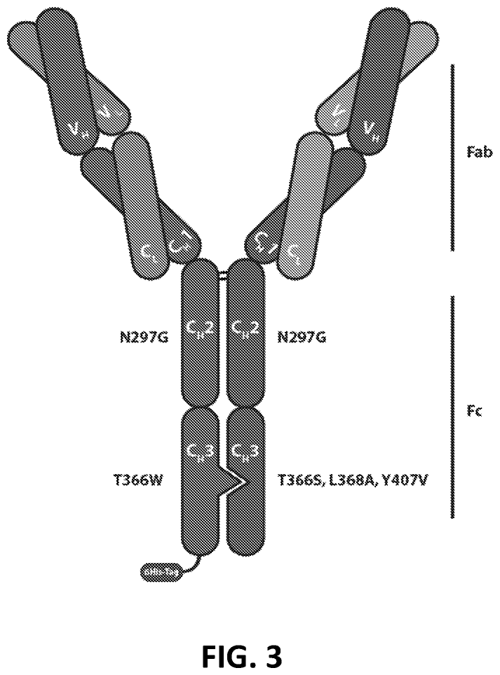

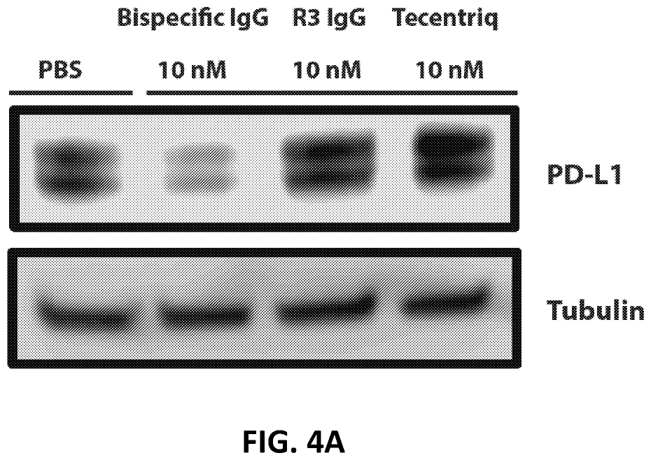

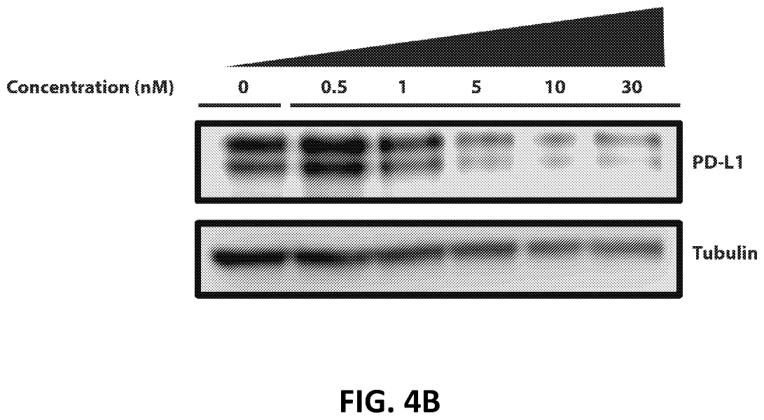

Bispecific antibodies can be prepared by known methods. Embodiments of the disclosure include “knob-into-hole” bispecific antibodies, wherein the otherwise symmetric dimerization region of a bispecific binding agent is altered so that it is asymmetric. For example, a knob-into-hole bispecific IgG that is specific for antigens A and B can be altered so that the Fc portion of the A-binding chain has one or more protrusions (“knobs”), and the Fc portion of the B-binding chain has one or more hollows (“holes”), where the knobs and holes are arranged to interact. This reduces the homodimerization (A-A and B-B antibodies), and promotes the heterodimerization desired for a bispecific binding agent. See, e.g., Y. Xu et al., mAbs (2015) 7(1):231-42. In some embodiments, the bispecific binding agent has a knob-into-hole design. In some embodiments, the “knob” comprises a T336W alteration of the CH3 domain, i.e., the threonine at position 336 is replaced by a tryptophan. In some embodiments, the “hole” comprises one or a combination of T366S, L368A, and Y407V. In some embodiments, the “hole” comprises T366S, L368A, and Y407V. For example, an illustration is provided in FIG. 3. In some embodiments, the “knob” constant region comprises SEQ ID NO: 14. In some embodiments, the heavy chain Fc “knob” constant region has a histidine tag. In some embodiments, the heavy chain Fc “hole” constant region comprises SEQ ID NO: 15. In certain embodiments, an exemplary CH2-CH3 domain sequence of a Knob construct with N297G is provided in SEQ ID NO.: 335. In other embodiments, an exemplary CH2-CH3 domain sequence of a Hole construct with N297G is provided in SEQ ID NO.: 336. In some embodiments, an exemplary wildtype CH2-CH3 domain sequence is provided in SEQ ID NO.: 337. In other embodiments, the “knob” and the “hole” constant regions comprise sequences that are about 70%, 75%, 80%, 85%, 90%, 95%, 99% identical to the sequences provided herein.

In some embodiments, the bispecific binding agent comprises a fusion protein having two binding domains. In some embodiments, the E3 binding domain comprises a half antibody, a Fab, a single chain Fab, or an scFv. In some embodiments, the E3 binding domain comprises a half IgG. In some embodiments, the target surface protein binding domain comprises a half antibody, a Fab, a single chain Fab, or an scFv, independently of the choice of form for the E3 binding domain. In some embodiments, the E3 binding domain comprises a half antibody, and the target surface protein binding domain comprises a half antibody. In some embodiments, the half antibodies are each half IgG antibodies. In some embodiments, the half antibodies are each half knob-into-hole IgG antibodies. In some embodiments, the E3 binding domain comprises a half antibody, and the target surface protein binding domain comprises a scFab. In some embodiments, the E3 binding domain comprises a half antibody, and the target surface protein binding domain comprises an scFv. In some embodiments, the E3 binding domain comprises an scFv, and the target surface protein binding domain comprises a scFab.

In some embodiments, the bispecific binding agent comprises an FcRn receptor recognition domain, to promote return of the bispecific binding agent to the extracellular space if the bispecific binding agent is internalized.

### Target Surface Proteins

The bispecific binding agents disclosed herein has a binding affinity for one or more target surface proteins, as well as a membrane-associated E3 ligase. Target surface proteins are selected based on their involvement in immune suppression or the escape of neoplastic cells from immunosurveilance, or their participation in neoplastic cell proliferation or metastasis. Surface proteins that can be targeted according to the methods of the disclosure include proteins such as membrane steroid receptors, EGF receptors, TGF receptors, transferrin receptors, CD19, CD20, CDCP1, and the like. Other suitable target surface proteins include proteins such as PD-L1, PD-L2, CTLA-4, A2AR, B7-H3, B7-H4, BTLA, KIR, LAG3, NKG2D, TIM-3, VISTA, and SIGLEC7, that inhibit attack by immune cells, such as T cells, natural killer cells, macrophages, and the like. In some embodiments, the target surface protein is a protein that is overexpressed by target cells. In some embodiments, the target surface protein is a protein that contributes the the target cell's ability to proliferate, metastasize, or evade the immune system. In some embodiments, the target surface protein is an immune checkpoint protein. In some embodiments, the target surface protein is PD-L1, PD-L2, CTLA-4, A2AR, B7-H3, B7-H4, BTLA, KIR, LAG3, NKG2D, TIM-3, VISTA, or SIGLEC7. In some embodiments, the target surface protein is selected from membrane steroid receptors, EGF receptors, TGF receptors, transferrin receptors, CDCP1, CD19, and CD20.

In some embodiments, the target surface protein is a T cell receptor (TCR) polypeptide, a TCR co-stimulatory surface protein, CD4, CD8, or a CAR-T. Bispecific binding agents with this specificity are useful for down-regulating or suppressing T cells and CAR-T cells.

In some embodiments, the bispecific binding agent is capable of binding a tumor-associated antigen (TAA) or a tumor-specific antigen (TSA). TAAs include a molecule, such as, for example, a protein present on tumor cells and on a sub-population of normal cells, or on many normal cells, but at much lower concentration than on tumor cells. Examples include, without limitation, CEA, AFP, HER2, CTAG1B and MAGEA1. In contrast, TSAs generally include a molecule, such as a protein present on tumor cells but not expressed on normal cells. Examples include, without limitation, oncoviral antigens and mutated proteins (also known as neoantigens).

In some cases, the target surface protein binding domain is specific for an epitope present in an antigen that is expressed by a malignant neoplastic cell, e.g., a tumor-associated antigen or a tumor-specific antigen. The tumor-associated or tumor-specific antigen can be an antigen associated with, for example, a breast cancer cell, a B cell lymphoma, a pancreatic cancer, a Hodgkin's lymphoma cell, an ovarian cancer cell, a prostate cancer cell, a mesothelioma, a lung cancer cell, a non-Hodgkin's B-cell lymphoma (B-NHL) cell, an ovarian cancer cell, a prostate cancer cell, a mesothelioma cell, a melanoma cell, a chronic lymphocytic leukemia cell, an acute lymphocytic leukemia cell, a neuroblastoma cell, a glioma, a glioblastoma, a bladder cancer cell, a colorectal cancer cell, and the like. It will also be understood that a tumor-associated antigen may also be expressed by a non-cancerous cell. In some embodiments, the antigen-binding domain is specific for an epitope present in a tissue-specific antigen. In some embodiments, the antigen-binding domain is specific for an epitope present in a disease-associated antigen.

### E3 Ligases

The bispecific binding agent of the disclosure also binds a membrane-associated E3 ligase. E3 ligases useful in the disclosure include those ligases that are found in association with the target cell plasma membrane (cell membrane). These membrane-associated E3 ligases include, for example, RNF43, ZNRF3, RNF128 (GRAIL), MARCH11, and the like. RNF128 is characteristically expressed in T cells; thus the activity of a bispecific binding agent that binds to RNF128 can be limited to T cells and any other cells that express RNF128.

### Exemplary Constructs

In some embodiments, the bispecific binding agent of the present disclosure comprises a binding arm to an E3 ligase and a binding arm to for a target surface protein as provided herein. In some embodiments, the binding arm to an E3 ligase binds to an extracellular protein attached to an E3 ligase or a transmembrane protein that interacts with an E3 ligase.

In certain embodiments, the binding arm to an E3 ligase comprises a light chain and a heavy chain. In some embodiments, the light chain and the heavy chain each comprises a variable domain. In general, the variable regions of the heavy and light chain each consist of four framework regions (FR) connected by complementarity determining regions (CDRs) also known as hypervariable regions. The CDRs in each chain are held together in close proximity by the FRs and, with the CDRs from the other chain, contribute to the formation of the antigen-binding site of binding agents. There are at least two techniques for determining CDRs: (1) an approach based on cross-species sequence variability; and (2) an approach based on crystallographic studies of antigen-antibody complexes. In addition, combinations of these two approaches are sometimes used in the art to determine CDRs.

In some embodiments, the first binding domain comprises heavy chain framework region sequence set forth in SEQ ID NOs.: 12 or 320 and light chain framework region sequence set forth in SEQ ID NOs.: 11 or 319. In some embodiments, the second binding domain comprises heavy chain framework region sequence set forth in SEQ ID NOs.: 12 or 320 and light chain framework region sequence set forth in SEQ ID NOs.: 11 or 319. In some embodiments, the heavy chain and light chain framework region sequence comprise sequences that are about 70%, 75%, 80%, 85%, 90%, 95%, 99% identical to the sequences provided herein.

In some embodiments, the first binding domain comprises light chain variable domain CDR3 (LC-CDR3) sequence and heavy chain variable domain CDR1 (HC-CDR1), HC-CDR2, and HC-CDR3 sequences comprising the sequences set forth in Table 2, respectively, or a variant thereof comprising 1, 2, 3, or 4 conservative amino acid substitutions. In some embodiments, the second binding domain comprises LC-CDR3 sequence and HC-CDR1, HC-CDR2, and HC-CDR3 sequences comprising the sequences set forth in Table 3, respectively, or a variant thereof comprising 1, 2, 3, or 4 conservative amino acid substitutions.

In other embodiments, the first binding domain of the bispecific binding agent comprises a heavy chain variable domain; and the second binding domain of the bispecific binding agent comprises a heavy chain FR sequence set forth in SEQ ID NOs.: 12 or 320 and light chain FR sequence set forth in SEQ ID NOs.: 11 or 319. As used herein, such a bispecific binding agent is also called a “VH binder.” In an exemplary embodiment, the heavy chain variable domain of the first binding domain comprises the FR sequence set forth in SEQ ID NO.: 321. In some embodiments of the VH binder, the first binding domain comprises VH-CDR1, VH-CDR2, and VH-CDR3 sequences set forth in Table 4, respectively, or a variant thereof comprising 1, 2, 3, or 4 conservative amino acid substitutions. In some embodiments of the VH binder, the second binding domain comprises LC-CDR3 sequence and HC-CDR1, HC-CDR2, and HC-CDR3 sequences comprising the sequences set forth in Table 3, respectively, or a variant thereof comprising 1, 2, 3, or 4 conservative amino acid substitutions.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the sequences of the binding agents of the present disclosure do not abrogate the binding of the binding agent containing the amino acid sequence, to the antigen(s), i.e., the E3 ligase and/or the target surface protein to which the binding agent binds. Methods of identifying nucleotide and amino acid conservative substitutions which do not eliminate antigen binding are well-known in the art.

### Synthesis

Bispecific binding agents are synthesized using the techniques of recombinant DNA and protein expression. For example, for the synthesis of DNA encoding a bispecific IgG of the disclosure, suitable DNA sequences encoding the constant domains of the heavy and light chains are widely available. Sequences encoding the selected variable domains are inserted by standard methods, and the resulting nucleic acids encoding full-length heavy and light chains are transduced into suitable host cells and expressed. Alternatively, the nucleic acids can be expressed in a cell-free expression system, which can provide more control over oxidation and reduction conditions, pH, folding, glycosylation, and the like.

Bispecific IgG proteins have two different complementary determining regions (CDRs), each specific for either the target surface protein or the membrane-associated E3 ligase. Thus, two different heavy chains and two different light chains are required. These may be expressed in the same host cell, and the resulting product will contain a mixture of homodimers and bispecific heterodimers. Homodimers can be separated from the bispecific antibodies by affinity purification (for example, first using beads coated with one antigen, then beads coated with the other antigen), reduced to monomers, and reassociated. Alternatively, one can employ a a “knobs into holes” design, in which a dimerization region of a heavy chain constant region is altered so that the surface either protrudes (“knob”) from the surface (as compared to the wild type structure) or forms a cavity (“hole”) in such a way that the two modified surfaces are still capable of dimerizing. The knob heavy chain and its associated light chain are then expressed in one host cell, and the hole heavy chain and associated light chain are expressed in a different host cell, and the expressed proteins are combined. The asymmetry in the dimerization regions promotes the formation of heterodimers. (See, e.g., Example 1 below, and FIG. 3, indicating the alterations made compared to wild type.) To obtain dimerization, the two “monomers” (each consisting of a heavy chain and a light chain) are combined under reducing conditions at a moderately basic pH (e.g., about pH 8 to about pH 9) to promote disulfide bond formation between the appropriate heavy chain domains. See, e.g., U.S. Pat. No. 8,216,805 and EP 1870459A1, incorporated herein by reference.

Other methods can be used to promote heavy-chain heterodimerization of the first and second polypeptide chains of bispecific antibodies. For example, in some embodiments, the heavy-chain heterodimerization of the first and second polypeptide chains of the engineered antibodies as disclosed herein can be achieved by a controlled Fab arm exchange method as described by F. L. Aran et al., Proc Natl Acad Sci USA (2013) 110(13):5145-50.

The dimerization process can result in exchange of the light chains between different heavy chain monomers. One method for avoiding this outcome is to replace the binding region of the antibody with a “single chain Fab”, e.g., wherein the light chain CDR is fused to the heavy chain CDR by a linking polypeptide. The Fab region of an IgG (or other antibody) may also be replaced with an scFv, nanobody, and the like.

The binding activity of the engineered antibodies of the disclosure can be assayed by any suitable method known in the art. For example, the binding activity of the engineered antibodies of the disclosure can be determined by, e.g., Scatchard analysis (Munsen et al., Analyt Biochem (1980) 107:220-39). Specific binding may be assessed using techniques known in the art including but not limited to competition ELISA, BIACORE® assays and/or KINEXA® assays. An antibody that preferentially or specifically binds (used interchangeably herein) to a target antigen or target epitope is a term well understood in the art, and methods to determine such specific or preferential binding are also known in the art. An antibody is said to exhibit specific or preferential binding if it reacts or associates more frequently, more rapidly, with greater duration and/or with greater affinity with a particular antigen or epitope than it does with alternative antigens or epitopes. An antibody specifically or preferentially binds to a target if it binds with greater affinity, avidity, more readily, and/or with greater duration than it binds to other substances. Also, an antibody specifically or preferentially binds to a target if it binds with greater affinity, avidity, more readily, and/or with greater duration to that target in a sample than it binds to other substances present in the sample. For example, an antibody that specifically or preferentially binds to a HER2 epitope is an antibody that binds this epitope with greater affinity, avidity, more readily, and/or with greater duration than it binds to other HER2 epitopes or non-HER2 epitopes. It is also understood by reading this definition, for example, that an antibody which specifically or preferentially binds to a first target antigen may or may not specifically or preferentially bind to a second target antigen. As such, specific binding and preferential binding do not necessarily require (although it can include) exclusive binding.

### Engineered Transmembrane Proteins

The engineered transmembrane proteins disclosed herein have a binding affinity for one or more target surface proteins, and incorporate a domain having a membrane-associated E3 ligase ubiquitin ligase activity. All of the target surface proteins described herein with regard to bispecific binding agents are also suitable targets for the engineered transmembrane proteins. In some embodiments, the engineered transmembrane protein has a binding affinity for CD19, B7H3 (CD276), BCMA, CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Ra, KIT (CD117), MMP14, MUC1, NCAM, PAP, PDGFR-β, PRSS21, PSCA, PSMA, ROR1, SSEA-4, TAG72, TEM1/CD248, TEM7R, TSHR, VEGFR2, BCMA (CD269), ALPI, citrullinated vimentin, cMet, or Axl. In some embodiments, the engineered transmembrane protein has a binding affinity for PD-L1, PD-L2, CTLA-4, A2AR, B7-H3, B7-H4, BTLA, KIR, LAG3, NKG2D, TIM-3, VISTA, or SIGLEC7. In some embodiments, the engineered transmembrane protein has a binding affinity for a membrane steroid receptor, an EGF receptor, a TGF receptor, a transferrin receptor, CD19, or CD20. In some embodiments, the engineered transmembrane protein has a binding affinity for a T cell receptor (TCR) polypeptide, a TCR co-stimulatory surface protein, CD4, CD8, or a CAR-T.

The E3 ligase domain can be selected from any of the E3 ligases described above as a target of the bispecific binding agents. Further, the E3 ligase selected need not be native to, or expressed by, the target cell, as long as the E3 ligase is capable of transferring a ubiquitin or conjugated ubiquitin chain from an endogenous E2 ubiquitin-conjugating enzyme. This permits the use of E3 ligases derived from mammalian species that are different from the species of the target cell, for example, enabling the use of a murine E3 ligase in a human subject. This also prevents a malignant neoplastic cell from escaping the effect of treatment by down-regulating expression of a single E3 ligase; as the engineered transmembrane proteins provided herein include the E3 ligase activity, the malignant neoplastic cell would need to down-regulate or suppress expression of all endogenous E2 ubiquitin-conjugating enzyme in order to avoid the engineered transmembrane protein activity. In some embodiments, the E3 ligase domain comprises a membrane-associated E3 ligase, or a functional portion thereof. In some embodiments, the membrane-associated E3 ligase is a human membrane-associated E3 ligase. In some embodiments, the membrane-associated E3 ligase is RNF43, ZNRF3, RNF128 (GRAIL), or MARCH11.

The engineered transmembrane proteins include a binding domain specific for a selected target surface protein. This binding domain can take the form of any of the target surface protein binding domains described herein, including for example, an antibody, a nanobody, a minibody, a Fab fragment, a single chain variable fragment (scFv), and a single domain antibody (sdAb), or a functional fragment thereof. The binding domain can also be derived from a natural or synthetic ligand or receptor, whether soluble or membrane-bound, that specifically binds to the target surface protein, for example without limitation, PD-1, HER2, HER3, and the like.

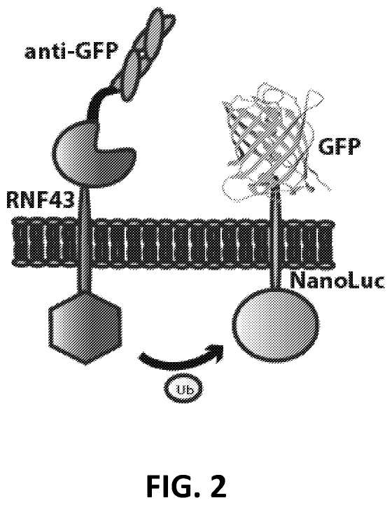

The binding domain and the E3 ligase domain can be expressed together, as a fusion protein, or otherwise associated by a covalent bond (for example, via a disulfide bond between two cysteine residues), or associated by a non-covalent affinity. In some embodiments, the engineered transmembrane protein is a fusion protein. In some embodiments, the E3 ligase domain and the target surface protein binding domain of the engineered transmembrane protein are linked by a disulfide bond. An illustration of an exemplary engineered transmembrane protein having a GFP binding domain and a membrane-associated E3 ligase domain is shown in FIG. 2.

In one exemplary embodiment, the engineered transmembrane protein of the present disclosure comprises an anti-GFP scFab sequence having the sequence of SEQ ID NO: 2 (light chain) and SEQ ID NO: 4 (heavy chain), with the linking domain set provided in SEQ ID NO: 3. A short linker (SEQ ID NO: 5) connects the anti-GFP scFab domain to the RNF43 domain (SEQ ID NO: 6). In this exemplary embodiment, the full sequence of the engineered transmembrane protein is set forth in SEQ ID NO: 1.

In another exemplary embodiment, the reporter construct is assembled from a GFP domain (SEQ ID NO: 8), a transmembrane/linker domain (SEQ ID NO: 9), and a nanoluciferase domain (SEQ ID NO: 10). In this exemplary embodiment, the full sequence of the reporter construct is set forth in SEQ ID NO: 7.

### Immunoconjugates

The present disclosure further comprises immunoconjugates comprising any of the binding agents disclosed herein. In some embodiments, the immunoconjugates of the present disclosure comprise the bispecific binding agents provided herein. In other embodiments, the immunoconjugates of the present disclosure comprise the engineered transmembrane proteins disclosed herein. The term “immunoconjugate” or “conjugate” as used herein refers to a compound or a derivative thereof that is linked to a binding agent, such as the bispecific binding agents or the engineered transmembrane proteins provided herein. The immunoconjugate of the present disclosure generally comprises a binding agent, such as the bispecific binding agents or the engineered transmembrane proteins provided herein and a small molecule. In some embodiments, the immunoconjugate further comprises a linker.

A “linker” is any chemical moiety that is capable of linking a compound, for example, the small molecule disclosed herein, to a binding agent, such as the bispecific binding agents or the engineered transmembrane proteins provided herein in a stable and covalent manner. Linkers can be susceptible to or be substantially resistant to acid-induced cleavage, light-induced cleavage, peptidase-induced cleavage, esterase-induced cleavage, and disulfide bond cleavage, at conditions under which the compound or the antibody remains active. Suitable linkers are well known in the art and include, for example, disulfide groups, thioether groups, acid labile groups, photolabile groups, peptidase labile groups and esterase labile groups. Linkers also include charged linkers, and hydrophilic forms thereof as described herein and known in the art. In certain embodiments, the linker is selected from the group consisting of a cleavable linker, a non-cleavable linker, a hydrophilic linker, and a dicarboxylic acid based linker. In an exemplary embodiment, the linker is a non-cleavable linker. In another exemplary embodiment, the linker is a spacer, such as PEG4. In other embodiments, the small molecule does not dissociate from the binding agent.

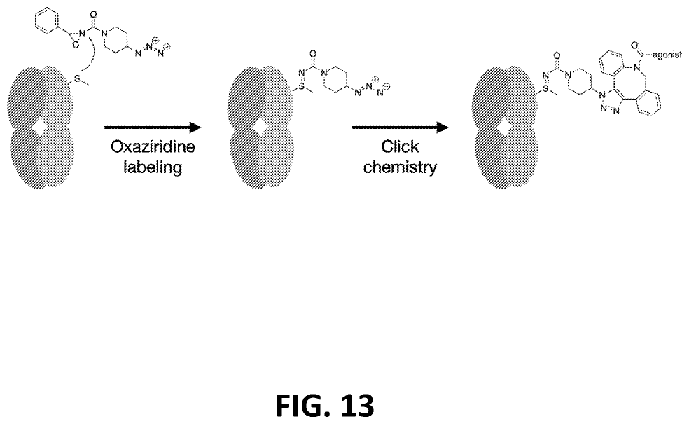

The small molecule encompassed by the present disclosure can be any small molecule one skilled in the art deems suitable for the use, for example, target degradation of a protein of interest. In other exemplary embodiments, the small molecules comprise agonists, such as, without being limited to, CGS21680. In additional exemplary embodiments, the small molecules comprise antagonists, including without being limited to, ZN241385, plerixafor, maraviroc, or aplaviroc. The small molecules can be conjugated to the binding agent, such as the bispecific binding agents or the engineered transmembrane proteins provided herein by methods known in the art. Some exemplary conjugation methods include, without limitations, methionine using oxaziridine based reagents (illustrated in FIG. 13), cysteine labeling with a maleimide based reagent or disulfide exchange reagent, lysine reactive activated esters, utilizing incorporation of an unnatural amino acid containing a reactive handle for conjugation, and N-Terminal or C-terminal conjugation. Some methods use engineered amino acids, such as aldehydes, for reactive conjugation. Other methods include Tag based bioconjugation methods. The present disclosure provides some exemplary methods for conjugation. For instance, see Example 6. It is understood that the present disclosure is not limited by the few examples listed here, and other commonly known conjugation methods can also be used in making the immunoconjugates disclosed herein.

### Nucleic Acid Molecules

In one aspect, some embodiments disclosed herein relate to nucleic acid molecules comprising nucleotide sequences encoding the bispecific binding agents and engineered transmembrane proteins of the disclosure, including expression cassettes, and expression vectors containing these nucleic acid molecules operably linked to heterologous nucleic acid sequences such as, for example, regulatory sequences which direct in vivo expression of the engineered transmembrane protein in a host cell.

Nucleic acid molecules of the present disclosure can be nucleic acid molecules of any length, including nucleic acid molecules that are generally between about 5 Kb and about 50 Kb, for example between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

In some embodiments, the nucleotide sequence is incorporated into an expression cassette or an expression vector. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell or tissue and/or into an individual. Thus, in some embodiments, an expression cassette of the disclosure comprises a nucleotide sequence encoding a bispecific binding agent or an engineered transmembrane protein operably linked to expression control elements sufficient to guide expression of the cassette in vivo. In some embodiments, the expression control element comprises a promoter and/or an enhancer and optionally, any or a combination of other nucleic acid sequences capable of effecting transcription and/or translation of the coding sequence.

In some embodiments, the nucleotide sequence is incorporated into an expression vector. Vectors generally comprise a recombinant polynucleotide construct designed for transfer between host cells, that may be used for the purpose of transformation, i.e., the introduction of heterologous DNA into a host cell. As such, in some embodiments, the vector can be a replicon, such as a plasmid, phage, or cosmid, into which another DNA segment may be inserted so as to bring about the replication of the inserted segment. Expression vectors further include a promoter operably linked to the recombinant polynucleotide, such that the recombinant polynucleotide is expressed in appropriate cells, under appropriate conditions. In some embodiments, the expression vector is an integrating vector, which can integrate into host nucleic acids.

In some embodiments, the expression vector is a viral vector, which further includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. Retroviral vectors contain structural and functional genetic elements, or portions thereof, that are primarily derived from a retrovirus. Lentiviral vectors are viral vectors or plasmids containing structural and functional genetic elements, or portions thereof, including LTRs that are primarily derived from a lentivirus.

The nucleic acid sequences can be optimized for expression in the host cell of interest. For example, the G-C content of the sequence can be adjusted to levels average for a given cellular host, as calculated by reference to known genes expressed in the host cell. Methods for codon optimization are known in the art. Codon usages within the coding sequence of the proteins disclosed herein can be optimized to enhance expression in the host cell, such that about 1%, about 5%, about 10%, about 25%, about 50%, about 75%, or up to 100% of the codons within the coding sequence have been optimized for expression in a particular host cell.

Some embodiments disclosed herein relate to vectors or expression cassettes including a recombinant nucleic acid molecule encoding the proteins disclosed herein. The expression cassette generally contains coding sequences and sufficient regulatory information to direct proper transcription and/or translation of the coding sequences in a recipient cell, in vivo and/or ex vivo. The expression cassette may be inserted into a vector for targeting to a desired host cell and/or into an individual. An expression cassette can be inserted into a plasmid, cosmid, virus, autonomously replicating polynucleotide molecule, or bacteriophage, as a linear or circular, single-stranded or double-stranded, DNA or RNA polynucleotide, derived from any source, capable of genomic integration or autonomous replication, including a nucleic acid molecule where one or more nucleic acid sequences has been linked in a functionally operative manner, i.e., operably linked.

Also provided herein are vectors, plasmids, or viruses containing one or more of the nucleic acid molecules encoding any bispecific binding agent or engineered protein disclosed herein. The nucleic acid molecules can be contained within a vector that is capable of directing their expression in, for example, a cell that has been transformed/transduced with the vector.

Suitable vectors for use in eukaryotic and prokaryotic cells are known in the art and are commercially available, or readily prepared by a skilled artisan. See for example, Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

DNA vectors can be introduced into eukaryotic cells via conventional transformation or transfection techniques. Suitable methods for transforming or transfecting host cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

Viral vectors that can be used in the disclosure include, for example, retrovirus vectors, adenovirus vectors, and adeno-associated virus vectors, lentivirus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

The precise components of the expression system are not critical. For example, a bispecific binding agent as disclosed herein can be produced in a eukaryotic host, such as a mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult P. Jones, “Vectors: Cloning Applications”, John Wiley and Sons, New York, N.Y., 2009).

The nucleic acid molecules provided can contain naturally occurring sequences, or sequences that differ from those that occur naturally but encode the same gene product because the genetic code is degenerate. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., comprising either a sense or an antisense strand).

The nucleic acid molecules are not limited to sequences that encode polypeptides (e.g., antibodies); some or all of the non-coding sequences that lie upstream or downstream from a coding sequence (e.g., the coding sequence of a bispecific binding agent, or engineered transmembrane protein) can also be included. Those of ordinary skill in the art of molecular biology are familiar with routine procedures for isolating nucleic acid molecules. They can, for example, be generated by treatment of genomic DNA with restriction endonucleases, or by the polymerase chain reaction (PCR). In the event the nucleic acid molecule is a ribonucleic acid (RNA), transcripts can be produced, for example, by in vitro transcription.

Recombinant Cells and Cell Cultures

The nucleic acid of the present disclosure can be introduced into a host cell, such as a human B lymphocyte, to produce a recombinant cell containing the nucleic acid molecule. Accordingly, some embodiments of the disclosure relate to methods for making recombinant cells, including the steps of: (a) providing a cell capable of protein expression and (b) contacting the provided cell with any of the recombinant nucleic acids described herein.

Introduction of the nucleic acid molecules of the disclosure into cells can be achieved by viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules are delivered to cells by viral or non-viral delivery vehicles known in the art. For example, the nucleic acid molecule can be stably integrated in the host genome, or can be episomally replicating, or present in the recombinant host cell as a mini-circle expression vector for a stable or transient expression. Accordingly, in some embodiments disclosed herein, the nucleic acid molecule is maintained and replicated in the recombinant host cell as an episomal unit. In some embodiments, the nucleic acid molecule is stably integrated into the genome of the recombinant cell. Stable integration can be completed using classical random genomic recombination techniques or with more precise genome editing techniques such as using guide RNA directed CRISPR/Cas9, or DNA-guided endonuclease genome editing NgAgo (Natronobacterium gregoryi Argonaute), or TALENs genome editing (transcription activator-like effector nucleases). In some embodiments, the nucleic acid molecule present in the recombinant host cell as a mini-circle expression vector for a stable or transient expression.

The nucleic acid molecules can be encapsulated in a viral capsid or a lipid nanoparticle. For example, introduction of nucleic acids into cells may be achieved by viral transduction. In a non-limiting example, adeno-associated virus (AAV) is a non-enveloped virus that can be engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses. An embodiment is an AAV vector encoding the engineered transmembrane protein of the disclosure.

Lentiviral systems are also suitable for nucleic acid delivery and gene therapy via viral transduction. Lentiviral vectors offer several attractive properties as gene-delivery vehicles, including: (i) sustained gene delivery through stable vector integration into host genome; (ii) the ability to infect both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) potentially safer integration site profile; and (vii) a relatively easy system for vector manipulation and production.

In some embodiments, host cells are genetically engineered (e.g., transduced, transformed, or transfected) with, for example, a vector comprising a nucleic acid sequence encoding an engineered transmembrane protein as described herein, either a virus-derived expression vector or a vector for homologous recombination further comprising nucleic acid sequences homologous to a portion of the genome of the host cell. Host cells can be either untransformed cells or cells that have already been transfected with one or more nucleic acid molecules.

In some embodiments, the recombinant cell is a prokaryotic cell or a eukaryotic cell. In some embodiments, the cell is transformed in vivo. In some embodiments, the cell is transformed ex vivo. In some embodiments, the cell is transformed in vitro. In some embodiments, the recombinant cell is a eukaryotic cell. In some embodiments, the recombinant cell is an animal cell. In some embodiments, the animal cell is a mammalian cell. In some embodiments, the animal cell is a human cell. In some embodiments, the cell is a non-human primate cell. In some embodiments, the mammalian cell is an immune cell, a neuron, an epithelial cell, and endothelial cell, or a stem cell. In some embodiments, the recombinant cell is an immune system cell, e.g., a lymphocyte (e.g., a T cell or NK cell), or a dendritic cell. In some embodiments, the immune cell is a B cell, a monocyte, a natural killer (NK) cell, a basophil, an eosinophil, a neutrophil, a dendritic cell, a macrophage, a regulatory T cell, a helper T cell, a cytotoxic T cell, or other T cell. In some embodiments, the immune system cell is a T lymphocyte.

In some embodiments, the cell is a stem cell. In some embodiments, the cell is a hematopoietic stem cell. In some embodiments of the cell, the cell is a lymphocyte. In some embodiments, the cell is a precursor T cell or a T regulatory (Treg) cell. In some embodiments, the cell is a CD34+, CD8+, or a CD4+ cell. In some embodiments, the cell is a CD8+T cytotoxic lymphocyte cell selected from the group consisting of naïve CD8+ T cells, central memory CD8+ T cells, effector memory CD8+ T cells, and bulk CD8+ T cells. In some embodiments of the cell, the cell is a CD4+T helper lymphocyte cell selected from the group consisting of naïve CD4+ T cells, central memory CD4+ T cells, effector memory CD4+ T cells, and bulk CD4+ T cells. In some embodiments, the cell can be obtained by leukapheresis performed on a sample obtained from a human subject.

In another aspect, provided herein are various cell cultures including at least one recombinant cell as disclosed herein, and a culture medium. Generally, the culture medium can be any one of suitable culture media for the cell cultures described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### Pharmaceutical Compositions

In some embodiments, the bispecific binding agents, engineered transmembrane proteins, nucleic acids, and recombinant cells of the disclosure can be incorporated into compositions, including pharmaceutical compositions. Such compositions typically include the bispecific binding agents, engineered transmembrane proteins, nucleic acids, and/or recombinant cells, and a pharmaceutically acceptable excipient, e.g., a carrier.

Bispecific binding agents of the disclosure can be administered using formulations used for administering antibodies and antibody-based therapeutics, or formulations based thereon. Nucleic acids of the disclosure are administered using formulations used for administering oligonucleotides, antisense RNA agents, and/or gene therapies such as CRISPR/Cas9 based therapeutics. Engineered transmembrane proteins are administered as nucleic acids for expression in the target cell, or as a protein in a carrier capable of fusing with the target cell membrane, for example a fusogenic carrier as described below.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to the extent that it can be administered by syringe. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In some embodiments, the bispecific binding agents or the engineered transmembrane proteins of the disclosure are administered by transfection or infection with nucleic acids encoding them, using methods known in the art, including but not limited to the methods described in McCaffrey et al., Nature (2002) 418:6893, Xia et al., Nature Biotechnol (2002) 20:1006-10, and Putnam, Am J Health Syst Pharm (1996) 53:151-60, erratum at Am J Health Syst Pharm (1996) 53:325.

Engineered transmembrane proteins of the disclosure can be administered using a formulation comprising a fusogenic carrier. These are carriers capable of fusing with the plasma membrane of a mammalian cell. Fusogenic carriers include, without limitation, membrane-encapsulated viral particles and carriers based thereon, exosomes and microvesicles (see, e.g., Y. Yang et al., J Extracellular Vessicles (2018) 7:144131), fusogenic liposomes (see, e.g., Bailey et al., U.S. Pat. No. 5,552,155; Martin et al., U.S. Pat. No. 5,891,468; Holland et al., U.S. Pat. No. 5,885,613; and Leamon, U.S. Pat. No. 6,379,698). An embodiment is the formulation comprising an engineered transmembrane protein and a fusogenic carrier.

### Methods of the Disclosure

### Administration of Bispecific Binding Agents

Administration of any one or more of the therapeutic compositions described herein, e.g., bispecific binding agents, engineered transmembrane proteins, nucleic acids, recombinant cells, and pharmaceutical compositions, can be used to treat individuals having a neoplastic disease, such as cancers. In some embodiments, the bispecific binding agents, engineered transmembrane proteins, nucleic acids, recombinant cells, and pharmaceutical compositions are incorporated into therapeutic compositions for use in methods down-regulating or inactivating T cells, such as CAR-T cells.

Accordingly, in one aspect, provided herein are methods for inhibiting an activity of a target cell in an individual, the methods comprising the step of administering to the individual a first therapy including one or more of the bispecific binding agents, engineered transmembrane proteins, nucleic acids, recombinant cells, and pharmaceutical compositions provided herein, wherein the first therapy inhibits an activity of the target cell by degrading a target surface protein. For example, an activity of the target cell may be inhibited if its proliferation is reduced, if its pathologic or pathogenic behavior is reduced, if it is destroyed or killed, or the like. Inhibition includes a reduction of the measured quantity of at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods include administering to the individual an effective number of the recombinant cell as disclosed herein, wherein the recombinant cell inhibits the target cell in the individual by expression of bispecific binding agents. Generally, the target cell of the disclosed methods can be any cell such as, for example an acute myeloma leukemia cell, an anaplastic lymphoma cell, an astrocytoma cell, a B-cell cancer cell, a breast cancer cell, a colon cancer cell, an ependymoma cell, an esophageal cancer cell, a glioblastoma cell, a bladder cancer cell, a glioma cell, a leiomyosarcoma cell, a liposarcoma cell, a liver cancer cell, a lung cancer cell, a mantle cell lymphoma cell, a melanoma cell, a neuroblastoma cell, a non-small cell lung cancer cell, an oligodendroglioma cell, an ovarian cancer cell, a pancreatic cancer cell, a peripheral T-cell lymphoma cell, a renal cancer cell, a sarcoma cell, a stomach cancer cell, a carcinoma cell, a mesothelioma cell, or a sarcoma cell. In some embodiments, the target cell is a pathogenic cell.

Bispecific binding agents of the disclosure are typically administered in solution or suspension formulation by injection or infusion. In an embodiment, a bispecific binding agent is administered by injection directly into a tumor mass. In another embodiment, a bispecific binding agent is administered by systemic infusion.

Some bispecific binding agents of the disclosure are effective at a concentration of 10 nM. Other bispecific binding agents may be most effective at a higher or lower concentration, depending on the binding affinity for each of the ligands, and the degree of expression of each of the ligands. The range of effective concentrations, however, can be determined by one of ordinary skill in the art, using the disclosure and the experimental protocols provided herein. Similarly, using the effective concentration one can determine the effective dose or range of dosages required for administration.

Depending on the disease or disorder to be treated, the severity and extent of the disease, the subject's health, and the co-administration of other therapies, repeated doses may be administered. Alternatively, a continuous administration may be required. It is expected, however, that the bispecific binding agent will remain in proximity to the cell so that each molecule of bispecific binding agent can ubiquitinate and degrade multiple molecules of target surface protein. Thus, the bispecific binding agents of the disclosure may require lower doses, or less frequent administration, than therapies based on antibody competitive binding.

### Administration of Recombinant Cells to an Individual

In some embodiments, the methods involve administering the recombinant cells to an individual who is in need of such method. This administering step can be accomplished using any method of implantation known in the art. For example, the recombinant cells can be injected directly into the individual's bloodstream by intravenous infusion or otherwise administered to the individual.

The terms “administering”, “introducing”, and “transplanting” are used interchangeably herein to refer to methods of delivering recombinant cells expressing the bispecific binding agents provided herein to an individual. In some embodiments, the methods comprise administering recombinant cells to an individual by a method or route of administration that results in at least partial localization of the introduced cells at a desired site such that a desired effect(s) is/are produced. The recombinant cells or their differentiated progeny can be administered by any appropriate route that results in delivery to a desired location in the individual where at least a portion of the administered cells or components of the cells remain viable. The period of viability of the cells after administration to an individual can be as short as a few hours, e.g., twenty-four hours, to a few days, to as long as several years, or even long-term engraftment for the life time of the individual.

When provided prophylactically, in some embodiments, the recombinant cells described herein are administered to an individual in advance of any symptom of a disease or condition to be treated. Accordingly, in some embodiments the prophylactic administration of a recombinant stem cell population serves to prevent the occurrence of symptoms of the disease or condition.

When provided therapeutically in some embodiments, recombinant stem cells are provided at (or after) the onset of a symptom or indication of a disease or condition, e.g., upon the onset of disease or condition.

For use in the various embodiments described herein, an effective amount of recombinant cells as disclosed herein, can be at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The recombinant cells can be derived from one or more donors or can be obtained from an autologous source (i.e., the human subject being treated). In some embodiments, the recombinant cells are expanded in culture prior to administration to an individual in need thereof.

In some embodiments, the delivery of a composition comprising recombinant cells (i.e., a composition comprising a plurality of recombinant cells a bispecific binding agent provided herein) into an individual by a method or route results in at least partial localization of the cell composition at a desired site. A cell composition can be administered by any appropriate route that results in effective treatment in the individual, e.g., administration results in delivery to a desired location in the individual where at least a portion of the composition delivered, e.g., at least 1×10⁴ cells, is delivered to the desired site for a period of time. Modes of administration include injection, infusion, instillation, and the like. Injection modes include, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, administration by injection or infusion can be made.

In some embodiments, the recombinant cells are administered systemically, in other words a population of recombinant cells are administered other than directly into a target site, tissue, or organ, such that it enters, instead, the individual's circulatory system and, thus, is subject to metabolism and other like processes.

The efficacy of a treatment with a composition for the treatment of a disease or condition can be determined by the skilled clinician. However, one skilled in the art will appreciate that a treatment is considered effective treatment if any one or all of the signs or symptoms or markers of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting disease progression, e.g., arresting, or slowing the progression of symptoms; or (2) relieving the disease, e.g., causing regression of symptoms; and (3) preventing or reducing the likelihood of the development of symptoms.

As discussed above, a therapeutically effective amount includes an amount of a therapeutic composition that is sufficient to promote a particular effect when administered to an individual, such as one who has, is suspected of having, or is at risk for a disease. In some embodiments, an effective amount includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

The efficacy of a treatment including a disclosed therapeutic composition for the treatment of disease can be determined by the skilled clinician. However, a treatment is considered effective if at least any one or all of the signs or symptoms of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; (2) relieving the disease, e.g., causing regression of symptoms; or (3) preventing or reducing the likelihood of the development of symptoms.

In some embodiments, the individual is a mammal. In some embodiments, the mammal is human. In some embodiments, the individual has or is suspected of having a disease associated with cell signaling mediated by a cell surface protein. In some embodiments, the disease is a cancer or a chronic infection.

### Systems and Kits

Also provided herein are systems and kits including the bispecific binding agents, engineered transmembrane proteins, recombinant nucleic acids, recombinant cells, or pharmaceutical compositions provided and described herein as well as written instructions for making and using the same. For example, provided herein, in some embodiments, are systems and/or kits that include one or more of: a bispecific binding agent as described herein, an engineered transmembrane protein as described herein, a recombinant nucleic acid as described herein, a recombinant cell as described herein, or a pharmaceutical composition as described herein. In some embodiments, the systems and/or kits of the disclosure further include one or more syringes (including pre-filled syringes) and/or catheters used to administer one any of the provided bispecific binding agents, engineered transmembrane proteins, recombinant nucleic acids, recombinant cells, or pharmaceutical compositions to an individual. In some embodiments, a kit can have one or more additional therapeutic agents that can be administered simultaneously or sequentially with the other kit components for a desired purpose, e.g., for modulating an activity of a cell, inhibiting a target cancer cell, or treating a disease in an individual in need thereof.

Any of the above-described systems and kits can further include one or more additional reagents, where such additional reagents can be selected from: dilution buffers; reconstitution solutions, wash buffers, control reagents, control expression vectors, negative control polypeptides, positive control polypeptides, reagents for in vitro production of the bispecific binding agents or engineered transmembrane protein.

In some embodiments, a system or kit can further include instructions for using the components of the kit to practice the methods. The instructions for practicing the methods are generally recorded on a suitable recording medium. For example, the instructions can be printed on a substrate, such as paper or plastic, and the like. The instructions can be present in the kits as a package insert, in the labeling of the container of the kit or components thereof (i.e., associated with the packaging or sub-packaging), and the like. The instructions can be present as an electronic storage data file present on a suitable computer readable storage medium, e.g. CD-ROM, diskette, flash drive, and the like. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### EXAMPLES

The practice of the present disclosure will employ, unless otherwise indicated, conventional techniques of molecular biology, microbiology, cell biology, biochemistry, nucleic acid chemistry, and immunology, which are well known to those skilled in the art. Such techniques are explained fully in the literature cited above.

Additional embodiments are disclosed in further detail in the following examples, which are provided by way of illustration and are not in any way intended to limit the scope of this disclosure or the claims.

### Example 1

### Synthesis of Bispecific Binding Agents and Engineered Transmembrane Proteins

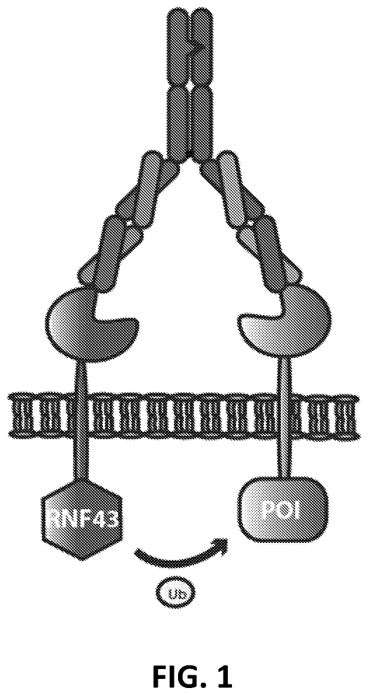

This Example describes experiments performed to generate each of the following types of constructs: bispecific IgG, bispecific IgG with a single chain Fab on one arm, and a Fab-scFV fusion. A graphical representation of the bispecific degrader mode of action can be found in FIG. 1, and a graphical representation of an engineered transmembrane protein having an anti-GFP domain control fused to RNF43 is provided in FIG. 2.

### Half IgG Expression

For half IgG expression of these constructs, the following 6-day process was undertaken. At day one, 7.5×10⁷ Expi293F™ cells (ThermoFisher Scientific) were split in 25.5 mL of Expi293™ Growth Medium, in a 125 mL flask per transfection. The Expi™ transfection reagents were used per the manufacturer's protocol: first, 1.5 mL of OptiMEM™ were added to a 15 mL tube, to which 30 μg plasmid DNA were added and mixed (15 μg of each heavy and light chain plasmids). In separate tubes, 1.5 mL OptiMEM™ were aliquoted for each transfection, and 81 μL ExpiFectamine™ reagent were added in each tube for each transfection. The solution was mixed and incubated for 5 min at room temperature (RT). Then, DNA was added to ExpiFectamine™ (3 mL final volume) and incubated for 20 minutes. Finally, the 3 mL of DNA+ExpiFectamine™ mixture were added in OptiMEM™ to each culture flask, containing the Expi293F™ cells. The final culture volume for each transfection was 28.5 mL (+3 mL of 1 mM biotin for Fc fusion).

At day two, 20 hours after the last step of day one, ExpiFectamine™ enhancers were added to each culture, for a final culture volume of 30 mL. ExpiFectamine™ Transfection Enhancer 1 (150 μL), and ExpiFectamine™ Transfection Enhancer 2 (1.5 mL) were then added to each flask.

At day six, the cultures were spun down at 4137 rpm, 4×g for 20 minutes at 4° C. in a centrifuge. Half IgGs were then purified using a common protein A protocol, and the final knob or hole constructs were recovered by buffer exchange into 10 nM Tris pH 7.5, 100 mM NaCl.

### Half IgG In-Vitro Assembly

For half IgG in vitro assembly of these constructs, and to generate a bispecific IgG, a one to one mixture of half IgG knob constructs and half IgG hole constructs (see Table 2 and Table 5 below) was prepared in 10 nM Tris, 100 mM NaCl, pH 7.5. The pH of the mixture was adjusted to about 8.5 with addition of 20% 800 mM L-Arg pH 10. A 200-fold excess of reduced glutathione in 800 mM L-Arg, pH 10, was added to the mixture, and incubated at 37° C. for 16 hours. After the 16 hour incubation, the bispecific IgG was buffer exchanged into phosphate-buffered saline buffer (PBS) using a 30 kDa spin concentrator. Finally, the bispecific IgG was purified via his-tag purification.

Generation of scFab-Based Bispecific IgG and Bispecific Fab-scFv

For the generation of scFab-based bispecific IgG and bispecific Fab-scFv, the following 6-day process was followed.

At day one, 7.5×10⁷ Expi293F™ cells were split in 25.5 mL of Expi293™ Growth Medium, in a 125 mL flask per transfection. The Expi™ transfection reagents were used as per manufacturer's protocol: first, 1.5 mL of OptiMEM™ were added to a 15 mL tube, to which 30 μg plasmid DNA were added and mixed (15 μg of each heavy and light chain plasmids). In separate tubes, 1.5 mL OptiMEM™ were aliquoted for each transfection, and 81 μL ExpiFectamine™ reagent were added in each tube for each transfection. The solution was mixed and incubated for 5 min at room temperature (RT). Then, DNA was added to ExpiFectamine™ (3 mL final volume) and incubated for 20 minutes. Finally, the 3 mL of DNA+ExpiFectamine™ mixture were added in OptiMEM™ to each culture flask, containing the Expi293F™ cells. The final culture volume for each transfection was 28.5 mL (+3 mL of 1 mM biotin for Fc fusion).

At day two, 20 hours after the last step of day one, ExpiFectamine™ enhancers were added to each culture, for a final culture volume of 30 mL. After this, 150 μL of ExpiFectamine™ Transfection Enhancer 1, and 1.5 mL ExpiFectamine™ Transfection Enhancer 2 were added to each flask.

At day six, the cultures were spun down at 4000 rpm for 20 minutes in a centrifuge. The bispecific Fab-scFv constructs were Protein A-purified, and the scFab-based bispecific IgGs were his tag purified. The final constructs were recovered by buffer exchange into PBS.

### Generated Constructs

The constructs generated in the procedures set forth above include the following:

TABLE 1
Constructs Generated

| Type of construct | Construct name |
|---|---|
| Bispecific IgGs | RNF43-PDL1 |
| | RNF43-EGFR |
| | RNF43-CDCP1 |
| | RNF43-CTLA |
| | RNF43-Her2 |
| | ZNRF3-Her2 |
| | ZNRF3-PDL1 |
| | RNF43-MMP14-binder 1 |
| | RNF43-MMP14-binder 2 |
| Bispecific IgG with a single chain Fab on | RNF43-PDL1 |
| one arm (scFab-based bispecific IgG) | |
| Fab-scFV fusion | RNF43-PDL1 |
| | RNF43-Her2 |
| | ZNRF3-Her2 |

Constructs for the E3 ligase arms from Table 1 were prepared using the light chain framework region of SEQ ID NO: 11, the heavy chain Fab framework region of SEQ ID NO: 12, the bispecific IgG with single chain Fab on one arm of SEQ ID NO: 13, the heavy chain Fc “knob” constant region of SEQ ID NO: 14 having a His tag, and the Fab-scFv construct—heavy chain scFv fusion of SEQ ID NO: 16 and 17. The LC-CDR3, HC-CDR1, HC-CDR2, and HC-CDR3 of A5 in Table 2 were used for all of the RNF43 binding arm (also called the RNF43 binder). The LC-CDR3, HC-CDR1, HC-CDR2, and HC-CDR3 of A22 in Table 2 were used for all of the ZNRF3 binding arm.

Constructs for target proteins were prepared using the light chain constant region of SEQ ID NO: 11, the heavy chain Fab constant region of SEQ ID NO: 12, the bispecific IgG with single chain Fab on one arm of SEQ ID NO: 13, the heavy chain Fc “hole” constant region of SEQ ID NO: 15, and the Fab-scFv construct—heavy chain scFv fusion of SEQ ID NO: 16 and 17. The LC and HC variable domains used for target surface proteins PD-L1, HER2, EGFR, CTLA-4, MMP14, and CDCP1 are shown in Table 5 below.

Additional LC-CDR3, HC-CDR1, HC-CDR2, and HC-CDR3 used for E3 ligases RNF43, ZNRF3, and GRAIL (RNF128) are shown in Table 2 below. Additional constructs were prepared using the light chain constant region of SEQ ID NO: 319 and the heavy chain Fab constant region of SEQ ID NO: 320.

In another example, a Fab binding arm to RNF43 was replaced with a VH binder. The sequences for the VH framework regions are provided in SEQ ID NO: 321. The VH CDR sequences for RNF43 are provided in Table 4.

TABLE 2
E3 ligase CDR Combinations

| | E3 ligase | | | | |
|---|---|---|---|---|---|
| Construct | antigen | LC-CDR3 | HC-CDR1 | HC-CDR2 | HC-CDR3 |
| A1 | RNF43 | YYDSSYALF | ISYYSI | SIYPYYGYTY | GSYFYGM (SEQ ID NO: 152) |
| | | (SEQ ID | (SEQ ID | (SEQ ID | |
| | | NO: 18) | NO: 61) | NO: 120) | |
| A2 | RNF43 | SGWPF (SEQ | IYSYYM | YISPYYSYTY | AYADSWPGYSWGSSDFAL |
| | | ID NO: 19) | (SEQ ID | (SEQ ID | (SEQ ID NO: 153) |
| | | | NO: 62) | NO: 121) | |
| A3 | RNF43 | GYSDLI | IYYYSI | SIYSSSGYTS | YPYWYFDGF (SEQ ID |
| | | (SEQ ID | (SEQ ID | (SEQ ID | NO: 154) |
| | | NO: 20) | NO: 63) | NO: 122) | |
| A4 | RNF43 | VYPPI (SEQ | LSYSYI | SISPSYGYTY | PYHPFGGHYWWPYYYHGL |
| | | ID NO: 21) | (SEQ ID | (SEQ ID | (SEQ ID NO: 155) |
| | | | NO: 64) | NO: 123) | |
| A5 | RNF43 | AYPI (SEQ | IYYYSM | SISPYYSYTS | YGYYGWDYHRYSAF (SEQ |
| | | ID NO: 22) | (SEQ ID | (SEQ ID | ID NO: 156) |
| | | | NO: 65) | NO: 124) | |
| A6 | RNF43 | SKYSNQLI | VSYYYI | SIYSSYGSTY | EYYFGL (SEQ ID NO: 157) |
| | | (SEQ ID | (SEQ ID | (SEQ ID | |
| | | NO: 23) | NO: 66) | NO: 125) | |
| A7 | RNF43 | WSWPYPL | FYSYSI | SISSSSGSTS | WSWYNHGSSSWAM (SEQ |
| | | (SEQ ID | (SEQ ID | (SEQ ID | ID NO: 158) |
| | | NO: 24) | NO: 67) | NO: 126) | |
| A8 | RNF43 | SSFIWPL | FYSYSI | SISPYYGSTS | WSYWYSSYYGAM (SEQ ID |
| | | (SEQ ID | (SEQ ID | (SEQ ID | NO: 159) |
| | | NO: 25) | NO: 68) | NO: 127) | |
| A9 | RNF43 | SHWEKLI | IYSYYI | SIYSYYGSTY | IFAMGL (SEQ ID NO: 160) |
| | | (SEQ ID | (SEQ ID | (SEQ ID | |
| | | NO: 26) | NO: 69) | NO: 128) | |
| A10 | RNF43 | GRSWPV | FYSYSI | SISSYYGSTS | NGYNWGM (SEQ ID NO: 161) |
| | | (SEQ ID | (SEQ ID | (SEQ ID | |
| | | NO: 27) | NO: 70) | NO: 129) | |
| A11 | RNF43 | KVRWPLI | IYSSSI (SEQ | SISSYYGYTS | SYWQSYMAM (SEQ ID |
| | | (SEQ ID | ID NO: 71) | (SEQ ID | NO: 162) |
| | | NO: 28) | | NO: 130) | |
| A12 | RNF43 | SSKGLI | VSSSSI | SISSYSGYTY | DIQMDSGYKWHPWLGM |
| | | (SEQ ID | (SEQ ID | (SEQ ID | (SEQ ID NO: 163) |
| | | NO: 29) | NO: 72) | NO: 131) | |
| A13 | RNF43 | ALYYPI | VYSSSI | YISSYSGSTY | SPYGHWYGYYGRQGGL |
| | | (SEQ ID | (SEQ ID | (SEQ ID | (SEQ ID NO: 164) |
| | | NO: 30) | NO: 73) | NO: 132) | |
| A14 | RNF43 | SYYWPV | VYYSSI | SISSYYSYTS | YYFYHSYGSYAL (SEQ ID |
| | | (SEQ ID | (SEQ ID | (SEQ ID | NO: 165) |
| | | NO: 31) | NO: 74) | NO: 133) | |
| A15 | RNF43 | YVYSYPF | IYYSYI | SISSYSGYTS | EWYVGM (SEQ ID NO: 166) |
| | | (SEQ ID | (SEQ ID | (SEQ ID | |
| | | NO: 32) | NO: 75) | NO: 134) | |
| A16 | RNF43 | WWYFPI | VSYSSI | SISSYYGSTS | SYSYTGM (SEQ ID NO: 167) |
| | | (SEQ ID | (SEQ ID | (SEQ ID | |
| | | NO: 33) | NO: 76) | NO: 129) | |
| A17 | ZNRF3 | SSSPF (SEQ | ISYSSI (SEQ | YISSYYGSTS | GWYPYSYSRDAM (SEQ ID |
| | | ID NO: 34) | ID NO: 77) | (SEQ ID | NO: 168) |
| | | | | NO: 135) | |
| A18 | ZNRF3 | SYYPI (SEQ | LYYSYI | SIYPSYGSTY | GYAI (SEQ ID NO: 169) |
| | | ID NO: 35) | (SEQ ID | (SEQ ID | |
| | | | NO: 78) | NO: 136) | |
| A19 | ZNRF3 | SSYYFWSPI | LYYSSI | SISPSYSYTS | SWVYSWGM (SEQ ID |
| | | (SEQ ID | (SEQ ID | (SEQ ID | NO: 170) |
| | | NO: 36) | NO: 79) | NO: 137) | |
| A20 | ZNRF3 | YAYYSPF | ISYYSM | SISPYYGYTS | WVGYYPPYYFSGSYGM |
| | | (SEQ ID | (SEQ ID | (SEQ ID | (SEQ ID NO: 171) |
| | | NO: 37) | NO: 80) | NO: 138) | |
| A21 | ZNRF3 | SYYSLF | LSSYSI | SISPYYGYTS | RYSYSYWGFHPAF (SEQ ID |
| | | (SEQ ID | (SEQ ID | (SEQ ID | NO: 172) |
| | | NO: 38) | NO: 81) | NO: 138) | |
| A22 | ZNRF3 | GWVVPI | ISYYYM | SIYPYYSSTY | DVDWPYYFYAI (SEQ ID |
| | | (SEQ ID | (SEQ ID | (SEQ ID | NO: 173) |
| | | NO: 39) | NO: 82) | NO: 139) | |
| A23 | ZNRF3 | SWDSLI | ISSSSM | YIYPYYGSTS | GAYGAPFYYYYFWWDRGM |
| | | (SEQ ID | (SEQ ID | (SEQ ID | (SEQ ID NO: 174) |
| | | NO: 40) | NO: 83) | NO: 140) | |
| A24 | ZNRF3 | SRRYPV | ISYSSI (SEQ | SISPYYGYTS | NSSYPYSWGSKYSWLAL |
| | | (SEQ ID | ID NO: 84) | (SEQ ID | (SEQ ID NO: 175) |
| | | NO: 41) | | NO: 138) | |
| A25 | ZNRF3 | YSGSLI | VYSYYI | SIYSYYSSTS | SGWGWLYYWYPHGI (SEQ |
| | | (SEQ ID | (SEQ ID | (SEQ ID | ID NO: 176) |
| | | NO: 42) | NO: 85) | NO: 141) | |
| A26 | ZNRF3 | SPYELI (SEQ | VYSSYI | SISSYYSYTS | SSIYYAM (SEQ ID NO: 177) |
| | | ID NO: 43) | (SEQ ID | (SEQ ID | |
| | | | NO: 86) | NO: 133) | |
| A27 | ZNRF3 | SSSDPI (SEQ | VYSSSI | SISSYYGSTS | SIQLAKWGYYWIGSSGM |
| | | ID NO: 44) | (SEQ ID | (SEQ ID | (SEQ ID NO: 178) |
| | | | NO: 87) | NO: 129) | |
| A28 | ZNRF3 | SRRYPV | VSYSSI | SISPSYGSTY | YKVYHWPVQWQRYWPAM |
| | | (SEQ ID | (SEQ ID | (SEQ ID | (SEQ ID NO: 179) |
| | | NO: 45) | NO: 88) | NO: 142) | |
| A29 | ZNRF3 | GYKGSSLI | VYYSSI | SISSYYSSTS | QSMSYWSRQYGF (SEQ ID |
| | | (SEQ ID | (SEQ ID | (SEQ ID | NO: 180) |
| | | NO: 46) | NO: 89) | NO: 143) | |
| A30 | ZNRF3 | SWGWPI | FSSYSI | SISSYYGYTS | DWYYVSGYYFSAF (SEQ ID |
| | | (SEQ ID | (SEQ ID | (SEQ ID | NO: 181) |
| | | NO: 47) | NO: 90) | NO: 130) | |
| A31 | ZNRF3 | PYPGMQPI | FYYYSI | SISPYYGSTY | QPWMYWWLKYAI (SEQ ID |
| | | (SEQ ID | (SEQ ID | (SEQ ID | NO: 182) |
| | | NO: 48) | NO: 91) | NO: 144) | |
| A32 | ZNRF3 | MSSSPI | ISSSYI (SEQ | SIYSYYGSTS | SWWEYFYPYGWYQYAI |
| | | (SEQ ID | ID NO: 92) | (SEQ ID | (SEQ ID NO: 183) |
| | | NO: 49) | | NO: 145) | |
| A33 | ZNRF3 | SSSSLI (SEQ | FYSSYI | SISPYYGSTS | KPWYSERFYQGIHYTAM |
| | | ID NO: 50) | (SEQ ID | (SEQ ID | (SEQ ID NO: 184) |
| | | | NO: 93) | NO: 127) | |
| A34 | ZNRF3 | SSHYLI | FYSYSI | SIYSYYGYTS | SWYPQYDWRYYAL (SEQ |
| | | (SEQ ID | (SEQ ID | (SEQ ID | ID NO: 185) |
| | | NO: 51) | NO: 94) | NO: 146) | |
| A35 | ZNRF3 | YSFSSL | VSSSSI | SISPYSGYTS | EEWYSSGMWWYSYGGI |
| | | (SEQ ID | (SEQ ID | (SEQ ID | (SEQ ID NO: 186) |
| | | NO: 52)I | NO: 95) | NO: 147) | |
| A36 | ZNRF3 | SRRYPV | ISYSSI (SEQ | SISPYYGYTS | NSSYPYSWGSKYSWLAL |
| | | (SEQ ID | ID NO: 96) | (SEQ ID | (SEQ ID NO: 175) |
| | | NO: 53) | | NO: 138) | |
| A37 | ZNRF3 | TWSVVPI | VYSYYI | SISSYYGYTS | YYWGYKGHYPAI (SEQ ID |
| | | (SEQ ID | (SEQ ID | (SEQ ID | NO: 187) |
| | | NO: 54) | NO: 97) | NO: 130) | |
| A38 | ZNRF3 | SRRYPV | ISYSSI (SEQ | SISPYYGYTS | NSSYPYSWGSKYSWLAL |
| | | (SEQ ID | ID NO: 98) | (SEQ ID | (SEQ ID NO: 175) |
| | | NO: 55) | | NO;138) | |
| A39 | GRAIL | SHPAFPF | NLYSYSIH | SISSSYGYTY | TVRGSKKPYFSGWAM (SEQ |
| | | (SEQ ID | (SEQ ID | (SEQ ID | ID NO: 188) |
| | | NO: 56) | NO: 99) | NO: 148) | |
| A40 | GRAIL | GGGWYPF | NIYYSSMH | SIYPYYGSTY | HHSYFFGGL (SEQ ID |
| | | (SEQ ID | (SEQ ID | (SEQ ID | NO: 189) |
| | | NO: 57) | NO: 100) | NO: 149) | |
| A41 | GRAIL | SHAYPI | NLYSYYMH | SISPYSRYTS | DPYFSHVFSYWGF (SEQ ID |
| | | (SEQ ID | (SEQ ID | (SEQ ID | NO: 190) |
| | | NO: 58) | NO: 101) | NO: 150) | |
| A42 | GRAIL | YYYLI (SEQ | NIYYYSMH | SIYPYYSYTY | DFFSSYYPVVAASAGI (SEQ |
| | | ID NO: 59) | (SEQ ID | (SEQ ID | ID NO: 191) |
| | | | NO: 102) | NO: 151) | |
| A43 | GRAIL | HHSLI (SEQ | NISYSSMH | SIYPYYSSTY | DSPYSYYSPWGGM (SEQ ID |
| | | ID NO: 60) | (SEQ ID | (SEQ ID | NO: 192) |
| | | | NO: 103) | NO: 139) | |

TABLE 3
Exemplary LC-CDR1, HC-CDR1, HC-CDR2, and HC-CDR3 sequences for target
antigen PD-L1.

| | Target | | | | |
|---|---|---|---|---|---|
| Construct | Antigen | LC-CDR3 | HC-CDR1 | HC-CDR2 | HC-CDR3 |
| 1 | PD-L1 | YYHPI (SEQ ID | LSYYSI | SISPYYSYTS | SPWDPWAHHGHGI (SEQ ID |
| | | NO: 193) | (SEQ ID | (SEQ ID | NO: 264) |
| | | | NO: 225) | NO: 124) | |
| 2 | PD-L1 | SYYPF (SEQ ID | LYSYYI | SISPSYGYTS | GVASYYYSASYSWYGGM |
| | | NO: 194) | (SEQ ID | (SEQ ID | (SEQ ID NO: 265) |
| | | | NO: 226) | NO: 243) | |
| 3 | PD-L1 | AYYSPI (SEQ ID | LSYSYM | SISPSSSYTY | YYYWHYFWDAF (SEQ ID |
| | | NO: 195) | (SEQ ID | (SEQ ID | NO: 266) |
| | | | NO: 227) | NO: 244) | |
| 4 | PD-L1 | HYTTPP (SEQ ID | IKDTYI | RIYPTNGYTR | |
| | | NO: 196) | (SEQ ID | (SEQ ID | |
| | | | NO: 228) | NO: 245) | |
| 5 | PD-L1 | AWGPF (SEQ ID | ISYYSI | SISPYYGYTS | SPYAPGYFAVHGAPVGGM |
| | | NO: 197) | (SEQ ID | (SEQ ID | (SEQ ID NO: 267) |
| | | | NO: 61) | NO: 138) | |
| 6 | PD-L1 | SSPYLF (SEQ ID | LSSSYM | SIYSYSGYTS | GSYRFWDAF (SEQ ID |
| | | NO: 198) | (SEQ ID | (SEQ ID | NO: 268) |
| | | | NO: 229) | NO: 246) | |
| 7 | PD-L1 | YRYADALI (SEQ | ISSYYI | SIYPYSSYTS | HSYSSGF (SEQ ID NO: 269) |
| | | ID NO: 199) | (SEQ ID | (SEQ ID | |
| | | | NO: 230) | NO: 247) | |
| 8 | PD-L1 | SRYVSPI (SEQ ID | ISSYSI (SEQ | SISSSSSYTS | GYFWSYSGF (SEQ ID |
| | | NO: 200) | ID NO: 231) | (SEQ ID | NO: 270) |
| | | | | NO: 248) | |
| 9 | PD-L1 | YGSYPI (SEQ ID | LSSSYI | SIYPSSSYTS | SDHGVAYGI (SEQ ID |
| | | NO: 201) | (SEQ ID | (SEQ ID | NO: 271) |
| | | | NO: 232) | NO: 249) | |
| 10 | PD-L1 | DYYPYWHAPF | ISSYSM | SISSSYGYTS | GASSDWYFWSSGL (SEQ ID |
| | | (SEQ ID NO: 202) | (SEQ ID | (SEQ ID | NO: 272) |
| | | | NO: 233) | NO: 250) | |
| 11 | PD-L1 | PYSLI (SEQ ID | ISSYYI | YISPYSSYTS | GDWWGAL (SEQ ID |
| | | NO: 203) | (SEQ ID | (SEQ ID | NO: 273) |
| | | | NO: 230) | NO: 251) | |
| 12 | PD-L1 | YWWPGSLI (SEQ | VSSYSI | SIYSYSGYTY | YQHLAL (SEQ ID NO: 274) |
| | | ID NO: 204) | (SEQ ID | (SEQ ID | |
| | | | NO: 234) | NO: 252) | |
| 13 | PD-L1 | SSWEPV (SEQ ID | IYYYYI | SISPYSGYTS | SPSIVWAWHWQYGPGF |
| | | NO: 205) | (SEQ ID | (SEQ ID | (SEQ ID NO: 275) |
| | | | NO: 235) | NO: 147) | |
| 14 | PD-L1 | SSSSLI (SEQ ID | FSSSSI (SEQ | SISSSSGSTS | SFMYGTWYPYGF (SEQ ID |
| | | NO: 50) | ID NO: 236) | (SEQ ID | NO: 276) |
| | | | | NO: 126) | |
| 15 | PD-L1 | SYSYLV (SEQ ID | FSSSSI (SEQ | SIYSSSGSTS | YWWAFHWESHSYQPSYGF |
| | | NO: 206) | ID NO: 236) | (SEQ ID | (SEQ ID NO: 277) |
| | | | | NO: 253) | |
| 16 | PD-L1 | SSSSLI (SEQ ID | FSSSSI (SEQ | YISSSSGSTS | SDFMLHWHWFGM (SEQ ID |
| | | NO: 50) | ID NO: 236) | (SEQ ID | NO: 278) |
| | | | | NO: 254) | |
| 17 | PD-L1 | AYYSPI (SEQ ID | LSYSYM | SISPSSSYTY | YYYWHYFWDAF (SEQ ID |
| | | NO: 195) | (SEQ ID | (SEQ ID | NO: 266) |
| | | | NO: 227) | NO: 244) | |
| 18 | PD-L1 | SSSSLI (SEQ ID | FSSSSI (SEQ | SISSSSGSTS | SHSYGSWYAYGL (SEQ ID |
| | | NO: 50) | ID NO: 236) | (SEQ ID | NO: 279) |
| | | | | NO: 126) | |
| 19 | PD-L1 | SMYYLI (SEQ ID | VSSSSI | SIYPYSGYTS | WEESRYWYKYYYQGGL |
| | | NO: 207) | (SEQ ID | (SEQ ID | (SEQ ID NO: 280) |
| | | | NO: 95) | NO: 255) | |
| 20 | PD-L1 | SWPGYPI (SEQ ID | FSYSSI | SIYPSYGSTY | NWSGYLAM (SEQ ID |
| | | NO: 208) | (SEQ ID | (SEQ ID | NO: 281) |
| | | | NO: 237) | NO: 136) | |
| 21 | PD-L1 | SSSSLI (SEQ ID | FSSSSI (SEQ | SISSSSGSTS | SQSYGSWYAYGL (SEQ ID |
| | | NO: 50) | ID NO: 236) | (SEQ ID | NO: 282) |
| | | | | NO: 126) | |
| 22 | PD-L1 | SSSSLI (SEQ ID | FSSSSI (SEQ | SISSSSGSTS | TSYWEYWYWFGL (SEQ ID |
| | | NO: 50) | ID NO: 236) | (SEQ ID | NO: 283) |
| | | | | NO: 126) | |
| 23 | PD-L1 | DYFGLI (SEQ ID | LSYSSI | SIYPYYGSTY | HYGFAM (SEQ ID NO: 284) |
| | | NO: 209) | (SEQ ID | (SEQ ID | |
| | | | NO: 238) | NO: 149) | |
| 24 | PD-L1 | SSWWSPI (SEQ ID | ISSSSI (SEQ | SIYSYYGSTS | GYYSSYSSWYLYGSDSAI |
| | | NO: 210) | ID NO: 239) | (SEQ ID | (SEQ ID NO: 285) |
| | | | | NO: 145) | |
| 25 | PD-L1 | SWPGSPV (SEQ | VSSSSI | YIYSYSGSTY | GYFPAM (SEQ ID NO: 286) |
| | | ID NO: 211) | (SEQ ID | (SEQ ID | |
| | | | NO: 95) | NO: 256) | |
| 26 | PD-L1 | GGYWLV (SEQ ID | VSYSSI | SISSYYGSTY | HRYFAM (SEQ ID NO: 287) |
| | | NO: 212) | (SEQ ID | (SEQ ID | |
| | | | NO: 88) | NO: 257) | |
| 27 | PD-L1 | SSSSLI (SEQ ID | FSSSSI (SEQ | SISSSSGSTS | YGGYGEYFSWYPYGM |
| | | NO: 50) | ID NO: 236) | (SEQ ID | (SEQ ID NO: 288) |
| | | | | NO: 126) | |
| 28 | PD-L1 | YYHPI (SEQ ID | LSYYSI | SISPYYSYTS | SPWDPWAHHGHGI (SEQ ID |
| | | NO: 193) | (SEQ ID | (SEQ ID | NO: 264) |
| | | | NO: 225) | NO: 124) | |
| 29 | PD-L1 | SSSSLI (SEQ ID | FSSSSI (SEQ | SISSSSGSTS | PYWWSGQGYWGF (SEQ ID |
| | | NO: 50) | ID NO: 236) | (SEQ ID | NO: 289) |
| | | | | NO: 126) | |
| 30 | PD-LI | HYQPLI (SEQ ID | LSSSSI | SISPYSGYTS | GEGQQYKWSPYGL (SEQ ID |
| | | NO: 213) | (SEQ ID | (SEQ ID | NO: 290) |
| | | | NO: 240) | NO: 147) | |
| 31 | PD-L1 | PIWYEPI (SEQ ID | IYSSSI (SEQ | SISPYSGYTS | WYYAWHMGVKGYQGF |
| | | NO: 214) | ID NO: 71) | (SEQ ID | (SEQ ID NO: 291) |
| | | | | NO: 147) | |
| 32 | PD-L1 | SSSSLI (SEQ ID | LSSYSI | YISSYSGSTS | QEWYYGFGAYKYHWATGL |
| | | NO: 50) | (SEQ ID | (SEQ ID | (SEQ ID NO: 292) |
| | | | NO: 81) | NO: 258) | |
| 33 | PD-L1 | SWPQYPV (SEQ | FSYSSI | SIYSSYGSTS | WRSLAL (SEQ ID NO: 293) |
| | | ID NO: 215) | (SEQ ID | (SEQ ID | |
| | | | NO: 237) | NO: 259) | |
| 34 | PD-L1 | WNYALI (SEQ ID | FSSSSI (SEQ | SISSSSGSTS | ESYWWWSYWHLGL (SEQ |
| | | NO: 216) | ID NO: 236) | (SEQ ID | ID NO: 294) |
| | | | | NO: 126) | |
| 35 | PD-L1 | SSSSLI (SEQ ID | FSSSSI (SEQ | SISSSSGSTS | GDDHVYWWWFGM (SEQ |
| | | NO: 50) | ID NO: 236) | (SEQ ID | ID NO: 295) |
| | | | | NO: 126) | |
| 36 | PD-L1 | SSWWSPI (SEQ ID | ISSSSI (SEQ | SIYSYYGSTS | GYYSSYSSWYLYGSDSAI |
| | | NO: 210) | ID NO: 239) | (SEQ ID | (SEQ ID NO: 285) |
| | | | | NO: 145) | |
| 37 | PD-L1 | YNWSQLI (SEQ | FSYSYI | SIYPYSGYTS | DYSAYYAM (SEQ ID |
| | | ID NO: 217) | (SEQ ID | (SEQ ID | NO: 296) |
| | | | NO: 241) | NO: 255) | |
| 38 | PD-L1 | YHWPSELF (SEQ | LSSSSI | SIYSSYGYTS | QGWLAL (SEQ ID NO: 297) |
| | | ID NO: 218) | (SEQ ID | (SEQ ID | |
| | | | NO: 240) | NO: 260) | |
| 39 | PD-L1 | SGQYWPF (SEQ | VYSSSI | SISPSSSYTS | SYGEGSYTFWIWAGYGAL |
| | | ID NO: 219) | (SEQ ID | (SEQ ID | (SEQ ID NO: 298) |
| | | | NO: 87) | NO: 261) | |
| 40 | PD-L1 | SASWEPV (SEQ | IYYSSI | SISSYYGYTY | SYNYHYYTPYGF (SEQ ID |
| | | ID NO: 220) | (SEQ ID | (SEQ ID | NO: 299) |
| | | | NO: 242) | NO: 262) | |
| 41 | PD-L1 | SYYYLV (SEQ ID | VSSSSI | SISSSYGYTS | YPYEVSWTPYGM (SEQ ID |
| | | NO: 221) | (SEQ ID | (SEQ ID | NO: 300) |
| | | | NO: 95) | NO: 250) | |
| 42 | PD-L1 | SSSSLI (SEQ ID | FSSSSI (SEQ | SISSSSGSTS | AEWYLHFEQGFGF (SEQ ID |
| | | NO: 50) | ID NO: 236) | (SEQ ID | NO: 301) |
| | | | | NO: 126) | |
| 43 | PD-L1 | SYPHSLI (SEQ ID | VYSSYI | SISSYSGSTS | YYYKYMAM (SEQ ID |
| | | NO: 222) | (SEQ ID | (SEQ ID | NO: 302) |
| | | | NO: 86) | NO: 263) | |
| 44 | PD-L1 | SYSYLV (SEQ ID | VSSSSI | SIYSSSGYTS | YWWPFHWESHSYQPSYGF |
| | | NO: 206) | (SEQ ID | (SEQ ID | (SEQ ID NO: 303) |
| | | | NO: 95) | NO: 122) | |
| 45 | PD-L1 | SSYSLF (SEQ ID | VYYSSI | SISSYYGYTY | SYNYQGDNWHEYYPSGL |
| | | NO: 223) | (SEQ ID | (SEQ ID | (SEQ ID NO: 304) |
| | | | NO: 89) | NO: 262) | |
| 46 | PD-L1 | YNLSLV (SEQ ID | IYSSSI (SEQ | SIYSSSGYTS | YYGYGM (SEQ ID NO: 305) |
| | | NO: 224) | ID NO: 71) | (SEQ ID | |
| | | | | NO: 122) | |

TABLE 4
Additional VH CDRs for a VH binder.

| | E3 | | | |
|---|---|---|---|---|
| Con- | ligase | HC-CDR1 | HC-CDR2 | HC-CDR3 |
| struct | antigen | | | |
| 1 | RNF43 | AFSYYDY | RIYPYSSYTS | YSPGYYPFRGWGGM |
| | | (SEQ ID | (SEQ ID | (SEQ ID NO: 314) |
| | | NO: 306) | NO: 311) | |
| 2 | RNF43 | DFYSSDD | RIYSSYGSTY | YAWRPSGGYYSYAM |
| | | (SEQ ID | (SEQ ID | (SEQ ID NO: 315) |
| | | NO: 307) | NO: 312) | |
| 3 | RNF43 | AIYSYYYD | RIYSSYGSTY | GSYYFGYAF |
| | | (SEQ ID | (SEQ ID | (SEQ ID NO: 316) |
| | | NO: 308) | NO: 312) | |
| 4 | RNF43 | AIYYSYD | RIYSSYGSTY | WGWSYDPAGYAYAM |
| | | (SEQ ID | (SEQ ID | (SEQ ID NO: 317) |
| | | NO: 309) | NO: 312) | |
| 5 | RNF43 | RISSYYYD | RIYPYSGYTS | WYDSPGF |
| | | (SEQ ID | (SEQ ID | (SEQ ID NO: 318) |
| | | NO: 310) | NO: 313) | |

TABLE 5
Variable Domains for Surface Targets

| | | LC variable | HC variable |
|---|---|---|---|
| | | (VL) domain | (VH) domain |
| Construct | Target antigen | SEQ ID NO: | SEQ ID NO: |
| B1 | PD-L1 | 106 | 107 |
| B2 | HER2 | 108 | 109 |
| B3 | EGFR | 110 | 111 |
| B4 | CTLA-4 | 112 | 113 |
| B5 | CDCP1 | 114 | 115 |
| — | MMP14-binder 1 | 329 | 328 |
| — | MMP14-binder 2 | 331 | 330 |

### Example 2

This Example describes experiments performed to test each of the following types of constructs: bispecific IgG, bispecific IgG with a single chain Fab on one arm, and a Fab-scFV fusion.

### Western Blot

Cell lines MDA-MB-231, HCC827, H460, and T24 were tested for PD-L1 degradation by Western blot, following the 3-day process described below.

At day one, cells at ˜60-70% confluency in a 6 well plate were dosed with differing concentrations of bispecific antibody in 1 mL of fresh growth media. The cells were then left undisturbed for set length of time (24 hours).

At day two, and 24 hours after incubation with bispecific antibodies, the samples were considered ready for Western blot analysis. To do so, the cell culture media was aspirated, and the cells were washed with cold PBS. The cells were subsequently lifted with Gibco© Versene Solution and spun down. Then, the supernatant was removed and the cell pellets were individually resuspended in 140 μL of RIPA lysis buffer+cOmplete™ protease inhibitor cocktail (Millipore Sigma, #11836170001) and transferred to an Eppendorf tube. The resuspended cells were then incubated at 4° C. with lysis buffer for 30 minutes. (Lysis buffer: 5M NaCl (3 mL), 1 M Tris-HCl (5 mL, pH 8.0), Nonidet™ P-40 (1 mL), 10% sodium deoxycholate (5 mL), 10% SDS (1 mL), ddH₂O (qs to 100 mL)). Cell lysates were then spun down, 15000 g, 10 minutes, 4° C., 100 μL of soluble fraction was taken and protein levels normalized using a bicinchoninic acid assay (BCA assay, also known as the Smith assay). Diluted lysates were added to 20 μL LDL buffer+2 μL BME, and the solutions boiled for 10 minutes. Lysates were ran on an SDS page gel (200V, 37 minutes), and the gel was blocked with a solution of 20% ethanol. Blocked gel was transferred to a polyvinylidene fluoride (PVDF) membrane using the iBlot2© platform. Membrane was blocked using the manufacturer's blocking buffer for 60 minutes. The primary antibodies were added in 7.5 mL blocking buffer+0.2% Tween® 20. The ratio for anti-PD-L1 was 1:1000, and the ratio for anti-tubulin was 1:2000. Finally, the membrane was gently shaken overnight at 4° C. in a black box.

At day three, the overnight buffer was removed, and the membrane was rinsed with 1×TBS-T (0.1% Tween® 20), and then covered with 1×TBS-T (0.1% Tween® 20) and shaken at RT for 5 minutes. The wash solution was poured off, and the rinsing process repeated 3 additional times. The secondary antibodies were diluted in 8 mL of blocking buffer+0.2% Tween® 20 (160 μL)+0.01% SDS (8 μL). Two secondary antibodies were used: Goat-anti-Rabbit (800 nm), and Goat-anti-Mouse (680 nm). Membrane was incubated in the dark with secondary antibodies for 1 hour at RT with gentle shaking. Following this incubation, the membrane was rinsed with 1×TBS-T (0.1% Tween® 20), and covered with 1×TBS-T (0.1% Tween® 20) and shaken at RT for 5 minutes. The wash solution was poured off, and the membrane rinsed three additional times. A final rinse with membrane with 1×PBS was done to remove residual Tween® 20, and the membranes were imaged on a Li-Cor® imaging system.

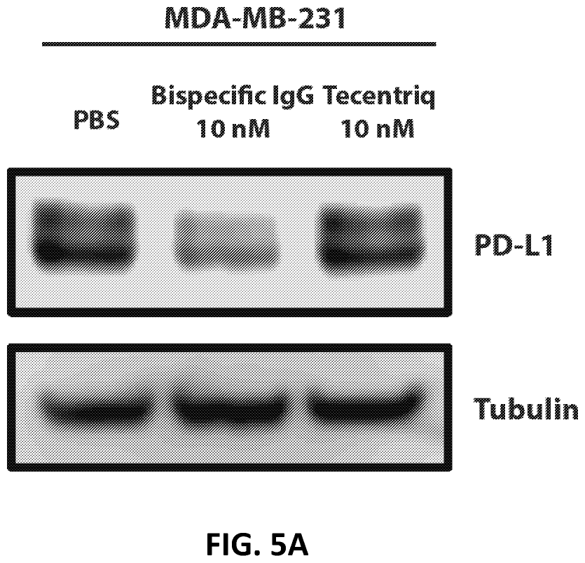

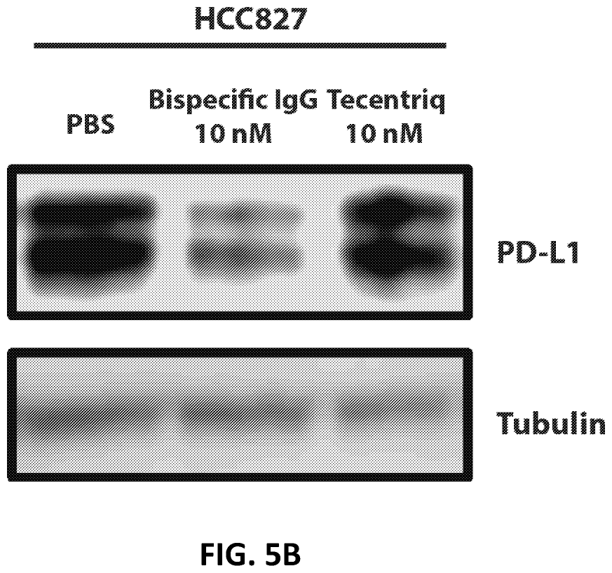

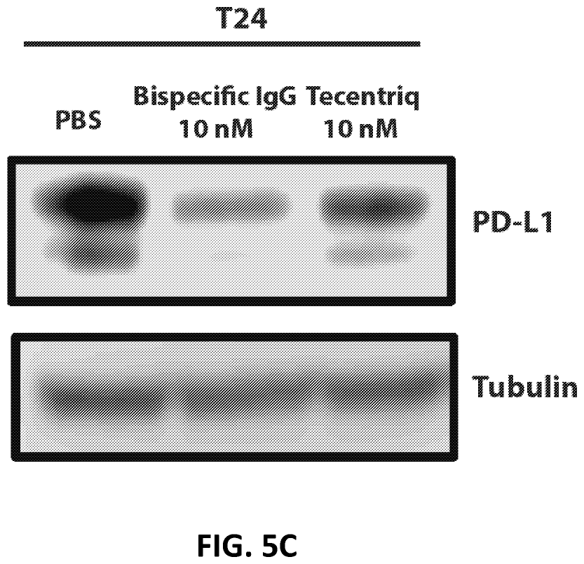

Exemplary Western blot results for the effects on levels of PD-L1 in either the MDA-MB-231, HCC827, or T24 cell line, after treatment for 24 hours with either the tested bispecific IgG or atezolizumab (Tecentriq®, both at 10 nM in solution), are shown in FIGS. 5A, 5B, and 5C, respectively. Briefly, the tested bispecific IgG was able to degrade PD-L1 in these three different clinically relevant cell lines (MDA-MB-231, HCC827, or T24), whereas atezolizumab resulted in little or no degradation.

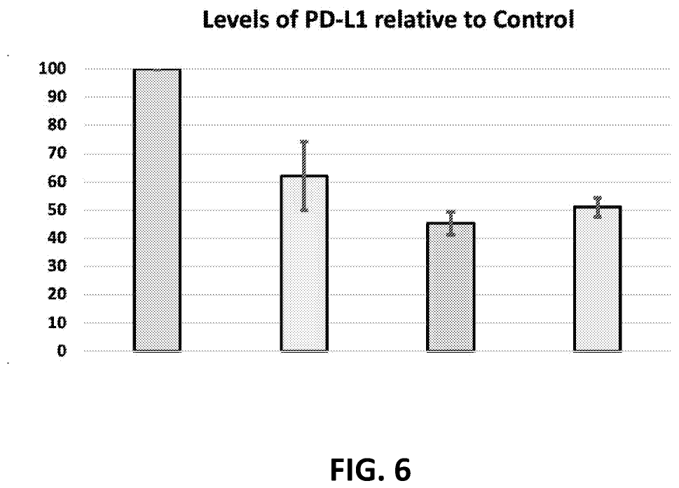

Additionally, FIG. 6 shows the effects of bispecific RNF43-PD-L1 IgGs on degrading PD-L1 from the triple negative breast cancer cell line, MDA-MB-231. Each of the bars from left to right represents: PBS control, 10 nM of the construct with RNF43 A5 (SEQ ID NOs.: 332 and 333), 10 nM—the Fab construct of RNF43 A4 (SEQ ID NOs.: 322 and 323), and 10 nM—the Fab construct of RNF43 A6 (SEQ ID NOs.: 324 and 325). All constructs were bispecific IgG's with one arm targeting RNF43 and the other binding to PD-L1 with Tecentriq as the binding arm. PD-L1 binding variable domains were the same in all the constructs and had SEQ ID NOs: 106 and 107. The Western blot was done in the same way as described in Example 2 of the present disclosure.

### Flow Cytometry

Cell lines were tested with success for PD-L1: MDA-MB-231, HCC827, H460, and T24, by flow cytometry, following the 2-day process described below.

At day one, cells at ˜60-70% confluency in a 6 well plate were dosed with differing concentrations of bispecific antibody in 1 mL of fresh growth media. The cells were then left undisturbed for set length of time (24 hours).

At day two, and 24 hours after incubation with bispecific antibodies, the samples were considered ready for Western blot analysis. To do so, the cell culture media was aspirated, and the cells were washed with cold PBS. The cells were subsequently lifted with Gibco® Versene Solution and spun down. Then, the supernatant was removed and the cell pellets were individually washed with cold 1×PBS. Then, the cells were then blocked with PBS+3% BSA, and biotinylated antibodies were added to samples and incubated at 4° C. for 30 minutes with shaking. The cells were then cells washed three times with PBS+3% BSA. Alexa Fluor® 647 streptavidin (ThermoFisher Scientific) was then added and cells incubated at 4° C. for 30 minutes with shaking. Cells were washed three times with PBS+3% BSA. Finally, the cells were resuspended in 200 μL PBS and run on the flow cytometer.

Flow cytometry results showed that the tested bispecific IgG was able to degrade PD-L1 in these three different clinically relevant cell lines (MDA-MB-231, HCC827, and T24), whereas atezolizumab resulted in little or no degradation.

### Example 3

### Engineered Transmembrane Proteins

This Example describes experiments performed in relation to the synthesis and test of an RNF43 engineered transmembrane protein in degrading a reporter construct.

### Transfection and Synthesis

All DNA fragments were purchased from IDT, and were assembled using Gibson Cloning. DNA fragments with 30 bp overlaps were incubated with cut vector pFUSE vector and Gibson master mix for 30 minutes at 50° C. An engineered transmembrane protein was designed based on RNF43 and an anti-GFP scFab. The anti-GFP scFab sequence is provided in SEQ ID NO: 2 (light chain) and SEQ ID NO: 4 (heavy chain), with the linking domain set provided in SEQ ID NO: 3. A short linker (SEQ ID NO: 5) connects the scFab domain to the RNF43 domain (SEQ ID NO: 6). The full sequence is set forth in SEQ ID NO: 1.

The reporter construct was assembled from a GFP domain (SEQ ID NO: 8), a transmembrane/linker domain (SEQ ID NO: 9), and a nanoluciferase domain (SEQ ID NO: 10). The full sequence of the reporter construct is provided in SEQ ID NO: 7.

Gibson products were transformed into XL10 competent cells via heat shock. Transformed cells were allowed to recover for 1 hour at 37° C. Recovered cells were plated on LB/Carbenicillin plates overnight at 37° C.

On day 2, Single colonies over the overnight plates were picked and added to 5 mL low salt LB/Carbenicillin and incubated at 37° C. until confluency. When the cells were at confluency the DNA was mini-prepped, and the sequences verified.

For the synthesis of the RNF43 engineered transmembrane protein, Hek293 or HeLa cells were transiently transfected with both reporter GFP-Nanoluc construct and RNF43 engineered transmembrane protein using the TransIT©-293 transfection reagent. Cells in a 6 well plate were allowed to grow to 60-70% confluency. DNA (2.5 μg) was incubated with 7.5 μL of TransIT-293 reagent in Opti-mem for 20 minutes at RT, and DNA-TransIt mixture was added to cells.

Hek293 FLP/IN cells were used to make a stable cell line expressing GFP-NanoLuc construct; for this cell line, the below experiment (nanoluciferase readout) was done with transient transformation of RNF43 fusion into this cell line, as the GFP-NanoLuc was already present. Transient transfections were done in a 6 well plate with the cells at ˜60% confluency.

### Nanoluciferase Readout

For the nanoluciferase readout mentioned above, and 24 hours after transfection with the appropriate construct or constructs, the transfected cells were split into a 96 well plate, and left undisturbed for 24 hours. Nano-Glo© reagents were thawed at RT and mixed 1:50. An equal volume of reagent was added to the cells (100 μL), and the cells were shaken at RT for 10 minutes. Finally, chemiluminescence was read on a plate reader. The nanoluciferase signal showed a significant decrease in reporter protein upon addition of anti-GFP-RNF43 fusion, compared to a negative control.

### Confocal Microscopy

For confocal microscopy and subsequent confocal fluorescent imaging, the cells were transfected as described above and incubated for 48 hours. After 48 hours, the cells were plated onto a glass bottom petri dish for 12 hours prior to imaging. Prior to imaging, the cell media was replenished, and LysoTracker® was added to the solution. The cells were fixed using 4% PFA, and permeabilized using 0.5% Triton™-X in PBS. DAPI (4′,6-diamidino-2-phenylindole) was incubated with permeabilized cells. Finally, the cells washed three times with PBS and imaged on a 100× spinning dish confocal microscope.

Confocal microscopy showed that the soluble GFP Fab alone had no effect on the localization of the GFP reporter, while anti-GFP-RNF43 engineered transmembrane protein resulted in the internalization and lysosomal aggregation of the GFP reporter. These data suggested that RNF43 can be used to induce protein degradation of endogenous proteins.

### Example 4

This experiment was designed to generate an AAV transfection vector for inserting an engineered transmembrane protein into a target cell.

An AAV transfer plasmid is constructed by placing a gene expressing scFab-E3 engineered transmembrane protein under CAG, EF1, or a tissue-specific promoter. HEK293T cells are transfected with AAV helper plasmid (pHelper), Rep-Cap plasmid (pAAV-RC1 or pAAV-RC9), and AAV transfer plasmid in a 1:1:2 ratio. The cells are incubated at 37° C. under 5% CO₂ for 3 days. Cells are then harvested and lysed with sonication in PBS buffer supplemented with 0.001% Pluronic® acid and 200 mM NaCl. Cell debris is pelleted at 3,200×g for 15 minutes at 4° C., and the supernatant is transferred in another tube. Benzonase (50 units/mL) is added to the supernatant, which is further incubated at 37° C. for 45 minutes. The supernatant is clarified by centrifugation at 2,400 g at 4° C. for 10 minutes. The recombinant AAV is subsequently purified by two rounds of iodixanol gradient ultracentrifugation (15%, 25%, 40% and 60% iodixanol diluted in PBS-MK gradient buffer) and the 40% fractions are pooled and desalted using a MWCO 100 kDa centrifugal concentrator device. The desalted AAV is then stored at −80° C. in PBS supplemented with 0.001% Pluronic® acid and 200 mM NaCl.

For the production of exosome-associated AAV (exoAAV), a stable cell line of HEK293T that would overexpress CD9-GFP is constructed by lentiviral transduction. The HEK293TCD9-GFP cells are transfected with AAV helper plasmid (pHelper), Rep-Cap plasmid (pAAV-RC1 or pAAV-RC9), and AAV transfer plasmid, in a 1:1:2 ratio. The cells are then incubated in exosome-depleted medium at 37° C. under 5% C02 for 3 days, after which the cell medium is collected and depleted at 300×g for 5 minutes and 1000 g for 10 minutes. The supernatant is centrifuged at 20,000 g for 1 hour at 15° C., then collected and centrifuged again at 100,000×g for 1.5 hours at 15° C. The final exoAAV product is then stored at 4° C.

### In Vitro Targeting of AAV

A cell line of Hela cells that stably expresses the GFP-nanoluciferase (GFP-Nluc, SEQ ID NO: 7) reporter gene is constructed via the Flp-In™ System recombination system (ThermoFisher Scientific). Hela^{GFP-Nluc} cells are seeded at 50,000 cells/well in 96 well plates 24 hours before transduction. The cells are transduced with 108 genomic copies of standard AAV or exoAAV overnight at 37° C. under 5% CO₂. The culture medium is replaced with Dulbecco/Vogt modified Eagle's minimal essential medium (DMEM) with 10% FBS, and the cells are further incubated at 37° C. under 5% CO₂. Luciferase assay and flow analysis are performed 48 hours post-transduction with the same procedures as above.

### Example 5

### Kinetic Requirement

This Example provides additional data on using a bispecific antibody to degrade PD-L1 by recruiting RNF43. The results were slightly different from what one would expect it to be. The conclusion is that there is an affinity requirement for each component of the bispecific antibody that dictates how good of a degrader it is. However, it was originally hypothesized that binding really tight might not be ideal and that a slightly weaker binder might improve the turnover of the degradation process and thus increase the level of degradation. Surprisingly, it was observed that a tight binder with a slow off-rate was actually needed to induce degradation.

An Alanine scan of important residues involved in binding to PD-L1 was performed on the PD-L1 binding component, Tecentriq. The mutants were expressed as Fabs to measure their kinetic parameters (Kd, Kon, Koff). Then, they were turned into bispecific antibodies with the other half being the anti-RNF43 A5 construct. Next, a degradation experiment was performed with 10 nM bispecific IgG on MDA-MB-231 cells, using the same protocol as described in Example 2 above and with western blot to quantify degradation amounts. With these data, the degradation levels vs the different kinetic parameters were plotted. The data shows that there is a correlation (R²=0.67) between the Koff and degradation levels, meaning the slower the off rate the higher the degradation levels.

Next, similar experiments were performed for the RNF43 binder, by which an Alanine scan was performed, of the clone anti-RNF43 A5. Alanine mutants of the CDR H3 were made. All but 2 clones completely ablated binding, but 2 maintained some binding. These 2 mutants which maintained some binding had affinities for RNF43 of 40 and 125 nM (S113A and F115A respectively). They were made into bispecific IgG's using the WT PD-L1 binder with SEQ ID NOs: 106 and 107 and repeated the degradation experiment from above. This time, degradation was only seen in the case of the WT RNF43 binder, which has an affinity of 12.5 nM, and no change to PD-L1 levels when the binding to RNF43 was reduced to 40 or 125 nM. Again, this data supports the idea that a tight binder is required to each side of the bispecific antibody.

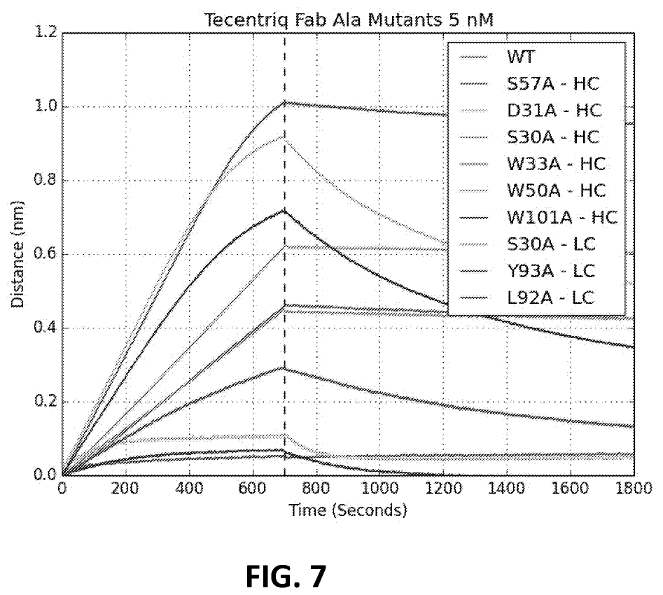

FIG. 7 shows a combined bio-layer interferometry (BLI) graphs of each Ala mutant. The kinetic parameters for each of the alanine mutants in the Bio-layer interferometry (BLI) graph in FIG. 7 are shown in Table 6 below.

TABLE 6
Kinetic parameters for each of the alanine mutants in
the Bio-layer interferometry (BLI) graph in FIG. 7.

| Mutant | Kd (nM) | Kon | Koff | X\hat{ }2 | R\hat{ }2 |
|---|---|---|---|---|---|
| WT | 0.385 | 1.48E+05 | 5.68E−05 | 0.6105 | 0.9988 |
| SS7A HC | 3.91 | 1.52E+04 | 5.95E−05 | 0.1385 | 0.9984 |
| D31A HC | 1.94 | 2.78E+05 | 5.40E−04 | 2.8417 | 0.9892 |
| S30A HC | 1.4 | 4.36E+04 | 6.09E−05 | 1.045 | 0.9983 |
| W33A HC | — | — | — | — | — |
| W50A HC | 63.1 | 1.38E+05 | 8.69E−03 | 0.3838 | 0.9722 |
| W101A HC | 458 | 1.10E+04 | 5.02E−03 | 0.1102 | 0.989 |
| S30A LC | 1.23 | 8.48E+04 | 1.05E−04 | 2.129 | 0.9969 |
| Y93A LC | 2.59 | 2.80E+05 | 7.24E−04 | 0.2413 | 0.9964 |
| L92A LC | 2.8 | 2.26E+05 | 6.32E−04 | 1.7696 | 0.9888 |

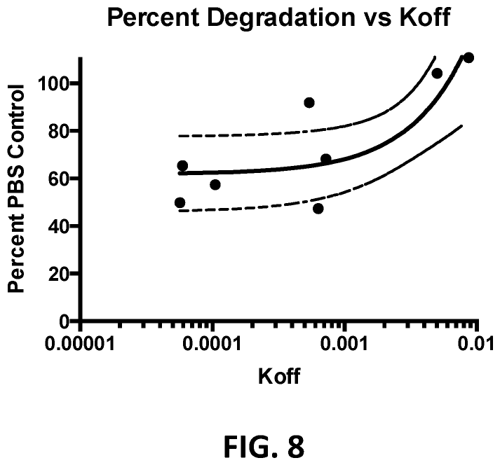

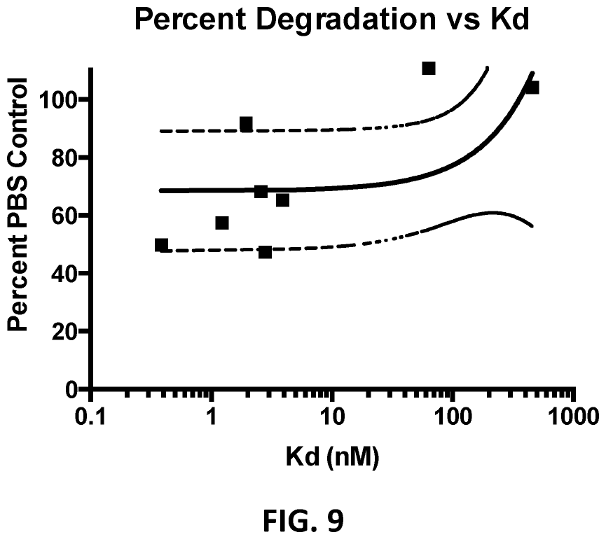

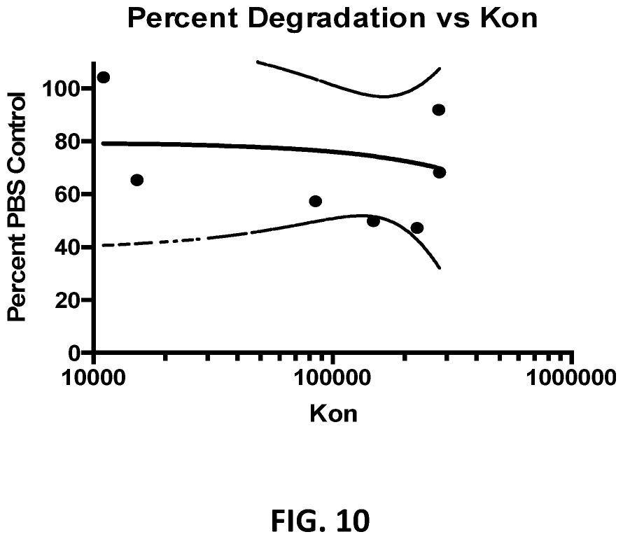

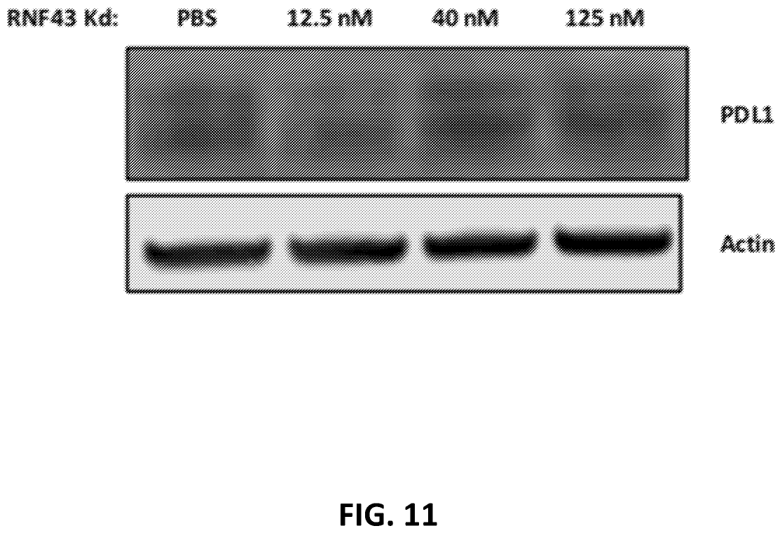

FIG. 8 shows the correlation between percent degradation vs Koff. The slower the off rate, the higher the degradation. Further, FIG. 9 shows the correlation between percent degradation vs Kd. As shown, there was a slight correlation, meaning the tighter the binder, the higher the degradation. FIG. 10 shows that there was no correlation between percent degradation vs Kon. FIG. 11 shows the Western blot of anti-RNF43 Alanine mutants. The mutants are labelled by their Kd's to RNF43. 12.5 nM is the WT RNF43 A5, 40 nM is S113A and 125 nM is F115A. This shows that after 24 hour treatment of bispecific binding agent at 10 nM, only the tightest binding anti-RNF43 construct degrades PD-L1.

### Example 6

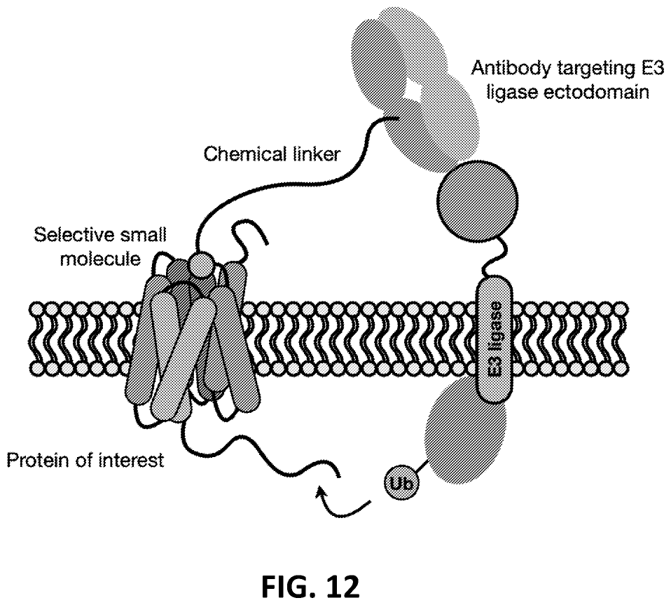

Provided herein is an exemplary method for small molecule conjugation with Fab constructs. These data suggest that an immunoconjugate comprising a binding agent of the present disclosure can be recruited to the target and induce its degradation. An exemplary illustration of the antibody-drug conjugates disclosed herein is provided in FIG. 12.

Cell lines. Cell lines were grown and maintained in T75 (Thermo Fisher Scientific) flasks at 37° C. and 5% C02. MOLT-4 CCR5+ cells were grown in RPMI-1640 supplemented with 10% fetal bovine serum (FBS) and 2% geneticin. MOLT-4 CCR5+ cells were obtained from the NIH AIDS Reagent Program.

Antibody cloning, expression, and purification. Anti-RNF43 Fab LC S7M single mutation was introduced using Gibson Assembly. Fabs were expressed in E. coli C43(DE3) Pro+ using an optimized autoinduction media and purified by Protein A affinity chromatography. (Hornsby, M. et al. A High Through-put Platform for Recombinant Antibodies to Folded Proteins. Mol. Cell. Proteomics 14, 2833-2847 (2015).) Purity and integrity of Fabs were assessed by SDS-PAGE and intact mass spectrometry. The light chain and heavy chain sequences for the anti-RNF43 Fab used for antibody-drug conjugate are set forth in SEQ ID NOs: 326 and 327, respectively.

Synthesis of DBCO-CGS21680. Commercially available CGS21680 (Cayman Chemical, 17126, 5 mg, 0.01 mmol) was added to 1.5 equivalents of 1-[Bis(dimethylamino)methylene]-1H-1,2,3-triazolo[4,5-b]pyridinium 3-oxide hexafluorophosphate (HATU, 6 mg, 0.015 mmol) and 4 equivalents of 4 equivalents N,N-diisopropylethylamine (7 μL, 0.04 mmol) in 2 mL of dimethylformamide and stirred at room temperature for 10 min. Then, 1.5 equivalents DBCO-PEG4-amine (BroadPharm, BP-23958, 5 mg, 0.015 mmol) was added to reaction flask and stirred at room temperature overnight. The reaction mixture was concentrated under reduced pressure. Crude product was purified by high performance liquid chromatography (HPLC). The final product was lyophilized and isolated as a pale yellow powder (4.8 mg, 48% yield). ESI-HRMS calculated [M+H⁺]=1005.48; found 1005.54.

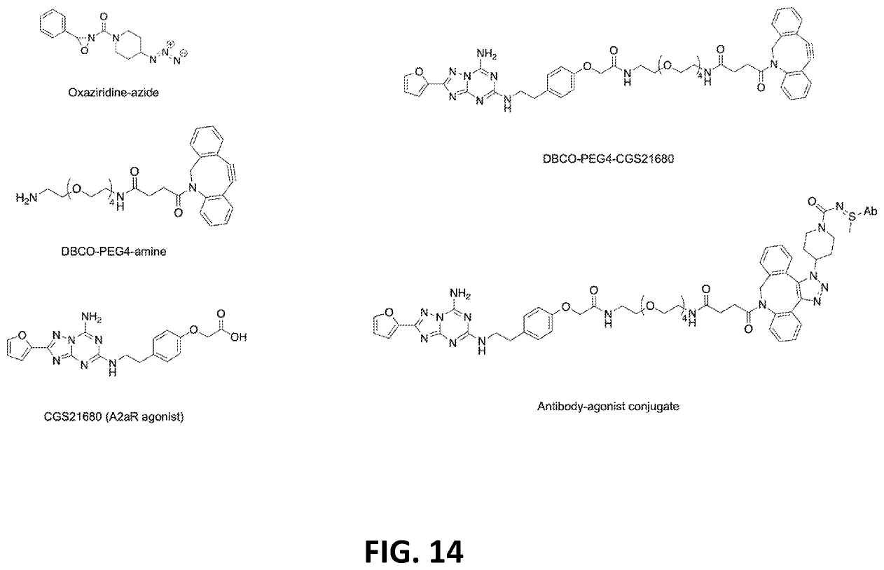

Conjugation of engineered anti-RNF43 Fab with oxaziridine and DBCO-CGS21680. An exemplary illustration of the conjugation process is provided in FIG. 13. For conjugation with oxaziridine, 50 μM Fab was incubated with 5 molar equivalents of oxaziridine azide for 30 min at room temperature in phosphate-buffer saline (PBS). (A. H. Christian et al., A physical organic approach to tuning reagents for selective and stable methionine bioconjugation. J. Am. Chem. Soc. 141, 12657-12662 (2019).) The reaction was quenched with 10 molar equivalents methionine. The antibody was buffer exchanged into PBS and desalted using a 0.5-mL Zeba 7-kDa desalting column (Thermo Fisher Scientific). Then, 10 molar equivalents of DBCO-CGS21680 was added and incubated at room temperature overnight. The agonist-labeled conjugate was desalted using the 0.5-mL Zeba 7-kDa desalting column to remove excess DBCO-CGS21680. Full conjugation at each step was monitored by intact mass spectrometry using a Xevo G2-XS Mass Spectrometry (Waters). Some exemplary small molecules used in the conjugation are provided in FIG. 14. This is not meant to be an exhaust list of small molecules that can be used for conjugation, and one skilled in the art would understand what alternative small molecules can be conjugated to the antigen-binding agents provided in the present disclosure based on the utility.

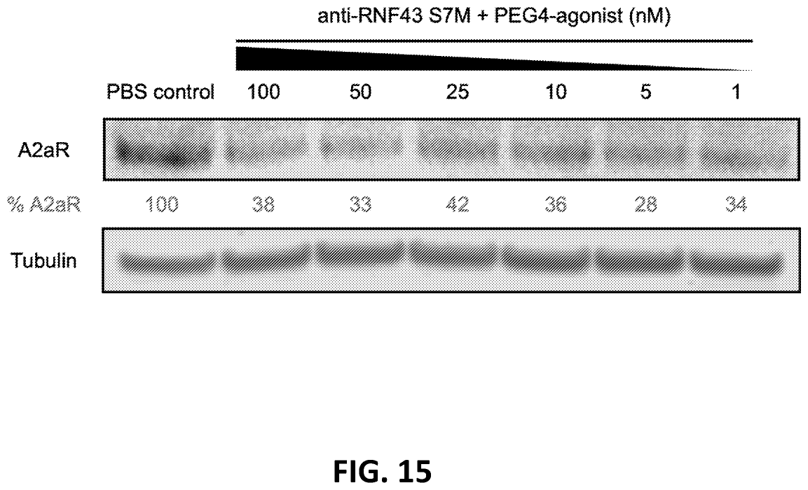

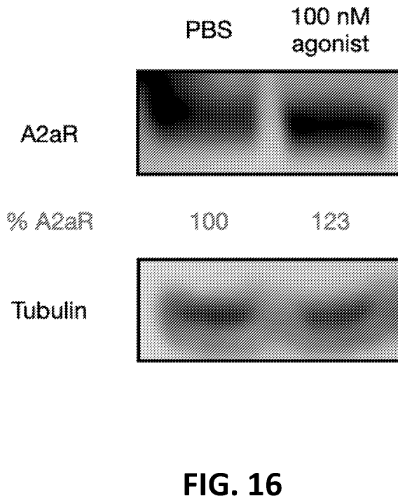

Degradation assays. Cells at 1 million cells/mL were treated with antibody-drug conjugate, agonist, or antagonist in complete growth medium. After 24 hrs, cells were pelleted by centrifugation (300×g, 5 min, 4° C.). Cell pellets were lysed with RIPA buffer containing cOmplete™ mini protease inhibitor cocktail on ice for 40 min. Lysates were spun at 16,000×g for 10 min at 4° C. and protein concentrations were normalized using BCA assay. 4× NuPAGE LDS sample buffer and 2-mercaptoethanol (BME) was added to the lysates. Equal amounts of lysates were loaded onto a 4-12% Bis-Tris gel and ran at 200V for 37 min. The gel was incubated in 20% ethanol for 10 min and then transferred onto a polyvinylidene difluoride (PVDF) membrane. The membrane was blocked in PBS with 0.1% Tween20+5% bovine serum albumin (BSA) for 30 min at room temperature with gentle shaking. Membranes were co-incubated overnight with rabbit-anti-A2aR (Abcam, ab3461, 1:1000) and mouse-anti-tubulin (Cell Signaling Technologies, DM1A, 1:1600) at 4° C. with gentle shaking in PBS+0.2% Tween20+5% BSA. Membranes were washed four times with tris-buffered saline (TBS)+0.1% Tween20 and then co-incubated with HIRP-anti-rabbit IgG (Cell Signaling Technologies, 7074S, 1:2000) and 680RD goat anti-mouse IgG (LI-COR, 926-68070, 1:10000) in PBS+0.2% Tween20+5% BSA for 1 hr at room temperature. Membranes were washed four times with TBS+0.1% Tween20, then washed with PBS. Membranes were first imaged using an OdysseyCLxImager (LI-COR). SuperSignal West Pico PLUS Chemiluminescent Substrate was then added and image using a ChemiDoc Imager (BioRad). Band intensities were quantified using Image Studio Software (LI-COR). Exemplary results are shown in FIGS. 15 and 16. In particular, FIG. 15 shows the degradation of adenosine 2a receptor (A2aR) in MOLT-4 CCR5+ cells after 24 hr treatment, and FIG. 16 shows the A2aR levels after 24 hr treatment of CGS21680 (agonist) or ZM241385 (antagonist). These data suggest that RNF43 can be recruited to A2aR using an immunoconjugate to induce its degradation at a concentration of 1 nM after 24 hours (FIG. 15). FIG. 16 is a control demonstrating that treatment with just the small molecule (100 nM agonist) without it being conjugated to the anti-RNF43 fab has no effect on A2aR levels.

Other targets include, without limitation, CXCR4, CCR5, Smoothened, CCR2, CCR9, Proteinase activated receptor 1 (PAR1), PAR2, Mu opioid receptor, Delta opioid receptor, Kappa opioid receptor, and Neurokinin receptor 1.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

INFORMAL SEQUENCE LISTING

| SEQ | | |
|---|---|---|
| ID | | |
| NO | Sequence | Description |
| 1 | DIQMTQSPSSLSASVGDRVTITCRASQSVSSAVAWYQQKPGKAPKLLIYSA | RNF43-antiGFP |
| | SSLYSGVPSRFSGSRSGTDFTLTISSLQPEDFATYYCQQSWGLITFGQGTKV | scFab construct |
| | EIKRTVAAPSVFIFPPSDSQLKSGTASVVCLLNNFYPREAKVQWKVDNALQ | |
| | SGNSQESVTEQDSKDSTYSLSSTLTLSKADYEKHKVYACEVTHQGLSSPVT | |
| | KSFNRGECGGSSGSGSGSTGTSSSGTGTSAGTTGTSASTSGSGSGGGGGSG | |
| | GGGSAGGTATAGASSGSEVQLVESGGGLVQPGGSLRLSCAASGFNISYYSI | |
| | HWVRQAPGKGLEWVASIYPYYSSTSYADSVKGRFTISADTSKNTAYLQMN | |
| | SLRAEDTAVYYCARAGWVASSGMDYWGQGTLVTVSSASTKGPSVFPLAP | |
| | SSKSTSGGTAALGCLVKDYFPEPVTVSWNSGALTSGVHTFPAVLQSSGLYS | |
| | LSSVVTVPSSSLGTQTYICNVNHKPSNTKVDKKVEPKSCDKTHTGGGGSG | |
| | GGGSGFGRTGLVLAAAVESERSAEQKAIIRVIPLKMDPTGKLNLTLEGVFA | |
| | GVAEITPAEGKLMQSHPLYLCNASDDDNLEPGFISIVKLESPRRAPRPCLSL | |
| | ASKARMAGERGASAVLFDITEDRAAAEQLQQPLGLTWPVVLIWGNDAEK | |
| | LMEFVYKNQKAHVRIELKEPPAWPDYDVWILMTVVGTIFVIILASVLRIRC | |
| | RPRHSRPDPLQQRTAWAISQLATRRYQASCRQARGEWPDSGSSCSSAPVC | |
| | AICLEEFSEGQELRVISCLHEFHRNCVDPWLHQHRTCPLCMFNITEGDSFSQ | |
| | SLGPSRSYQEPGRRLHLIRQHPGHAHYHLPAAYLLGPSRSAVARPPRPGPFL | |
| | PSQEPGMGPRHHRFPRAAHPRAPGEQQRLAGAQHPYAQGWGLSHLQSTS | |
| | QHPAACPVPLRRARPPDSSGSGESYCTERSGYLADGPASDSSSGPCHGSSSD | |
| | SVVNCTDISLQGVHGSSSTFCSSLSSDFDPLVYCSPKGDPQRVDMQPSVTSR | |
| | PRSLDSVVPTGETQVSSHVHYHRHRHHHYKKRFQWHGRKPGPETGVPQS | |
| | RPPIPRTQPQPEPPSPDQQVTRSNSAAPSGRLSNPQCPRALPEPAPGPVDASS | |
| | ICPSTSSLFNLQKSSLSARHPQRKRRGGPSEPTPGSRPQDATVHPACQIFPHY | |
| | TPSVAYPWSPEAHPLICGPPGLDKRLLPETPGPCYSNSQPVWLCLTPRQPLE | |
| | PHPPGEGPSEWSSDTAEGRPCPYPHCQVLSAQPGSEEELEELCEQAV | |
| 354 | SDIQMTQSPSSLSASVGDRVTITCRASQSVSSAVAWYQQKPGKAPKLLIYS | RNF43-antiGFP |
| | ASSLYSGVPSRFSGSRSGTDFTLTISSLQPEDFATYYCQQSWGLITFGQGTK | scFab construct, |
| | VEIKRTVAAPSVFIFPPSDSQLKSGTASVVCLLNNFYPREAKVQWKVDNAL | alternative |
| | QSGNSQESVTEQDSKDSTYSLSSTLTLSKADYEKHKVYACEVTHQGLSSPV | |
| | TKSFNRGECGGSSGSGSGSTGTSSSGTGTSAGTTGTSASTSGSGSGGGGGS | |
| | GGGGSAGGTATAGASSGSEVQLVESGGGLVQPGGSLRLSCAASGFNISYYS | |
| | IHWVRQAPGKGLEWVASIYPYYSSTSYADSVKGRFTISADTSKNTAYLQM | |
| | NSLRAEDTAVYYCARAGWVASSGMDYWGQGTLVTVSSASTKGPSVFPLA | |
| | PSSKSTSGGTAALGCLVKDYFPEPVTVSWNSGALTSGVHTFPAVLQSSGLY | |
| | SLSSVVTVPSSSLGTQTYICNVNHKPSNTKVDKKVEPKSCDKTHTGGGGSG | |
| | GGGSGFGRTGLVLAAAVESERSAEQKAIIRVIPLKMDPTGKLNLTLEGVFA | |
| | GVAEITPAEGKLMQSHPLYLCNASDDDNLEPGFISIVKLESPRRAPRPCLSL | |
| | ASKARMAGERGASAVLFDITEDRAAAEQLQQPLGLTWPVVLIWGNDAEK | |
| | LMEFVYKNQKAHVRIELKEPPAWPDYDVWILMTVVGTIFVIILASVLRIRC | |
| | RPRHSRPDPLQQRTAWAISQLATRRYQASCRQARGEWPDSGSSCSSAPVC | |
| | AICLEEFSEGQELRVISCLHEFHRNCVDPWLHQHRTCPLCMFNITEGDSFSQ | |
| | SLGPSRSYQEPGRRLHLIRQHPGHAHYHLPAAYLLGPSRSAVARPPRPGPFL | |
| | PSQEPGMGPRHHRFPRAAHPRAPGEQQRLAGAQHPYAQGWGLSHLQSTS | |
| | QHPAACPVPLRRARPPDSSGSGESYCTERSGYLADGPASDSSSGPCHGSSSD | |
| | SVVNCTDISLQGVHGSSSTFCSSLSSDFDPLVYCSPKGDPQRVDMQPSVTSR | |
| | PRSLDSVVPTGETQVSSHVHYHRHRHHHYKKRFQWHGRKPGPETGVPQS | |
| | RPPIPRTQPQPEPPSPDQQVTRSNSAAPSGRLSNPQCPRALPEPAPGPVDASS | |
| | ICPSTSSLFNLQKSSLSARHPQRKRRGGPSEPTPGSRPQDATVHPACQIFPHY | |
| | TPSVAYPWSPEAHPLICGPPGLDKRLLPETPGPCYSNSQPVWLCLTPRQPLE | |
| | PHPPGEGPSEWSSDTAEGRPCPYPHCQVLSAQPGSEEELEELCEQAV | |
| 2 | DIQMTQSPSSLSASVGDRVTITCRASQSVSSAVAWYQQKPGKAPKLLIYSA | anti-GFP Light chain |
| | SSLYSGVPSRFSGSRSGTDFTLTISSLQPEDFATYYCQQSWGLITFGQGTKV | domain of SEQ ID |
| | EIKRTVAAPSVFIFPPSDSQLKSGTASVVCLLNNFYPREAKVQWKVDNALQ | NO: 1 |
| | SGNSQESVTEQDSKDSTYSLSSTLTLSKADYEKHKVYACEVTHQGLSSPVT | |
| | KSFNRGEC | |
| 355 | SDIQMTQSPSSLSASVGDRVTITCRASQSVSSAVAWYQQKPGKAPKLLIYS | anti-GFP Light chain |
| | ASSLYSGVPSRFSGSRSGTDFTLTISSLQPEDFATYYCQQSWGLITFGQGTK | domain of SEQ ID |
| | VEIKRTVAAPSVFIFPPSDSQLKSGTASVVCLLNNFYPREAKVQWKVDNAL | NO: 354 |
| | QSGNSQESVTEQDSKDSTYSLSSTLTLSKADYEKHKVYACEVTHQGLSSPV | |
| | TKSFNRGEC | |
| 3 | GGSSGSGSGSTGTSSSGTGTSAGTTGTSASTSGSGSGGGGGSGGGGSAGGT | scFAB linker domain |
| | ATAGASSGS | of SEQ ID NO: 1 |
| 4 | EVQLVESGGGLVQPGGSLRLSCAASGFNISYYSIHWVRQAPGKGLEWVASI | Anti-GFP Heavy |
| | YPYYSSTSYADSVKGRFTISADTSKNTAYLQMNSLRAEDTAVYYCARAG | chain domain of SEQ |
| | WVASSGMDYWGQGTLVTVSSASTKGPSVFPLAPSSKSTSGGTAALGCLVK | ID NO: 1 |
| | DYFPEPVTVSWNSGALTSGVHTFPAVLQSSGLYSLSSVVTVPSSSLGTQTYI | |
| | CNVNHKPSNTKVDKKVEPKSCDKTHT | |
| 5 | GGGGSGGGGS | Linker domain of |
| | | SEQ ID NO: 1 |
| 6 | GFGRTGLVLAAAVESERSAEQKAIIRVIPLKMDPTGKLNLTLEGVFAGVAEI | RNF43 domain of |
| | TPAEGKLMQSHPLYLCNASDDDNLEPGFISIVKLESPRRAPRPCLSLASKAR | SEQ ID NO: 1 |
| | MAGERGASAVLFDITEDRAAAEQLQQPLGLTWPVVLIWGNDAEKLMEFV | |
| | YKNQKAHVRIELKEPPAWPDYDVWILMTVVGTIFVIILASVLRIRCRPRHSR | |
| | PDPLQQRTAWAISQLATRRYQASCRQARGEWPDSGSSCSSAPVCAICLEEF | |
| | SEGQELRVISCLHEFHRNCVDPWLHQHRTCPLCMFNITEGDSFSQSLGPSRS | |
| | YQEPGRRLHLIRQHPGHAHYHLPAAYLLGPSRSAVARPPRPGPFLPSQEPG | |
| | MGPRHHRFPRAAHPRAPGEQQRLAGAQHPYAQGWGLSHLQSTSQHPAAC | |
| | PVPLRRARPPDSSGSGESYCTERSGYLADGPASDSSSGPCHGSSSDSVVNCT | |
| | DISLQGVHGSSSTFCSSLSSDFDPLVYCSPKGDPQRVDMQPSVTSRPRSLDS | |
| | VVPTGETQVSSHVHYHRHRHHHYKKRFQWHGRKPGPETGVPQSRPPIPRT | |
| | QPQPEPPSPDQQVTRSNSAAPSGRLSNPQCPRALPEPAPGPVDASSICPSTSS | |
| | LFNLQKSSLSARHPQRKRRGGPSEPTPGSRPQDATVHPACQIFPHYTPSVAY | |
| | PWSPEAHPLICGPPGLDKRLLPETPGPCYSNSQPVWLCLTPRQPLEPHPPGE | |
| | GPSEWSSDTAEGRPCPYPHCQVLSAQPGSEEELEELCEQAV | |
| 7 | VSKGEELFTGVVPILVELDGDVNGHKFSVSGEGEGDATYGKLTLKFICTTG | GFP-NanoLuc |
| | KLPVPWPTLVTTLTYGVQCFSRYPDHMKQHDFFKSAMPEGYVQERTIFFK | reporter construct |
| | DDGNYKTRAEVKFEGDTLVNRIELKGIDFKEDGNILGHKLEYNYNSHNVYI | |
| | MADKQKNGIKVNFKIRHNIEDGSVQLADHYQQNTPIGDGPVLLPDNHYLS | |
| | TQSKLSKDPNEKRDHMVLLEFVTAAGITLGMDELYKGSGGSGGGGSAVG | |
| | QDTQEVIVVPHSLPFKVVVISAILALVVLTIISLIILIMLWQKKPRGGSGSVFT | |
| | LEDFVGDWRQTAGYNLDQVLEQGGVSSLFQNLGVSVTPIQRIVLSGENGL | |
| | KIDIHVIIPYEGLSGDQMGQIEKIFKVVYPVDDHHFKVILHYGTLVIDGVTP | |
| | NMIDYFGRPYEGIAVFDGKKITVTGTLWNGNKIIDERLINPDGSLLFRVTIN | |
| | GVTGWRLCERILA | |
| 8 | VSKGEELFTGVVPILVELDGDVNGHKFSVSGEGEGDATYGKLTLKFICTTG | GFP domain of SEQ |
| | KLPVPWPTLVTTLTYGVQCFSRYPDHMKQHDFFKSAMPEGYVQERTIFFK | ID NO: 7 |
| | DDGNYKTRAEVKFEGDTLVNRIELKGIDFKEDGNILGHKLEYNYNSHNVYI | |
| | MADKQKNGIKVNFKIRHNIEDGSVQLADHYQQNTPIGDGPVLLPDNHYLS | |
| | TQSKLSKDPNEKRDHMVLLEFVTAAGITLGMDELYK | |
| 9 | GSGGSGGGGSAVGQDTQEVIVVPHSLPFKVVVISAILALVVLTIISLIILIML | Transmembrane and |
| | WQKKPRGGSGS | linker domains of |
| | | SEQ ID NO: 7 |
| 10 | VFTLEDFVGDWRQTAGYNLDQVLEQGGVSSLFQNLGVSVTPIQRIVLSGE | Nanoluciferase |
| | NGLKIDIHVIIPYEGLSGDQMGQIEKIFKVVYPVDDHHFKVILHYGTLVIDG | domain of SEQ ID |
| | VTPNMIDYFGRPYEGIAVFDGKKITVTGTLWNGNKIIDERLINPDGSLLFRV | NO: 7 |
| | TINGVTGWRLCERILA | |
| 11 | DIQMTQSPSSLSASVGDRVTITCRASQSVSSAVAWYQQKPGKAPKLLIYSA | IgG light chain |
| | SSLYSGVSRFSGSRSGTDFTLTISSLQPEDFATYYCQQ | framework regions |
| | -[LC-CDR3]- | (constant domain |
| | TFGQGTKVEIKRTVAAPSVFIFPPSDSQLKSGTASVVCLLNNFYPREAKVQ | underlined) |
| | WKVDNALQSGNSQESVTEQDSKDSTYSLSSTLTLSKADYEKHKVYACEVT | |
| | HQGLSSPVTKSFNRGEC | |
| 356 | SDIQMTQSPSSLSASVGDRVTITCRASQSVSSAVAWYQQKPGKAPKLLIYS | IgG light chain |
| | ASSLYSGVSRFSGSRSGTDFTLTISSLQPEDFATYYCQQ | framework regions, |
| | -[LC-CDR3]- | alternative (constant |
| | TFGQGTKVEIKRTVAAPSVFIFPPSDSQLKSGTASVVCLLNNFYPREAKVQ | domain underlined) |
| | WKVDNALQSGNSQESVTEQDSKDSTYSLSSTLTLSKADYEKHKVYACEVT | |
| | HQGLSSPVTKSFNRGEC | |
| 12 | EISEVQLVESGGGLVQPGGSLRLSCAASGF | IgG heavy chain Fab |
| | -[HC-CDR1]- | framework regions |
| | WVRQAPGKGLEWV | (constant domain |
| | -[HC-CDR2]- | underlined) |
| | YADSVKGRFTISADTSKNTAYLQMNSLRAEDTAVYYCAR | |
| | -[HC-CDR3]- | |
| | DYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKP | |
| | SNTKVDKKVEPKSC | |
| 319 | DIQMTQSPSSLSASVGDRVTITCRASQSVGSALAWYQQKPGKAPKLLIYSA | Alternative IgG light |
| | SSLYSGVPSRFSGSRSGTDFTLTISSLQPEDFATYYCQQ | chain framework |
| | -[LCCDR3]- | regions (constant |
| | TFGQGTKVEIKRTVAAPSVFIFPPSDSQLKSGTASVVCLLNNFYPREAKVQ | domain underlined) |
| | WKVDNALQSGNSQESVTEQDSKDSTYSLSSTLTLSKADYEKHKVYACEVT | |
| | HQGLSSPVTKSFNRGEC | |
| 320 | EVQLVESGGGLVQPGGSLRLSCAASGEN | Alternative IgG heavy |
| | -[HCCDR1]- | chain Fab framework |
| | HWVRQAPGKGLEWVA | regions (constant |
| | -[HCCDR2]- | domain underlined) |
| | YADSVKGRFTISADTSKNTAYLQMNSLRAEDTAVYYCAR | |
| | -[HCCDR3]- | |
| | DYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKP | |
| | SNTKVDKKVEPKSC | |
| 321 | EVQLVESGGGLVQPGGSLRLSCAASGF | VH binder framework |
| | -[HCCDR1]- | regions |
| | IGWVRRAPGKGEELVA | |
| | -[HCCDR2]- | |
| | YADSVKGRFTISADTSKNTAYLQMNSLRAEDTAVYYCAR | |
| | -[HCCDR3]- | |
| | DYWGQGTLVTVSS | |
| 13 | DIQMTQSPSSLSASVGDRVTITCRASQSVSSAVAWYQQKPGKAPKLLIYSA | Bispecific IgG having |
| | SSLYSGVSRFSGSRSGTDFTLTISSLQPEDFATYYCQQ- | a single chain Fab as |
| | [LC-CDR3]- | one arm (constant |
| | TFGQGTKVEIKRTVAAPSVFIFPPSDSQLKSGTASVVCLLNNFYPREAKVQ | domains underlined) |
| | WKVDNALQSGNSQESVTEQDSKDSTYSLSSTLTLSKADYEKHKVYACEVT | |
| | HQGLSSPVTKSFNRGECGGSSGSGSGSTGTSSSGTGTSAGTTGTSASTSGSG | |
| | SGGGGGSGGGGSAGGTATAGASSGSEISEVQLVESGGGLVQPGGSLRLS- | |
| | CAASGF- | |
| | [HC-CDR1]- | |
| | WVRQAPGKGLEWV- | |
| | [HC-CDR2]- | |
| | YADSVKGRFTISADTSKNTAYLQMNSLRAEDTAVYYCAR- | |
| | [HC-CDR3]- | |
| | DYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKP | |
| | SNTKVDKKVEPKSC | |
| 357 | SDIQMTQSPSSLSASVGDRVTITCRASQSVSSAVAWYQQKPGKAPKLLIYS | Bispecific IgG having |
| | ASSLYSGVSRFSGSRSGTDFTLTISSLQPEDFATYYCQQ- | a single chain Fab as |
| | [LC-CDR3]- | one arm, alternative |
| | TFGQGTKVEIKRTVAAPSVFIFPPSDSQLKSGTASVVCLLNNFYPREAKVQ | (constant domains |
| | WKVDNALQSGNSQESVTEQDSKDSTYSLSSTLTLSKADYEKHKVYACEVT | underlined) |
| | HQGLSSPVTKSFNRGECGGSSGSGSGSTGTSSSGTGTSAGTTGTSASTSGSG | |
| | SGGGGGSGGGGSAGGTATAGASSGSEISEVQLVESGGGLVQPGGSLRLS- | |
| | CAASGF- | |
| | [HC-CDR1]- | |
| | WVRQAPGKGLEWV- | |
| | [HC-CDR2]- | |
| | YADSVKGRFTISADTSKNTAYLQMNSLRAEDTAVYYCAR- | |
| | [HC-CDR3]- | |
| | DYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKP | |
| | SNTKVDKKVEPKSC | |
| 14 | DKTHTCPPCPAPELLGGPSVFLFPPKPKDTLMISRTPEVTCVVVDVSHEDPE | IgG heavy chain Fc |
| | VKFNWYVDGVEVHNAKTKPREEQYNSTYRVVSVLTVLHQDWLNGKEYK | portion “knob” |
| | CKVSNKALPAPIEKTISKAKGQPREPQVYTLPPSRDELTKNQVSLWCLVKG | |
| | FYPSDIAVEWESNGQPENNYKTTPPVLDSDGSFFLYSKLTVDKSRWQQGN | |
| | VFSCSVMHEALHNHYTQKSLSLSPGKGGSHHHHHH | |
| 15 | DKTHTCPPCPAPELLGGPSVFLFPPKPKDTLMISRTPEVTCVVVDVSHEDPE | IgG heavy chain Fc |
| | VKFNWYVDGVEVHNAKTKPREEQYGSTYRVVSVLTVLHQDWLNGKEYK | portion “hole” |
| | CKVSNKALPAPIEKTISKAKGQPREPQVYTLPPSRDELTKNQVSLSCAVKGF | |
| | YPSDIAVEWESNGQPENNYKTTPPVLDSDGSFFLVSKLTVDKSRWQQGNV | |
| | FSCSVMHEALHNHYTQKSLSLSPGK | |
| 16 | EISEVQLVESGGGLVQPGGSLRLSCAASGF- | Fab-scFv construct - |
| | [ANTIGEN1-HC-CDR1]- | heavy chain-scFv |
| | WVRQAPGKGLEWV- | fusion (constant |
| | [ANTIGEN1-HC-CDR2]- | domain underlined) |
| | YADSVKGRFTISADTSKNTAYLQMNSLRAEDTAVYYCAR- | |
| | [ANTIGEN1-HC-CDR3]- | |
| | DYWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKP | |
| | SNTKVDKKVEPKSCDKTHTGGSGSAGGLNDIFEAQKIEWHESDIQMTQSPS | |
| | SLSASVGDRVTITCRASQSVSSAVAWYQQKPGKAPKLLIYSASSLYSGVSR | |
| | FSGSRSGTDFTLTISSLQPEDFATYYCQQ- | |
| | [ANTIGEN2-LC-CDR3]- | |
| | TFGQGTKVEIKGGGGSGGGGSGGGGSEISEVQLVESGGGLVQPGGSLRLSC | |
| | AASGF- | |
| | [ANTIGEN2-HC-CDR1]- | |
| | WVRQAPGKGLEWV- | |
| | [ANTIGEN2-HC-CDR2]- | |
| | YADSVKGRFTISADTSKNTAYLQMNSLRAEDTAVYYCAR- | |
| | [ANTIGEN2-HC-CDR3]- | |
| | DYWGQGTLVTVSS | |
| 17 | DIQMTQSPSSLSASVGDRVTITCRASQSVSSAVAWYQQKPGKAPKLLIYSA | Fab-scFv construct - |
| | SSLYSGVSRFSGSRSGTDFTLTISSLQPEDFATYYCQQ- | heavy chain-scFv |
| | [ANTIGEN1-LC-CDR3]- | fusion (light chain) |
| | TFGQGTKVEIKRTVAAPSVFIFPPSDSQLKSGTASVVCLLNNFYPREAKVQ | (constant domain |
| | WKVDNALQSGNSQESVTEQDSKDSTYSLSSTLTLSKADYEKHKVYACEVT | underlined) |
| | HQGLSSPVTKSFNRGEC | |
| 358 | SDIQMTQSPSSLSASVGDRVTITCRASQSVSSAVAWYQQKPGKAPKLLIYS | Fab-scFv construct - |
| | ASSLYSGVSRFSGSRSGTDFTLTISSLQPEDFATYYCQQ- | heavy chain-scFv |
| | [ANTIGEN1-LC-CDR3]- | fusion, alternative |
| | TFGQGTKVEIKRTVAAPSVFIFPPSDSQLKSGTASVVCLLNNFYPREAKVQ | (light chain) (constant |
| | WKVDNALQSGNSQESVTEQDSKDSTYSLSSTLTLSKADYEKHKVYACEVT | domain underlined) |
| | HQGLSSPVTKSFNRGEC | |
| 18 | YYDSSYALF | RNF43 LC-CDR3 |
| 19 | SGWPF | RNF43 LC-CDR3 |
| 20 | GYSDLI | RNF43 LC-CDR3 |
| 21 | VYPPI | RNF43 LC-CDR3 |
| 22 | AYPI | RNF43 LC-CDR3 |
| 23 | SKYSNQLI | RNF43 LC-CDR3 |
| 24 | WSWPYPL | RNF43 LC-CDR3 |
| 25 | SSFIWPL | RNF43 LC-CDR3 |
| 26 | SHWEKLI | RNF43 LC-CDR3 |
| 27 | GRSWPV | RNF43 LC-CDR3 |
| 28 | KVRWPLI | RNF43 LC-CDR3 |
| 29 | SSKGLI | RNF43 LC-CDR3 |
| 30 | ALYYPI | RNF43 LC-CDR3 |
| 31 | SYYWPV | RNF43 LC-CDR3 |
| 32 | YVYSYPF | RNF43 LC-CDR3 |
| 33 | WWYFPI | RNF43 LC-CDR3 |
| 34 | SSSPF | ZNRF3 LC-CDR3 |
| 35 | SYYPI | ZNRF3 LC-CDR3 |
| 36 | SSYYFWSPI | ZNRF3 LC-CDR3 |
| 37 | YAYYSPF | ZNRF3 LC-CDR3 |
| 38 | SYYSLF | ZNRF3 LC-CDR3 |
| 39 | GWVVPI | ZNRF3 LC-CDR3 |
| 40 | SWDSLI | ZNRF3 LC-CDR3 |
| 41 | SRRYPV | ZNRF3 LC-CDR3 |
| 42 | YSGSLI | ZNRF3 LC-CDR3 |
| 43 | SPYELI | ZNRF3 LC-CDR3 |
| 44 | SSSDPI | ZNRF3 LC-CDR3 |
| 45 | SRRYPV | ZNRF3 LC-CDR3 |
| 46 | GYKGSSLI | ZNRF3 LC-CDR3 |
| 47 | SWGWPI | ZNRF3 LC-CDR3 |
| 48 | PYPGMQPI | ZNRF3 LC-CDR3 |
| 49 | MSSSPI | ZNRF3 LC-CDR3 |
| 50 | SSSSLI | ZNRF3 LC-CDR3 |
| 51 | SSHYLI | ZNRF3 LC-CDR3 |
| 52 | YSFSSLI | ZNRF3 LC-CDR3 |
| 53 | SRRYPV | ZNRF3 LC-CDR3 |
| 54 | TWSVVPI | ZNRF3 LC-CDR3 |
| 55 | SRRYPV | ZNRF3 LC-CDR3 |
| 56 | SHPAFPF | GRAIL LC-CDR3 |
| 57 | GGGWYPF | GRAIL LC-CDR3 |
| 58 | SHAYPI | GRAIL LC-CDR3 |
| 59 | YYYLI | GRAIL LC-CDR3 |
| 60 | HHSLI | GRAIL LC-CDR3 |
| 61 | ISYYSI | RNF43 HC-CDR1 |
| 62 | IYSYYM | RNF43 HC-CDR1 |
| 63 | IYYYSI | RNF43 HC-CDR1 |
| 64 | LSYSYI | RNF43 HC-CDR1 |
| 65 | IYYYSM | RNF43 HC-CDR1 |
| 66 | VSYYYI | RNF43 HC-CDR1 |
| 67 | FYSYSI | RNF43 HC-CDR1 |
| 68 | FYSYSI | RNF43 HC-CDR1 |
| 69 | IYSYYI | RNF43 HC-CDR1 |
| 70 | FYSYSI | RNF43 HC-CDR1 |
| 71 | IYSSSI | RNF43 HC-CDR1 |
| 72 | VSSSSI | RNF43 HC-CDR1 |
| 73 | VYSSSI | RNF43 HC-CDR1 |
| 74 | VYYSSI | RNF43 HC-CDR1 |
| 75 | IYYSYI | RNF43 HC-CDR1 |
| 76 | VSYSSI | RNF43 HC-CDR1 |
| 77 | ISYSSI | ZRNF3 HC-CDR1 |
| 78 | LYYSYI | ZRNF3 HC-CDR1 |
| 79 | LYYSSI | ZRNF3 HC-CDR1 |
| 80 | ISYYSM | ZRNF3 HC-CDR1 |
| 81 | LSSYSI | ZRNF3 HC-CDR1 |
| 82 | ISYYYM | ZRNF3 HC-CDR1 |
| 83 | ISSSSM | ZRNF3 HC-CDR1 |
| 84 | ISYSSI | ZRNF3 HC-CDR1 |
| 85 | VYSYYI | ZRNF3 HC-CDR1 |
| 86 | VYSSYI | ZRNF3 HC-CDR1 |
| 87 | VYSSSI | ZRNF3 HC-CDR1 |
| 88 | VSYSSI | ZRNF3 HC-CDR1 |
| 89 | VYYSSI | ZRNF3 HC-CDR1 |
| 90 | FSSYSI | ZRNF3 HC-CDR1 |
| 91 | FYYYSI | ZRNF3 HC-CDR1 |
| 92 | ISSSYI | ZRNF3 HC-CDR1 |
| 93 | FYSSYI | ZRNF3 HC-CDR1 |
| 94 | FYSYSI | ZRNF3 HC-CDR1 |
| 95 | VSSSSI | ZRNF3 HC-CDR1 |
| 96 | ISYSSI | ZRNF3 HC-CDR1 |
| 97 | VYSYYI | ZRNF3 HC-CDR1 |
| 98 | ISYSSI | ZRNF3 HC-CDR1 |
| 99 | NLYSYSIH | GRAIL HC-CDR1 |
| 100 | NIYYSSMH | GRAIL HC-CDR1 |
| 101 | NLYSYYMH | GRAIL HC-CDR1 |
| 102 | NIYYYSMH | GRAIL HC-CDR1 |
| 103 | NISYSSMH | GRAIL HC-CDR1 |
| 104 | RTVAAPSVFIFPPSDEQLKSGTASVVCLLNNFYPREAKVQWKVDNALQSG | Constant domain CL1 |
| | NSQESVTEQDSKDSTYSLSSTLTLSKADYEKHKVYACEVTHQGLSSPVTKS | version 1 |
| | FNRGEC | |
| 334 | RTVAAPSVFIFPPSDSQLKSGTASVVCLLNNFYPREAKVQWKVDNALQSG | Constant domain CL1 |
| | NSQESVTEQDSKDSTYSLSSTLTLSKADYEKHKVYACEVTHQGLSSPVTKS | version 2 |
| | FNRGEC | |
| 105 | ASTKGPSVFPLAPSSKSTSGGTAALGCLVKDYFPEPVTVSWNSGALTSGVH | Constant domain CH1 |
| | TFPAVLQSSGLYSLSSVVTVPSSSLGTQTYICNVNHKPSNTKVDKKV | |
| 106 | DIQMTQSPSSLSASVGDRVTITCRASQDVSTAVAWYQQKPGKAPKLLIYSA | LC variable domain, |
| | SFLYSGVPSRFSGSGSGTDFTLTISSLQPEDFATYYCQQYLYHPATFGQGTK | anti-PD-L1 |
| | VEIK | |
| 107 | EVQLVESGGGLVQPGGSLRLSCAASGFTFSDSWIHWVRQAPGKGLEWVA | HC variable domain, |
| | WISPYGGSTYYADSVKGRFTISADTSKNTAYLQMNSLRAEDTAVYYCARR | anti-PD-L1 |
| | HWPGGFDYWGQGTLVTVSS | |
| 108 | DIQMTQSPSSLSASVGDRVTITCRASQDVNTAVAWYQQKPGKAPKLLIYSA | LC variable domain, |
| | SFLYSGVPSRFSGSRSGTDFTLTISSLQPEDFATYYCQQHYTTPPTFGQGTKL | anti-HER2 |
| | EIK | |
| 109 | EVQLVESGGGLVQPGGSLRLSCAASGFNIKDTYIHWVRQAPGKGLEWVAR | HC variable domain, |
| | IYPTNGYTRYADSVKGRFTISADTSKNTAYLQMNSLRAEDTAVYYCSRWG | anti-HER2 |
| | GDGFYAMDYWGQGTLVTVSS | |
| 110 | DILLTQSPVILSVSPGERVSFSCRASQSIGTNIHWYQQRTNGSPRLLIKYASE | LC variable domain, |
| | SISGIPSRFSGSGSGTDFTLSINSVESEDIADYYCQQNNNWPTTFGAGTKLEL | anti-EGFR |
| | K | |
| 111 | QVQLKQSGPGLVQPSQSLSITCTVSGFSLTNYGVHWVRQSPGKGLEWLGVI | HC variable domain, |
| | WSGGNTDYNTPFTSRLSINKDNSKSQVFFKMNSLQSNDTAIYYCARALTY | anti-EGFR |
| | YDYEFAYWGQGTLVTVSA | |
| 112 | EIVLTQSPGTLSLSPGERATLSCRASQSVGSSYLAWYQQKPGQAPRLLIYGA | LC variable domain, |
| | FSRATGIPDRFSGSGSGTDFTLTISRLEPEDFAVYYCQQYGSSPWTFGQGTK | anti-CTLA4 |
| | VEIK | |
| 113 | QVQLVESGGGVVQPGRSLRLSCAASGFTFSSYTMHWVRQAPGKGLEWVT | HC variable domain, |
| | FISYDGNNKYYADSVKGRFTISRDNSKNTLYLQMNSLRAEDTAIYYCART | anti-CTLA4 |
| | GWLGPFDYWGQGTLVTVSS | |
| 114 | DIQMTQSPSSLSASVGDRVTITCRASQSVSSAVAWYQQKPGKAPKLLIYSA | LC variable domain, |
| | SSLYSGVPSRFSGSRSGTDFTLTISSLQPEDFATYYCQQSYYYYPITFGQGTK | anti-CDCP1 |
| | VEIK | |
| 115 | EISEVQLVESGGGLVQPGGSLRLSCAASGFNLSYYYIHWVRQAPGKGLEW | HC variable domain, |
| | VASIYSSSSYTSYADSVKGRFTISADTSKNTAYLQMNSLRAEDTAVYYCAR | anti-CDCP1 |
| | AYYGFDYWGQGTLVTVSS | |
| 359 | AASDIQMTQSPSSLSASVGDRVTITCRASQSVSSAVAWYQQKPGKAPKLLI | LC variable domain, |
| | YSASSLYSGVPSRFSGSRSGTDFTLTISSLQPEDFATYYCQQSYYYYPITFGQ | anti-CDCP1; |
| | GTKVEIK | alternative |
| 360 | AAQPEISEVQLVESGGGLVQPGGSLRLSCAASGFNLSYYYIHWVRQAPGK | HC variable domain, |
| | GLEWVASIYSSSSYTSYADSVKGRFTISADTSKNTAYLQMNSLRAEDTAVY | anti-CDCP1; |
| | YCARAYYGFDYWGQGTLVTVSS | alternative |
| 116 | WSYYGLI | CDR, 1° Ab LC3 |
| 117 | LSSYYIH | CDR, 1º Ab HC1 |
| 118 | SIYSSSSYTS | CDR, 1º Ab HC2 |
| 119 | SHGPVYSGYWYYYWYWGFD | CDR, 1° Ab HC3 |
| 120 | SIYPYYGYTY | RNF43 HC-CDR2 |
| 121 | YISPYYSYTY | RNF43 HC-CDR2 |
| 122 | SIYSSSGYTS | RNF43 HC-CDR2 |
| 123 | SISPSYGYTY | RNF43 HC-CDR2 |
| 124 | SISPYYSYTS | RNF43 HC-CDR2 |
| 125 | SISSYYGSTS | RNF43 HC-CDR2 |
| 126 | SISSSSGSTS | RNF43 HC-CDR2 |
| 127 | SISPYYGSTS | RNF43 and ZNRF3 |
| | | HC-CDR2 |
| 128 | SIYSYYGSTY | RNF43 HC-CDR2 |
| 129 | SISSYYGSTS | RNF43 and ZNRF3 |
| | | HC-CDR2 |
| 130 | SISSYYGYTS | RNF43 and ZNRF3 |
| | | HC-CDR2 |
| 131 | SISSYSGYTY | RNF43 HC-CDR2 |
| 132 | YISSYSGSTY | RNF43 HC-CDR2 |
| 133 | SISSYYSYTS | RNF43 and ZNRF3 |
| | | HC-CDR2 |
| 134 | SISSYSGYTS | RNF43 HC-CDR2 |
| 135 | YISSYYGSTS | ZNRF3 HC-CDR2 |
| 136 | SIYPSYGSTY | ZNRF3 and PD-L1 |
| | | HC-CDR2 |
| 137 | SISPSYSYTS | ZNRF3 HC-CDR2 |
| 138 | SISPYYGYTS | ZNRF3 and PD-L1 |
| | | HC-CDR2 |
| 139 | SIYPYYSSTY | ZNRF3 and GRAIL |
| | | HC-CDR2 |
| 140 | YIYPYYGSTS | ZNRF3 HC-CDR2 |
| 141 | SIYSYYSSTS | ZNRF3 HC-CDR2 |
| 142 | SISPSYGSTY | ZNRF3 HC-CDR2 |
| 143 | SISSYYSSTS | ZNRF3 HC-CDR2 |
| 144 | SISPYYGSTY | ZNRF3 HC-CDR2 |
| 145 | SIYSYYGSTS | ZNRF3 HC-CDR2 |
| 146 | SIYSYYGYTS | ZNRF3 HC-CDR2 |
| 147 | SISPYSGYTS | ZNRF3 HC-CDR2 |
| 148 | SISSSYGYTY | GRAIL HC-CDR2 |
| 149 | SIYPYYGSTY | GRAIL and PD-L1 |
| | | HC-CDR2 |
| 150 | SISPYSRYTS | GRAIL HC-CDR2 |
| 151 | SIYPYYSYTY | GRAIL HC-CDR2 |
| 152 | GSYFYGM | RNF43 HC-CDR3 |
| 153 | AYADSWPGYSWGSSDFAL | RNF43 HC-CDR3 |
| 154 | YPYWYFDGF | RNF43 HC-CDR3 |
| 155 | PYHPFGGHYWWPYYYHGL | RNF43 HC-CDR3 |
| 156 | YGYYGWDYHRYSAF | RNF43 HC-CDR3 |
| 157 | EYYFGL | RNF43 HC-CDR3 |
| 158 | WSWYNHGSSSWAM | RNF43 HC-CDR3 |
| 159 | WSYWYSSYYGAM | RNF43 HC-CDR3 |
| 160 | IFAMGL | RNF43 HC-CDR3 |
| 161 | NGYNWGM | RNF43 HC-CDR3 |
| 162 | SYWQSYMAM | RNF43 HC-CDR3 |
| 163 | DIQMDSGYKWHPWLGM | RNF43 HC-CDR3 |
| 164 | SPYGHWYGYYGRQGGL | RNF43 HC-CDR3 |
| 165 | YYFYHSYGSYAL | RNF43 HC-CDR3 |
| 166 | EWYVGM | RNF43 HC-CDR3 |
| 167 | SYSYTGM | RNF43 HC-CDR3 |
| 168 | GWYPYSYSRDAM | ZNRF3 HC-CDR3 |
| 169 | GYAI | A18 HC-CDR3 |
| 170 | SWVYSWGM | A19 HC-CDR3 |
| 171 | WVGYYPPYYFSGSYGM | A20 HC-CDR3 |
| 172 | RYSYSYWGFHPAF | A21 HC-CDR3 |
| 173 | DVDWPYYFYAI | A22 HC-CDR3 |
| 174 | GAYGAPFYYYYFWWDRGM | A23 HC-CDR3 |
| 175 | NSSYPYSWGSKYSWLAL | ZNRF3 HC-CDR3 |
| 176 | SGWGWLYYWYPHGI | ZNRF3 HC-CDR3 |
| 177 | SSIYYAM | ZNRF3 HC-CDR3 |
| 178 | SIQLAKWGYYWIGSSGM | ZNRF3 HC-CDR3 |
| 179 | YKVYHWPVQWQRYWPAM | ZNRF3 HC-CDR3 |
| 180 | QSMSYWSRQYGF | ZNRF3 HC-CDR3 |
| 181 | DWYYVSGYYFSAF | ZNRF3 HC-CDR3 |
| 182 | QPWMYWWLKYAI | ZNRF3 HC-CDR3 |
| 183 | SWWEYFYPYGWYQYAI | ZNRF3 HC-CDR3 |
| 184 | KPWYSERFYQGIHYTAM | ZNRF3 HC-CDR3 |
| 185 | SWYPQYDWRYYAL | ZNRF3 HC-CDR3 |
| 186 | EEWYSSGMWWYSYGGI | ZNRF3 HC-CDR3 |
| 187 | YYWGYKGHYPAI | ZNRF3 HC-CDR3 |
| 188 | TVRGSKKPYFSGWAM | GRAIL HC-CDR3 |
| 189 | HHSYFFGGL | GRAIL HC-CDR3 |
| 190 | DPYFSHVFSYWGF | GRAIL HC-CDR3 |
| 191 | DFFSSYYPVVAASAGI | GRAIL HC-CDR3 |
| 192 | DSPYSYYSPWGGM | GRAIL HC-CDR3 |
| 193 | YYHPI | PD-L1 LC-CDR3 |
| 194 | SYYPF | PD-L1 LC-CDR3 |
| 195 | AYYSPI | PD-L1 LC-CDR3 |
| 196 | HYTTPP | PD-L1 LC-CDR3 |
| 197 | AWGPF | PD-L1 LC-CDR3 |
| 198 | SSPYLF | PD-L1 LC-CDR3 |
| 199 | YRYADALI | PD-L1 LC-CDR3 |
| 200 | SRYVSPI | PD-L1 LC-CDR3 |
| 201 | YGSYPI | PD-L1 LC-CDR3 |
| 202 | DYYPYWHAPF | PD-L1 LC-CDR3 |
| 203 | PYSLI | PD-L1 LC-CDR3 |
| 204 | YWWPGSLI | PD-L1 LC-CDR3 |
| 205 | SSWEPV | PD-L1 LC-CDR3 |
| 206 | SYSYLV | PD-L1 LC-CDR3 |
| 207 | SMYYLI | PD-L1 LC-CDR3 |
| 208 | SWPGYPI | PD-L1 LC-CDR3 |
| 209 | DYFGLI | PD-L1 LC-CDR3 |
| 210 | SSWWSPI | PD-L1 LC-CDR3 |
| 211 | SWPGSPV | PD-L1 LC-CDR3 |
| 212 | GGYWLV | PD-L1 LC-CDR3 |
| 213 | HYQPLI | PD-L1 LC-CDR3 |
| 214 | PIWYEPI | PD-L1 LC-CDR3 |
| 215 | SWPQYPV | PD-L1 LC-CDR3 |
| 216 | WNYALI | PD-L1 LC-CDR3 |
| 217 | YNWSQLI | PD-L1 LC-CDR3 |
| 218 | YHWPSELF | PD-L1 LC-CDR3 |
| 219 | SGQYWPF | PD-L1 LC-CDR3 |
| 220 | SASWEPV | PD-L1 LC-CDR3 |
| 221 | SYYYLV | PD-L1 LC-CDR3 |
| 222 | SYPHSLI | PD-L1 LC-CDR3 |
| 223 | SSYSLF | PD-L1 LC-CDR3 |
| 224 | YNLSLV | PD-L1 LC-CDR3 |
| 225 | LSYYSI | PD-L1 HC-CDR1 |
| 226 | LYSYYI | PD-L1 HC-CDR1 |
| 227 | LSYSYM | PD-L1 HC-CDR1 |
| 228 | IKDTYI | PD-L1 HC-CDR1 |
| 229 | LSSSYM | PD-L1 HC-CDR1 |
| 230 | ISSYYI | PD-L1 HC-CDR1 |
| 231 | ISSYSI | PD-L1 HC-CDR1 |
| 232 | LSSSYI | PD-L1 HC-CDR1 |
| 233 | ISSYSM | PD-L1 HC-CDR1 |
| 234 | VSSYSI | PD-L1 HC-CDR1 |
| 235 | IYYYYI | PD-L1 HC-CDR1 |
| 236 | FSSSSI | PD-L1 HC-CDR1 |
| 237 | FSYSSI | PD-L1 HC-CDR1 |
| 238 | LSYSSI | PD-L1 HC-CDR1 |
| 239 | ISSSSI | PD-L1 HC-CDR1 |
| 240 | LSSSSI | PD-L1 HC-CDR1 |
| 241 | FSYSYI | PD-L1 HC-CDR1 |
| 242 | IYYSSI | PD-L1 HC-CDR1 |
| 243 | SISPSYGYTS | PD-L1 HC-CDR2 |
| 244 | SISPSSSYTY | PD-L1 HC-CDR2 |
| 245 | RIYPTNGYTR | PD-L1 HC-CDR2 |
| 246 | SIYSYSGYTS | PD-L1 HC-CDR2 |
| 247 | SIYPYSSYTS | PD-L1 HC-CDR2 |
| 248 | SISSSSSYTS | PD-L1 HC-CDR2 |
| 249 | SIYPSSSYTS | PD-L1 HC-CDR2 |
| 250 | SISSSYGYTS | PD-L1 HC-CDR2 |
| 251 | YISPYSSYTS | PD-L1 HC-CDR2 |
| 252 | SIYSYSGYTY | PD-L1 HC-CDR2 |
| 253 | SIYSSSGSTS | PD-L1 HC-CDR2 |
| 254 | YISSSSGSTS | PD-L1 HC-CDR2 |
| 255 | SIYPYSGYTS | PD-L1 HC-CDR2 |
| 256 | YIYSYSGSTY | PD-L1 HC-CDR2 |
| 257 | SISSYYGSTY | PD-L1 HC-CDR2 |
| 258 | YISSYSGSTS | PD-L1 HC-CDR2 |
| 259 | SIYSSYGSTS | PD-L1 HC-CDR2 |
| 260 | SIYSSYGYTS | PD-L1 HC-CDR2 |
| 261 | SISPSSSYTS | PD-L1 HC-CDR2 |
| 262 | SISSYYGYTY | PD-L1 HC-CDR2 |
| 263 | SISSYSGSTS | PD-L1 HC-CDR2 |
| 264 | SPWDPWAHHGHGI | PD-L1 HC-CDR3 |
| 265 | GVASYYYSASYSWYGGM | PD-L1 HC-CDR3 |
| 266 | YYYWHYFWDAF | PD-L1 HC-CDR3 |
| 267 | SPYAPGYFAVHGAPVGGM | PD-L1 HC-CDR3 |
| 268 | GSYRFWDAF | PD-L1 HC-CDR3 |
| 269 | HSYSSGF | PD-L1 HC-CDR3 |
| 270 | GYFWSYSGF | PD-L1 HC-CDR3 |
| 271 | SDHGVAYGI | PD-L1 HC-CDR3 |
| 272 | GASSDWYFWSSGL | PD-L1 HC-CDR3 |
| 273 | GDWWGAL | PD-L1 HC-CDR3 |
| 274 | YQHLAL | PD-L1 HC-CDR3 |
| 275 | SPSIVWAWHWQYGPGF | PD-L1 HC-CDR3 |
| 276 | SFMYGTWYPYGF | PD-L1 HC-CDR3 |
| 277 | YWWAFHWESHSYQPSYGF | PD-L1 HC-CDR3 |
| 278 | SDFMLHWHWFGM | PD-L1 HC-CDR3 |
| 279 | SHSYGSWYAYGL | PD-L1 HC-CDR3 |
| 280 | WEESRYWYKYYYQGGL | PD-L1 HC-CDR3 |
| 281 | NWSGYLAM | PD-L1 HC-CDR3 |
| 282 | SQSYGSWYAYGL | PD-L1 HC-CDR3 |
| 283 | TSYWEYWYWFGL | PD-L1 HC-CDR3 |
| 284 | HYGFAM | PD-L1 HC-CDR3 |
| 285 | GYYSSYSSWYLYGSDSAI | PD-L1 HC-CDR3 |
| 286 | GYFPAM | PD-L1 HC-CDR3 |
| 287 | HRYFAM | PD-L1 HC-CDR3 |
| 288 | YGGYGEYFSWYPYGM | PD-L1 HC-CDR3 |
| 289 | PYWWSGQGYWGF | PD-L1 HC-CDR3 |
| 290 | GEGQQYKWSPYGL | PD-L1 HC-CDR3 |
| 291 | WYYAWHMGVKGYQGF | PD-L1 HC-CDR3 |
| 292 | QEWYYGFGAYKYHWATGL | PD-L1 HC-CDR3 |
| 293 | WRSLAL | PD-L1 HC-CDR3 |
| 294 | ESYWWWSYWHLGL | PD-L1 HC-CDR3 |
| 295 | GDDHVYWWWFGM | PD-L1 HC-CDR3 |
| 296 | DYSAYYAM | PD-L1 HC-CDR3 |
| 297 | QGWLAL | PD-L1 HC-CDR3 |
| 298 | SYGEGSYTFWIWAGYGAL | PD-L1 HC-CDR3 |
| 299 | SYNYHYYTPYGF | PD-L1 HC-CDR3 |
| 300 | YPYEVSWTPYGM | PD-L1 HC-CDR3 |
| 301 | AEWYLHFEQGFGF | PD-L1 HC-CDR3 |
| 302 | YYYKYMAM | PD-L1 HC-CDR3 |
| 303 | YWWPFHWESHSYQPSYGF | PD-L1 HC-CDR3 |
| 304 | SYNYQGDNWHEYYPSGL | PD-L1 HC-CDR3 |
| 305 | YYGYGM | PD-L1 HC-CDR3 |
| 306 | AFSYYDY | RNF43 HC-CDR1 |
| 307 | DFYSSDD | RNF43 HC-CDR1 |
| 308 | AIYSYYYD | RNF43 HC-CDR1 |
| 309 | AIYYSYD | RNF43 HC-CDR1 |
| 310 | RISSYYYD | RNF43 HC-CDR1 |
| 311 | RIYPYSSYTS | RNF43 HC-CDR2 |
| 312 | RIYSSYGSTY | RNF43 HC-CDR2 |
| 313 | RIYPYSGYTS | RNF43 HC-CDR2 |
| 314 | YSPGYYPFRGWGGM | RNF43 HC-CDR3 |
| 315 | YAWRPSGGYYSYAM | RNF43 HC-CDR3 |
| 316 | GSYYFGYAF | RNF43 HC-CDR3 |
| 317 | WGWSYDPAGYAYAM | RNF43 HC-CDR3 |
| 318 | WYDSPGF | RNF43 HC-CDR3 |
| 322 | DIQMTQSPSSLSASVGDRVTITCRASQSVGSALAWYQQKPGKAPKLLIYSA | RNF43 A4 Light |
| | SSLYSGVPSRFSGSRSGTDFTLTISSLQPEDFATYYCQQ [symbol] TFGQGTKVE | Chain Construct (LC |
| | IKRTVAAPSVFIFPPSDSQLKSGTASVVCLLNNFYPREAKVOWKVDNALQS | CDR3 bolded) |
| | GNSQESVTEQDSKDSTYSLSSTLTLSKADYEKHKVYACEVTHQGLSSPVTK | (constant domain |
| | SFNRGEC | underlined) |
| 323 | EISEVQLVESGGGLVQPGGSLRLSCAASGFN [symbol] HWVRQAPGKGLEWV | RNF43 A4 Heavy |
| | A [symbol] YADSVKGRFTISADTSKNTAYLQMNSLRAEDTAVYYCAR [symbol] | Chain Construct |
| | [symbol] [symbol] DYWGQGTLVTVSSASTKGPSVFPLAPSSKSTSG | (CDRs bolded) |
| | GTAALGCLVKDYFPEPVTVSWNSGALTSGVHTFPAVLQSSGLYSLSSVVT | (constant domain |
| | VPSSSLGTQTYICNVNHKPSNTKVDKKVEPKSC | underlined) |
| 324 | DIQMTQSPSSLSASVGDRVTITCRASQSVGSALAWYQQKPGKAPKLLIYSA | RNF43 A6 Light |
| | SSLYSGVPSRFSGSRSGTDFTLTISSLQPEDFATYYCQQ [symbol] TFGQGT | Chain Construct (LC |
| | KVEIKRTVAAPSVFIFPPSDSQLKSGTASVVCLLNNFYPREAKVQWKVDNA | CDR3 bolded) |
| | LQSGNSQESVTEQDSKDSTYSLSSTLTLSKADYEKHKVYACEVTHQGLSSP | (constant domain |
| | VTKSFNRGEC | underlined) |
| 325 | EISEVQLVESGGGLVQPGGSLRLSCAASGFN [symbol] HWVRQAPGKGLEW | RNF43 A6 Heavy |
| | VA [symbol] YADSVKGRFTISADTSKNTAYLQMNSLRAEDTAVYYCAR | Chain Construct |
| | [symbol] DYWGQGTLVTVSSASTKGPSVFPLAPSSKSTSGGTAALGCLVKD | (CDRs bolded) |
| | YFPEPVTVSWNSGALTSGVHTFPAVLQSSGLYSLSSVVTVPSSSLGTQTYIC | (constant domain |
| | NVNHKPSNTKVDKKVEPKSC | underlined) |
| 326 | DIQLTQMPSSLSASVGDRVTITCRASQSVGSALAWYQQKPGKAPKLLIYSA | anti-RNF43 Fab LC in |
| | SSLYSGVPSRFSGSRSGTDFTLTISSLQPEDFATYYCQQAYPITFGQGTKVEI | the conjugate |
| | KRTVAAPSVFIFPPSDSQLKSGTASVVCLLNNFYPREAKVQWKVDNALQS | |
| | GNSQESVTEQDSKDSTYSLSSTLTLSKADYEKHKVYACEVTHQGLSSPVTK | |
| | SFNRGEC | |
| 327 | EISEVQLVESGGGLVQPGGSLRLSCAASGFNIYYYSLHWVRQAPGKGLEW | anti-RNF43 Fab HC |
| | VASISPYYSYTSYADSVKGRFTISADTSKNTAYLQLNSLRAEDTAVYYCAR | in the conjugate |
| | YGYYGWDYHRYSAFDYWGQGTLVTVSSASTKGPSVFPLAPSSKSTSGGTA | |
| | ALGCLVKDYFPEPVTVSWNSGALTSGVHTFPAVLQSSGLYSLSSVVTVPSS | |
| | SLGTQTYICNVNHKPSNTKVDKKVEPKSCDKTHTGGSGSAGGLNDIFEAQ | |
| | KIEWHE | |
| 328 | EVQLLESGGGLVQPGGSLRLSCAASGFTFSLYSMNWVRQAPGKGLEWVSS | Anti-MMP14 Fab |
| | IYSSGGSTLYADSVKGRFTISRDNSKNTLYLQMNSLRAEDTAVYYCARGR | HC-binder 1 (CDRs |
| | AFDIWGQGTMVTVSSASTKGPSVFPLAPSSKSTSGGTAALGCLVKDYFPEP | bolded) |
| | VTVSWNSGALTSGVHTFPAVLQSSGLYSLSSVVTVPSSSLGTQTYICNVNH | |
| | KPSNTKVDKKV | |
| 329 | DIQMTQSPSSLSASVGDRVTITCRASQSVGTYLNWYQQKPGKAPKLLIYA | Anti-MMP14 Fab LC- |
| | TSNLRSGVPSRFSGSGSGTDFTLTISSLQPEDFATYYCQQSYSIPRFTFGPG | binder 1 (CDRs |
| | TKVDIKRTVAAPSVFIFPPSDEQLKSGTASVVCLLNNFYPREAKVQWKVDN | bolded) |
| | ALQSGNSQESVTEQDSKDSTYSLSSTLTLSKADYEKHKVYACEVTHQGLSS | |
| | PVTKSFNRGEC | |
| 330 | EVQLVESGGGLVQPGGSLRLSCAASGFNLSSSSMHWVRQAPGKGLEWVA | Anti-MMP14 Fab |
| | SIYPSYSYTYYADSVKGRFTISADTSKNTAYLQMNSLRAEDTAVYYCARV | HC-binder 2 (CDRs |
| | KLQKDKSHQWIRNLVATPYGRYVMDYWGQGTLVTVSSASTKGPSVFPL | bolded) |
| | APSSKSTSGGTAALGCLVKDYFPEPVTVSWNSGALTSGVHTFPAVLQSSGL | |
| | YSLSSVVTVPSSSLGTQTYICNVNHKPSNTKVDKKV | |
| 331 | DIQMTQSPSSLSASVGDRVTITCRASQSVSSAVAWYQQKPGKAPKLLIYSA | Anti-MMP14 Fab LC- |
| | SSLYSGVPSRFSGSRSGTDFTLTISSLQPEDFATYYCQQYGYPITFGQGTKV | binder 2 (CDRs |
| | EIKRTVAAPSVFIFPPSDEQLKSGTASVVCLLNNFYPREAKVQWKVDNALQ | bolded) |
| | SGNSQESVTEQDSKDSTYSLSSTLTLSKADYEKHKVYACEVTHQGLSSPVT | |
| | KSFNRGEC | |
| 332 | DIQMTQSPSSLSASVGDRVTITCRASQSVGSALAWYQQKPGKAPKLLIYS | RNF43 A5 Light |
| | ASSLYSGVPSRFSGSRSGTDFTLTISSLQPEDFATYYCQQ [symbol] TFGQGTK | Chain Construct |
| | VEIKRTVAAPSVFIFPPSDSQLKSGTASVVCLLNNFYPREAKVQWKVDNA | (LC CDR3 bolded) |
| | LQSGNSQESVTEQDSKDSTYSLSSTLTLSKADYEKHKVYACEVTHQGLSS | (constant domain |
| | PVTKSFNRGEC | underlined) |
| 333 | EISEVQLVESGGGLVQPGGSLRLSCAASGFN [symbol] HWVRQAPGKGLE | RNF43 A5 Heavy |
| | WVA [symbol] YADSVKGRFTISADTSKNTAYLQMNSLRAEDTAVYY | Chain Construct |
| | CAR [symbol] [symbol] DYWGQGTLVTVSSASTKGPSVFPLAPSSKSTS | (CDRs bolded) |
| | GGTAALGCLVKDYFPEPVTVSWNSGALTSGVHTFPAVLQSSGLYSLSSV | (constant domain |
| | VTVPSSSLGTQTYICNVNHKPSNTKVDKKVEPKSC | underlined) |
| 335 | EPKSCDKTHTCPPCPAPELLGGPSVFLFPPKPKDTLMISRTPEVTCVVVDV | CH2-CH3 (Knob |
| | SHEDPEVKFNWYVDGVEVHNAKTKPREEQYGSTYRVVSVLTVLHQDW | construct with |
| | LNGKEYKCKVSNKALPAPIEKTISKAKGQPREPQVYTLPPSRDELTKNQV | N297G) |
| | SLWCLVKGFYPSDIAVEWESNGQPENNYKTTPPVLDSDGSFFLYSKLTV | |
| | DKSRWQQGNVFSCSVMHEALHNHYTQKSLSLSPGK | |
| 336 | EPKSCDKTHTCPPCPAPELLGGPSVFLFPPKPKDTLMISRTPEVTCVVVDV | CH2-CH3 (Hole |
| | SHEDPEVKFNWYVDGVEVHNAKTKPREEQYGSTYRVVSVLTVLHQDW | construct with |
| | LNGKEYKCKVSNKALPAPIEKTISKAKGQPREPQVYTLPPSRDELTKNQV | N297G) |
| | SLSCAVKGFYPSDIAVEWESNGQPENNYKTTPPVLDSDGSFFLVSKLTVD | |
| | KSRWQQGNVFSCSVMHEALHNHYTQKSLSLSPGK | |
| 337 | EPKSCDKTHTCPPCPAPELLGGPSVFLFPPKPKDTLMISRTPEVTCVVVDV | CH2-CH3 (WT |
| | SHEDPEVKFNWYVDGVEVHNAKTKPREEQYNSTYRVVSVLTVLHQDW | sequence) |
| | LNGKEYKCKVSNKALPAPIEKTISKAKGQPREPQVYTLPPSRDELTKNQV | |
| | SLTCLVKGFYPSDIAVEWESNGQPENNYKTTPPVLDSDGSFFLYSKLTVD | |
| | KSRWQQGNVFSCSVMHEALHNHYTQKSLSLSPGK | |
| 338 | DIQMTQSPSSLSASVGDRVTITCRASQSVSSAVAWYQQKPGKAPKLLIYS | IgG light chain |
| | ASSLYSGVSRFSGSRSGTDFTLTISSLQPEDFATYYCQQ | framework region 1 |
| 339 | TFGQGTKVEIKRTVAAPSVFIFPPSDSQLKSGTASVVCLLNNFYPREAKV | IgG light chain |
| | QWKVDNALQSGNSQESVTEQDSKDSTYSLSSTLTLSKADYEKHKVYAC | framework region 2 |
| | EVTHQGLSSPVTKSENRGEC | |
| 340 | EISEVQLVESGGGLVQPGGSLRLSCAASGF | IgG heavy chain |
| | | Fab framework |
| | | region 1 |
| 341 | WVRQAPGKGLEWV | IgG heavy chain |
| | | Fab framework |
| | | region 2 |
| 342 | YADSVKGRFTISADTSKNTAYLQMNSLRAEDTAVYYCAR | IgG heavy chain |
| | | Fab framework |
| | | region 3 |
| 343 | DYWGQGTLVTVSSASTKGPSVFPLAPSSKSTSGGTAALGCLVKDYFPEP | IgG heavy chain |
| | VTVSWNSGALTSGVHTFPAVLQSSGLYSLSSVVTVPSSSLGTQTYICNVN | Fab framework |
| | HKPSNTKVDKKVEPKSC | region 4 |
| 344 | DIQMTQSPSSLSASVGDRVTITCRASQSVGSALAWYQQKPGKAPKLLIYS | Alternative IgG |
| | ASSLYSGVPSRFSGSRSGTDFTLTISSLQPEDFATYYCQQ | light chain |
| | | framework region 1 |
| 345 | TFGQGTKVEIKRTVAAPSVFIFPPSDSQLKSGTASVVCLLNNFYPREAKV | Alternative IgG |
| | QWKVDNALQSGNSQESVTEQDSKDSTYSLSSTLTLSKADYEKHKVYAC | light chain |
| | EVTHQGLSSPVTKSFNRGEC | framework region 2 |
| 346 | EVQLVESGGGLVQPGGSLRLSCAASGFN | Alternative IgG |
| | | heavy chain Fab |
| | | framework region 1 |
| 347 | HWVRQAPGKGLEWVA | Alternative IgG |
| | | heavy chain Fab |
| | | framework region 2 |
| 348 | YADSVKGRFTISADTSKNTAYLQMNSLRAEDTAVYYCAR | Alternative IgG |
| | | heavy chain Fab |
| | | framework region 3 |
| 349 | DYWGQGTLVTVSSASTKGPSVFPLAPSSKSTSGGTAALGCLVKDYFPEP | Alternative IgG |
| | VTVSWNSGALTSGVHTFPAVLQSSGLYSLSSVVTVPSSSLGTQTYICNVN | heavy chain Fab |
| | HKPSNTKVDKKVEPKSC | framework region 4 |
| 350 | EVQLVESGGGLVQPGGSLRLSCAASGF | VH binder |
| | | framework region 1 |
| 351 | IGWVRRAPGKGEELVA | VH binder |
| | | framework region 2 |
| 352 | YADSVKGRFTISADTSKNTAYLQMNSLRAEDTAVYYCAR | VH binder |
| | | framework region 3 |
| 353 | DYWGQGTLVTVSS | VH binder |
| | | framework region 4 |

1. A bispecific binding agent comprising:
a) a first binding domain that specifically binds to a E3 ligase; and
b) a second binding domain that specifically binds to an extracellular epitope on a target protein of a target cell, wherein both the E3 ligase and the target protein are membrane associated.

2. The bispecific binding agent of claim 1, wherein binding of the bispecific binding agent to both the E3 ligase and the target protein results in ubiquitination of the target protein.

3. The bispecific binding agent of claim 1 or 2, wherein the target cell is a neoplastic cell.

4. The bispecific binding agent of any one of claims 1 to 3, wherein the cell is a cancer cell selected from the group consisting of breast cancer, B cell lymphoma, pancreatic cancer, Hodgkin's lymphoma, ovarian cancer, prostate cancer, mesothelioma, lung cancer, non-Hodgkin's B-cell (B-NHL), melanoma, chronic lymphocytic leukemia, acute lymphocytic leukemia, neuroblastoma, glioma, glioblastoma, bladder cancer, and colorectal cancer.

5. The bispecific binding agent of any one of claims 1 to 4, wherein the target protein is an immune checkpoint protein.

6. The bispecific binding agent of any one of claims 1 to 5, wherein the target protein is selected from the group consisting of PD-L1, PD-1, CTLA-4, A2AR, B7-H3, B7-H4, BTLA, KIR, LAG3, NKG2D, TIM-3, VISTA, and SIGLEC7.

7. The bispecific binding agent of any one of claims 1 to 6, wherein the first binding domain specifically binds to an extracellular protein attached to an E3 ligase or a transmembrane protein that interacts with an E3 ligase.

8. The bispecific binding agent of any one of claims 1 to 7, wherein degradation of the target protein reduces the ability of the target cell to proliferate.

9. The bispecific binding agent of any one of claims 1 to 8, wherein the target protein is selected from the group consisting of HER2, CD19, CD20, CDCP1, PD-L1, EGFR, MMP14, and CTLA-4.

10. The bispecific binding agent of any one of claims 1 to 9, wherein the E3 ligase is a transmembrane protein.

11. The bispecific binding agent of any one of claims 1 to 10, wherein the E3 ligase is selected from the group consisting of RNF43, RNF128 (GRAIL), ZNRF3, and MARCH11.

12. The bispecific binding agent of any one of claims 1 to 11, wherein the first binding domain and the second binding domain are each independently selected from the group consisting of half antibodies, single-domain antibodies, nanobodies, monospecific Fab₂, scFv, scFv-Fc, minibodies, IgNAR, V-NAR, hcIgG, VhH, camelid antibodies, and peptibodies, or the first binding domain and the second binding domain together form a bispecific antibody, a bispecific diabody, a bispecific Fab₂, a bispecific camelid antibody, or a bispecific peptibody.

13. The bispecific binding agent of any one of claims 1 to 12, wherein
i). the first binding domain comprises heavy chain framework region (FR) sequence set forth in SEQ ID NOs.: 12 or 320 and light chain FR sequence set forth in SEQ ID NOs.: 11 or 319; and
ii). the second binding domain comprises heavy chain FR sequence set forth in SEQ ID NOs.: 12 or 320 and light chain FR sequence set forth in SEQ ID NOs.: 11 or 319.

14. The bispecific binding agent of any one of claims 1 to 13, wherein
i). the first binding domain comprises light chain variable domain CDR3 (LC-CDR3) sequence and heavy chain variable domain CDR1 (HC-CDR1), HC-CDR2, and HC-CDR3 sequences comprising the sequences set forth in Table 2, respectively; and
ii). the second binding domain comprises LC-CDR3 sequence and HC-CDR1, HC-CDR2, and HC-CDR3 sequences comprising the sequences set forth in Table 3, respectively.

15. The bispecific binding agent of any one of claims 1 to 14, wherein
i). the first binding domain comprises a heavy chain variable domain (VH), and wherein the VH comprises the FR sequence set forth in SEQ ID NO.: 321; and
ii). the second binding domain comprises heavy chain FR sequence set forth in SEQ ID NOs.: 12 or 320 and light chain FR sequence set forth in SEQ ID NOs.: 11 or 319.

16. The bispecific binding agent of claim 15, wherein
i). the first binding domain comprises VH-CDR1, VH-CDR2, and VH-CDR3 sequences set forth in Table 4, respectively; and
ii). the second binding domain comprises LC-CDR3 sequence and HC-CDR1, HC-CDR2, and HC-CDR3 sequences comprising the sequences set forth in Table 3, respectively.

17. The bispecific binding agent of any one of claims 1 to 16, wherein the bispecific binding agent comprises a bispecific antibody.

18. The bispecific binding agent of any one of claims 1 to 17, wherein the bispecific binding agent comprises a bispecific IgG.

19. The bispecific binding agent of any one of claims 1 to 18, wherein the bispecific binding agent comprises a knob and hole bispecific IgG.

20. The bispecific binding agent of any one of claims 1 to 19, wherein the first binding domain comprises a bispecific antibody, and the second binding domain comprises a single chain Fab.

21. The bispecific binding agent of any one of claims 1 to 20, wherein the first binding domain comprises a Fab, and the second binding domain comprises an scFv.

22. A nucleic acid that encodes the bispecific binding agent of any one of claims 1 to 21.

23. The nucleic acid of claim 22, wherein the nucleic acid is operably connected to a promoter.

24. An engineered cell capable of protein expression comprising the nucleic acid of claim 22 or 23.

25. The cell of claim 23, wherein the cell is a B cell, a B memory cell, or a plasma cell.

26. A method for making a bispecific binding agent, the method comprising:
a) providing a cell capable of protein synthesis, comprising the nucleic acid of claim 20 or 21; and
b) inducing expression of the bispecific binding agent.

27. A vector, comprising the nucleic acid of claim 22 or 23.

28. The vector of claim 24, further comprising a promoter, wherein the promoter is operably linked to the nucleic acid.

29. An immunoconjugate comprising:
i). a bispecific binding agent of any one of claims 1 to 21,
ii). a small molecule, and
iii). a linker.

30. The immunoconjugate of claim 29, wherein the linker is selected from the group consisting of a cleavable linker, a non-cleavable linker, a hydrophilic linker, and a dicarboxylic acid based linker.

31. The immunoconjugate of claim 29 or 30, wherein the linker is a PEG4.

32. The immunoconjugate of any one of claims 29 to 31, wherein the small molecule comprises amine, CGS21680, oxaziridine-azide, ZM241385, plerixafor, maraviroc, and aplaviroc.

33. A pharmaceutical composition, comprising: (1) the bispecific binding agent of any one of claims 1 to 21, the nucleic acid of claim 22 or 23, or the immunoconjugate of any one of claims 29 to 32, and (2) a pharmaceutically acceptable carrier.

34. An engineered transmembrane protein for the treatment of neoplastic disease in which a target protein is present on the surface of a neoplastic cell, comprising:
a) a membrane-associated E3 ligase, linked to
b) a target protein binding domain specific for the target protein.

35. The engineered transmembrane protein of claim 34, wherein the E3 ligase and the target protein binding domain are covalently linked.

36. The engineered transmembrane protein of claim 34 or 35, wherein the E3 ligase and the target protein binding domain are expressed as a fusion protein.

37. The engineered transmembrane protein of any one of claim 34 or 36, wherein the E3 ligase and the target protein binding domain are covalently linked by a disulfide bond.

38. The engineered transmembrane protein of any one of claims 34 to 37, wherein the target protein binding domain is specific for a target protein is selected from the group consisting of HER2, EGFR, CDCP1, PD-L1, PD-1, CTLA-4, CD19, CD20, A2AR, B7-H3, B7-H4, BTLA, KIR, LAG3, NKG2D, TIM-3, VISTA, and SIGLEC7.

39. A nucleic acid that encodes the engineered transmembrane protein of any one of claims 34 to 38.

40. The nucleic acid of claim 39, wherein the nucleic acid further comprises a vector.

41. The nucleic acid of claim 39 or 40, wherein the nucleic acid is operably connected to a promoter.

42. An engineered cell comprising the engineered transmembrane protein of any one of claims 34 to 38, or the nucleic acid of any one of claims 39 to 41.

43. A composition for the treatment of a neoplastic disease in which a target protein is present on the surface of a neoplastic cell, comprising:
a) a therapeutic amount of the engineered transmembrane protein of any one of claims 34 to 38, and
b) a fusogenic carrier, wherein the carrier is capable of fusing with the neoplastic cell plasma membrane.

44. The composition of claim 43, wherein the carrier is a fusogenic liposome.

45. A composition for the treatment of a neoplastic disease in which a target protein is present on the surface of a neoplastic cell, comprising:
a) a therapeutic amount of the nucleic acid of any one of claims 39 to 41, and
b) a pharmaceutically acceptable carrier, wherein the carrier is capable of delivering the nucleic acid to the neoplastic cell cytosol.

46. The composition of claim 45, wherein the carrier comprises a viral particle.

47. The composition of claim 45, wherein the carrier comprises a liposome.

48. A method of treating a neoplastic disease or disorder in a subject, the method comprising administering to a subject in need thereof, a therapeutically effective amount of:
a) the bispecific binding agent of any one of claims 1 to 21;
b) the immunoconjugate of any one of claims 29 to 32;
c) the engineered transmembrane protein of any one of claims 34 to 38;
d) the nucleic acid of any one of claim 22 or 23 or 39 to 41; or
e) the cell of any one of claims 24, 25, or 42.

49. A use for the treatment of neoplastic disease of:
a) the bispecific binding agent of any one of claims 1 to 21;
b) the immunoconjugate of any one of claims 29 to 32;
c) the engineered transmembrane protein of any one of claims 34 to 38;
d) the nucleic acid of any one of claim 22 or 23 or 39 to 41; or
e) the cell of any one of claims 24, 25, or 42.

50. A use for the manufacture of a medicament for the treatment of neoplastic disease of:
a) the bispecific binding agent of any one of claims 1 to 21;
b) the immunoconjugate of any one of claims 29 to 32;
c) the engineered transmembrane protein of any one of claims 34 to 38;
d) the nucleic acid of any one of claim 22 or 23 or 39 to 41; or
e) the cell of any one of claims 24, 25, or 42.


SEQUENCE LISTING (360 sequences)

SEQ ID NO: 1 - protein, 1271 aa, Artificial sequence
  Synthetic polypeptide
  misc_feature: RNF43-antiGFP scFab construct
DIQMTQSPSSLSASVGDRVTITCRASQSVSSAVAWYQQKPGKAPKLLIYSASSLYSGVPSRFSGSRSGTDFTLTISSLQPEDFATYYCQQSWGLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGECGGSSGSGSGSTGTSSSGTGTSAGTTGTSASTSGSGSGGGGGSGGGGSAGGTATAGASSGSEVQLVESGGGLVQPGGSLRLSCAASGFNISYYSIHWVRQAPGKGLEWVASIYPYYSSTSYADSVKGRFTISADTSKNTAYLQMNSLRAEDTAVYYCARAGWVASSGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGFGRTGLVLAAAVESERSAEQKAIIRVIPLKMDPTGKLNLTLEGVFAGVAEITPAEGKLMQSHPLYLCNASDDDNLEPGFISIVKLESPRRAPRPCLSLASKARMAGERGASAVLFDITEDRAAAEQLQQPLGLTWPVVLIWGNDAEKLMEFVYKNQKAHVRIELKEPPAWPDYDVWILMTVVGTIFVIILASVLRIRCRPRHSRPDPLQQRTAWAISQLATRRYQASCRQARGEWPDSGSSCSSAPVCAICLEEFSEGQELRVISCLHEFHRNCVDPWLHQHRTCPLCMFNITEGDSFSQSLGPSRSYQEPGRRLHLIRQHPGHAHYHLPAAYLLGPSRSAVARPPRPGPFLPSQEPGMGPRHHRFPRAAHPRAPGEQQRLAGAQHPYAQGWGLSHLQSTSQHPAACPVPLRRARPPDSSGSGESYCTERSGYLADGPASDSSSGPCHGSSSDSVVNCTDISLQGVHGSSSTFCSSLSSDFDPLVYCSPKGDPQRVDMQPSVTSRPRSLDSVVPTGETQVSSHVHYHRHRHHHYKKRFQWHGRKPGPETGVPQSRPPIPRTQPQPEPPSPDQQVTRSNSAAPSGRLSNPQCPRALPEPAPGPVDASSICPSTSSLFNLQKSSLSARHPQRKRRGGPSEPTPGSRPQDATVHPACQIFPHYTPSVAYPWSPEAHPLICGPPGLDKRLLPETPGPCYSNSQPVWLCLTPRQPLEPHPPGEGPSEWSSDTAEGRPCPYPHCQVLSAQPGSEEELEELCEQAV

SEQ ID NO: 2 - protein, 213 aa, Artificial sequence
  Synthetic polypeptide
  misc_feature: anti-GFP Light chain domain of SEQ ID NO1
DIQMTQSPSSLSASVGDRVTITCRASQSVSSAVAWYQQKPGKAPKLLIYSASSLYSGVPSRFSGSRSGTDFTLTISSLQPEDFATYYCQQSWGLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 60 aa, Artificial sequence
  Synthetic polypeptide
  misc_feature: scFAB linker domain of SEQ ID NO1
GGSSGSGSGSTGTSSSGTGTSAGTTGTSASTSGSGSGGGGGSGGGGSAGGTATAGASSGS

SEQ ID NO: 4 - protein, 228 aa, Artificial sequence
  Synthetic polypeptide
  misc_feature: Anti-GFP Heavy chain domain of SEQ ID NO1
EVQLVESGGGLVQPGGSLRLSCAASGFNISYYSIHWVRQAPGKGLEWVASIYPYYSSTSYADSVKGRFTISADTSKNTAYLQMNSLRAEDTAVYYCARAGWVASSGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 5 - protein, 10 aa, Artificial sequence
  Synthetic polypeptide
  misc_feature: Linker domain of SEQ ID NO1
GGGGSGGGGS

SEQ ID NO: 6 - protein, 760 aa, Artificial sequence
  Synthetic polypeptide
  misc_feature: RNF43 domain of SEQ ID NO1
GFGRTGLVLAAAVESERSAEQKAIIRVIPLKMDPTGKLNLTLEGVFAGVAEITPAEGKLMQSHPLYLCNASDDDNLEPGFISIVKLESPRRAPRPCLSLASKARMAGERGASAVLFDITEDRAAAEQLQQPLGLTWPVVLIWGNDAEKLMEFVYKNQKAHVRIELKEPPAWPDYDVWILMTVVGTIFVIILASVLRIRCRPRHSRPDPLQQRTAWAISQLATRRYQASCRQARGEWPDSGSSCSSAPVCAICLEEFSEGQELRVISCLHEFHRNCVDPWLHQHRTCPLCMFNITEGDSFSQSLGPSRSYQEPGRRLHLIRQHPGHAHYHLPAAYLLGPSRSAVARPPRPGPFLPSQEPGMGPRHHRFPRAAHPRAPGEQQRLAGAQHPYAQGWGLSHLQSTSQHPAACPVPLRRARPPDSSGSGESYCTERSGYLADGPASDSSSGPCHGSSSDSVVNCTDISLQGVHGSSSTFCSSLSSDFDPLVYCSPKGDPQRVDMQPSVTSRPRSLDSVVPTGETQVSSHVHYHRHRHHHYKKRFQWHGRKPGPETGVPQSRPPIPRTQPQPEPPSPDQQVTRSNSAAPSGRLSNPQCPRALPEPAPGPVDASSICPSTSSLFNLQKSSLSARHPQRKRRGGPSEPTPGSRPQDATVHPACQIFPHYTPSVAYPWSPEAHPLICGPPGLDKRLLPETPGPCYSNSQPVWLCLTPRQPLEPHPPGEGPSEWSSDTAEGRPCPYPHCQVLSAQPGSEEELEELCEQAV

SEQ ID NO: 7 - protein, 472 aa, Artificial sequence
  Synthetic polypeptide
  misc_feature: GFP-NanoLuc reporter construct
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYKGSGGSGGGGSAVGQDTQEVIVVPHSLPFKVVVISAILALVVLTIISLIILIMLWQKKPRGGSGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 8 - protein, 238 aa, Artificial sequence
  Synthetic polypeptide
  misc_feature: GFP domain of SEQ ID NO7
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYK

SEQ ID NO: 9 - protein, 64 aa, Artificial sequence
  Synthetic polypeptide
  misc_feature: Transmembrane and linker domains of SEQ ID NO7
GSGGSGGGGSAVGQDTQEVIVVPHSLPFKVVVISAILALVVLTIISLIILIMLWQKKPRGGSGS

SEQ ID NO: 10 - protein, 170 aa, Artificial sequence
  Synthetic polypeptide
  misc_feature: Nanoluciferase domain of SEQ ID NO7
VFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 11 - protein, 207 aa, Artificial sequence
  Synthetic polypeptide
  misc_feature: IgG light chain framework regions
  misc_feature (89)..(90): LC-CDR3
  misc_feature (101)..(207): constant domain
DIQMTQSPSSLSASVGDRVTITCRASQSVSSAVAWYQQKPGKAPKLLIYSASSLYSGVSRFSGSRSGTDFTLTISSLQPEDFATYYCQQTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 198 aa, Artificial sequence
  Synthetic polypeptide
  misc_feature: IgG heavy chain Fab framework regions
  misc_feature (30)..(31): HC-CDR1
  misc_feature (43)..(44): HC-CDR2
  misc_feature (82)..(83): HC-CDR3
  misc_feature (96)..(198): constant domain
EISEVQLVESGGGLVQPGGSLRLSCAASGFWVRQAPGKGLEWVYADSVKGRFTISADTSKNTAYLQMNSLRAEDTAVYYCARDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 13 - protein, 465 aa, Artificial sequence
  Synthetic polypeptide
  misc_feature: Bispecific IgG having a single chain Fab as one arm
  misc_feature (89)..(90): LC-CDR3
  misc_feature (101)..(207): constant domain
  misc_feature (297)..(298): HC-CDR1
  misc_feature (310)..(311): HC-CDR2
  misc_feature (349)..(350): HC-CDR3
  misc_feature (363)..(465): constant domain
DIQMTQSPSSLSASVGDRVTITCRASQSVSSAVAWYQQKPGKAPKLLIYSASSLYSGVSRFSGSRSGTDFTLTISSLQPEDFATYYCQQTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGECGGSSGSGSGSTGTSSSGTGTSAGTTGTSASTSGSGSGGGGGSGGGGSAGGTATAGASSGSEISEVQLVESGGGLVQPGGSLRLSCAASGFWVRQAPGKGLEWVYADSVKGRFTISADTSKNTAYLQMNSLRAEDTAVYYCARDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 14 - protein, 236 aa, Artificial sequence
  Synthetic polypeptide
  misc_feature: IgG heavy chain Fc portion “knob”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 15 - protein, 227 aa, Artificial sequence
  Synthetic polypeptide
  misc_feature: IgG heavy chain Fc portion “hole”
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 16 - protein, 436 aa, Artificial sequence
  Synthetic polypeptide
  misc_feature: Fab-scFv construct - heavy chain-scFv fusion
  misc_feature (30)..(31): Antigen1-HC-CDR1
  misc_feature (43)..(44): Antigen1-HC-CDR2
  misc_feature (82)..(83): Antigen1-HC-CDR3
  misc_feature (96)..(198): constant domain
  misc_feature (315)..(316): Antigen2-LC-CDR3
  misc_feature (371)..(372): Antigen2-HC-CDR1
  misc_feature (384)..(385): Antigen2-HC-CDR2
  misc_feature (423)..(424): Antigen2-HC-CDR3
EISEVQLVESGGGLVQPGGSLRLSCAASGFWVRQAPGKGLEWVYADSVKGRFTISADTSKNTAYLQMNSLRAEDTAVYYCARDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGSGSAGGLNDIFEAQKIEWHESDIQMTQSPSSLSASVGDRVTITCRASQSVSSAVAWYQQKPGKAPKLLIYSASSLYSGVSRFSGSRSGTDFTLTISSLQPEDFATYYCQQTFGQGTKVEIKGGGGSGGGGSGGGGSEISEVQLVESGGGLVQPGGSLRLSCAASGFWVRQAPGKGLEWVYADSVKGRFTISADTSKNTAYLQMNSLRAEDTAVYYCARDYWGQGTLVTVSS

SEQ ID NO: 17 - protein, 207 aa, Artificial sequence; identical to SEQ ID NO: 11
  Synthetic polypeptide
  misc_feature: Fab-scFv construct - heavy chain-scFv fusion (light chain)
  misc_feature (89)..(90): Antigen1-LC-CDR3
  misc_feature (101)..(207): constant domain

SEQ ID NO: 18 - protein, 9 aa, Artificial sequence
  Synthetic polypeptide
  misc_feature: RNF43 LC-CDR3
YYDSSYALF

SEQ ID NO: 19 - protein, 5 aa, Artificial sequence
  Synthetic polypeptide
  misc_feature: RNF43 LC-CDR3
SGWPF

SEQ ID NO: 20 - protein, 6 aa, Artificial sequence
  Synthetic polypeptide
  misc_feature: RNF43 LC-CDR3
GYSDLI

SEQ ID NO: 21 - protein, 5 aa, Artificial sequence
  Synthetic polypeptide
  misc_feature: RNF43 LC-CDR3
VYPPI

SEQ ID NO: 22 - protein, 4 aa, Artificial sequence
  Synthetic polypeptide
  misc_feature: RNF43 LC-CDR3
AYPI

SEQ ID NO: 23 - protein, 8 aa, Artificial sequence
  Synthetic polypeptide
  misc_feature: RNF43 LC-CDR3
SKYSNQLI

SEQ ID NO: 24 - protein, 7 aa, Artificial sequence
  Synthetic polypeptide
  misc_feature: RNF43 LC-CDR3
WSWPYPL

SEQ ID NO: 25 - protein, 7 aa, Artificial sequence
  Synthetic polypeptide
  misc_feature: RNF43 LC-CDR3
SSFIWPL

SEQ ID NO: 26 - protein, 7 aa, Artificial sequence
  Synthetic polypeptide
  misc_feature: RNF43 LC-CDR3
SHWEKLI

SEQ ID NO: 27 - protein, 6 aa, Artificial sequence
  Synthetic polypeptide
  misc_feature: RNF43 LC-CDR3
GRSWPV

SEQ ID NO: 28 - protein, 7 aa, Artificial sequence
  Synthetic polypeptide
  misc_feature: RNF43 LC-CDR3
KVRWPLI

SEQ ID NO: 29 - protein, 6 aa, Artificial sequence
  Synthetic polypeptide
  misc_feature: RNF43 LC-CDR3
SSKGLI

SEQ ID NO: 30 - protein, 6 aa, Artificial sequence
  Synthetic polypeptide
  misc_feature: RNF43 LC-CDR3
ALYYPI

SEQ ID NO: 31 - protein, 6 aa, Artificial sequence
  Synthetic polypeptide
  misc_feature: RNF43 LC-CDR3
SYYWPV

SEQ ID NO: 32 - protein, 7 aa, Artificial sequence
  Synthetic polypeptide
  misc_feature: RNF43 LC-CDR3
YVYSYPF

SEQ ID NO: 33 - protein, 6 aa, Artificial sequence
  Synthetic polypeptide
  misc_feature: RNF43 LC-CDR3
WWYFPI

SEQ ID NO: 34 - protein, 5 aa, Artificial sequence
  Synthetic polypeptide
  misc_feature: ZNRF3 LC-CDR3
SSSPF

SEQ ID NO: 35 - protein, 5 aa, Artificial sequence
  Synthetic polypeptide
  misc_feature: ZNRF3 LC-CDR3
SYYPI

SEQ ID NO: 36 - protein, 9 aa, Artificial sequence
  Synthetic polypeptide
  misc_feature: ZNRF3 LC-CDR3
SSYYFWSPI

SEQ ID NO: 37 - protein, 7 aa, Artificial sequence
  Synthetic polypeptide
  misc_feature: ZNRF3 LC-CDR3
YAYYSPF

SEQ ID NO: 38 - protein, 6 aa, Artificial sequence
  Synthetic polypeptide
  misc_feature: ZNRF3 LC-CDR3
SYYSLF

SEQ ID NO: 39 - protein, 6 aa, Artificial sequence
  Synthetic polypeptide
  misc_feature: ZNRF3 LC-CDR3
GWVVPI

SEQ ID NO: 40 - protein, 6 aa, Artificial sequence
  Synthetic polypeptide
  misc_feature: ZNRF3 LC-CDR3
SWDSLI

SEQ ID NO: 41 - protein, 6 aa, Artificial sequence
  Synthetic polypeptide
  misc_feature: ZNRF3 LC-CDR3
SRRYPV

SEQ ID NO: 42 - protein, 6 aa, Artificial sequence
  Synthetic polypeptide
  misc_feature: ZNRF3 LC-CDR3
YSGSLI

SEQ ID NO: 43 - protein, 6 aa, Artificial sequence
  Synthetic polypeptide
  misc_feature: ZNRF3 LC-CDR3
SPYELI

SEQ ID NO: 44 - protein, 6 aa, Artificial sequence
  Synthetic polypeptide
  misc_feature: ZNRF3 LC-CDR3
SSSDPI

SEQ ID NO: 45 - protein, 6 aa, Artificial sequence
  Synthetic polypeptide
  misc_feature: ZNRF3 LC-CDR3
SRRYPV

SEQ ID NO: 46 - protein, 8 aa, Artificial sequence
  Synthetic polypeptide
  misc_feature: ZNRF3 LC-CDR3
GYKGSSLI

SEQ ID NO: 47 - protein, 6 aa, Artificial sequence
  Synthetic polypeptide
  misc_feature: ZNRF3 LC-CDR3
SWGWPI

SEQ ID NO: 48 - protein, 8 aa, Artificial sequence
  Synthetic polypeptide
  misc_feature: ZNRF3 LC-CDR3
PYPGMQPI

SEQ ID NO: 49 - protein, 6 aa, Artificial sequence
  Synthetic polypeptide
  misc_feature: ZNRF3 LC-CDR3
MSSSPI

SEQ ID NO: 50 - protein, 6 aa, Artificial sequence
  Synthetic polypeptide
  misc_feature: ZNRF3 LC-CDR3
SSSSLI

SEQ ID NO: 51 - protein, 6 aa, Artificial sequence
  Synthetic polypeptide
  misc_feature: ZNRF3 LC-CDR3
SSHYLI

SEQ ID NO: 52 - protein, 7 aa, Artificial sequence
  Synthetic polypeptide
  misc_feature: ZNRF3 LC-CDR3
YSFSSLI

SEQ ID NO: 53 - protein, 6 aa, Artificial sequence
  Synthetic polypeptide
  misc_feature: ZNRF3 LC-CDR3
SRRYPV

SEQ ID NO: 54 - protein, 7 aa, Artificial sequence
  Synthetic polypeptide
  misc_feature: ZNRF3 LC-CDR3
TWSVVPI

SEQ ID NO: 55 - protein, 6 aa, Artificial sequence
  Synthetic polypeptide
  misc_feature: ZNRF3 LC-CDR3
SRRYPV

SEQ ID NO: 56 - protein, 7 aa, Artificial sequence
  Synthetic polypeptide
  misc_feature: GRAIL LC-CDR3
SHPAFPF

SEQ ID NO: 57 - protein, 7 aa, Artificial sequence
  Synthetic polypeptide
  misc_feature: GRAIL LC-CDR3
GGGWYPF

SEQ ID NO: 58 - protein, 6 aa, Artificial sequence
  Synthetic polypeptide
  misc_feature: GRAIL LC-CDR3
SHAYPI

SEQ ID NO: 59 - protein, 5 aa, Artificial sequence
  Synthetic polypeptide
  misc_feature: GRAIL LC-CDR3
YYYLI

SEQ ID NO: 60 - protein, 5 aa, Artificial sequence
  Synthetic polypeptide
  misc_feature: GRAIL LC-CDR3
HHSLI

SEQ ID NO: 61 - protein, 6 aa, Artificial sequence
  Synthetic polypeptide
  misc_feature: RNF43 HC-CDR1
ISYYSI

SEQ ID NO: 62 - protein, 6 aa, Artificial sequence
  Synthetic polypeptide
  misc_feature: RNF43 HC-CDR1
IYSYYM

SEQ ID NO: 63 - protein, 6 aa, Artificial sequence
  Synthetic polypeptide
  misc_feature: RNF43 HC-CDR1
IYYYSI

SEQ ID NO: 64 - protein, 6 aa, Artificial sequence
  Synthetic polypeptide
  misc_feature: RNF43 HC-CDR1
LSYSYI

SEQ ID NO: 65 - protein, 6 aa, Artificial sequence
  Synthetic polypeptide
  misc_feature: RNF43 HC-CDR1
IYYYSM

SEQ ID NO: 66 - protein, 6 aa, Artificial sequence
  Synthetic polypeptide
  misc_feature: RNF43 HC-CDR1
VSYYYI

SEQ ID NO: 67 - protein, 6 aa, Artificial sequence
  Synthetic polypeptide
  misc_feature: RNF43 HC-CDR1
FYSYSI

SEQ ID NO: 68 - protein, 6 aa, Artificial sequence
  Synthetic polypeptide
  misc_feature: RNF43 HC-CDR1
FYSYSI

SEQ ID NO: 69 - protein, 6 aa, Artificial sequence
  Synthetic polypeptide
  misc_feature: RNF43 HC-CDR1
IYSYYI

SEQ ID NO: 70 - protein, 6 aa, Artificial sequence
  Synthetic polypeptide
  misc_feature: RNF43 HC-CDR1
FYSYSI

SEQ ID NO: 71 - protein, 6 aa, Artificial sequence
  Synthetic polypeptide
  misc_feature: RNF43 HC-CDR1
IYSSSI

SEQ ID NO: 72 - protein, 6 aa, Artificial sequence
  Synthetic polypeptide
  misc_feature: RNF43 HC-CDR1
VSSSSI

SEQ ID NO: 73 - protein, 6 aa, Artificial sequence
  Synthetic polypeptide
  misc_feature: RNF43 HC-CDR1
VYSSSI

SEQ ID NO: 74 - protein, 6 aa, Artificial sequence
  Synthetic polypeptide
  misc_feature: RNF43 HC-CDR1
VYYSSI

SEQ ID NO: 75 - protein, 6 aa, Artificial sequence
  Synthetic polypeptide
  misc_feature: RNF43 HC-CDR1
IYYSYI

SEQ ID NO: 76 - protein, 6 aa, Artificial sequence
  Synthetic polypeptide
  misc_feature: RNF43 HC-CDR1
VSYSSI

SEQ ID NO: 77 - protein, 6 aa, Artificial sequence
  Synthetic polypeptide
  misc_feature: ZRNF3 HC-CDR1
ISYSSI

SEQ ID NO: 78 - protein, 6 aa, Artificial sequence
  Synthetic polypeptide
  misc_feature: ZRNF3 HC-CDR1
LYYSYI

SEQ ID NO: 79 - protein, 6 aa, Artificial sequence
  Synthetic polypeptide
  misc_feature: ZRNF3 HC-CDR1
LYYSSI

SEQ ID NO: 80 - protein, 6 aa, Artificial sequence
  Synthetic polypeptide
  misc_feature: ZRNF3 HC-CDR1
ISYYSM

SEQ ID NO: 81 - protein, 6 aa, Artificial sequence
  Synthetic polypeptide
  misc_feature: ZRNF3 HC-CDR1
LSSYSI

SEQ ID NO: 82 - protein, 6 aa, Artificial sequence
  Synthetic polypeptide
  misc_feature: ZRNF3 HC-CDR1
ISYYYM

SEQ ID NO: 83 - protein, 6 aa, Artificial sequence
  Synthetic polypeptide
  misc_feature: ZRNF3 HC-CDR1
ISSSSM

SEQ ID NO: 84 - protein, 6 aa, Artificial sequence
  Synthetic polypeptide
  misc_feature: ZRNF3 HC-CDR1
ISYSSI

SEQ ID NO: 85 - protein, 6 aa, Artificial sequence
  Synthetic polypeptide
  misc_feature: ZRNF3 HC-CDR1
VYSYYI

SEQ ID NO: 86 - protein, 6 aa, Artificial sequence
  Synthetic polypeptide
  misc_feature: ZRNF3 HC-CDR1
VYSSYI

SEQ ID NO: 87 - protein, 6 aa, Artificial sequence
  Synthetic polypeptide
  misc_feature: ZRNF3 HC-CDR1
VYSSSI

SEQ ID NO: 88 - protein, 6 aa, Artificial sequence
  Synthetic polypeptide
  misc_feature: ZRNF3 HC-CDR1
VSYSSI

SEQ ID NO: 89 - protein, 6 aa, Artificial sequence
  Synthetic polypeptide
  misc_feature: ZRNF3 HC-CDR1
VYYSSI

SEQ ID NO: 90 - protein, 6 aa, Artificial sequence
  Synthetic polypeptide
  misc_feature: ZRNF3 HC-CDR1
FSSYSI

SEQ ID NO: 91 - protein, 6 aa, Artificial sequence
  Synthetic polypeptide
  misc_feature: ZRNF3 HC-CDR1
FYYYSI

SEQ ID NO: 92 - protein, 6 aa, Artificial sequence
  Synthetic polypeptide
  misc_feature: ZRNF3 HC-CDR1
ISSSYI

SEQ ID NO: 93 - protein, 6 aa, Artificial sequence
  Synthetic polypeptide
  misc_feature: ZRNF3 HC-CDR1
FYSSYI

SEQ ID NO: 94 - protein, 6 aa, Artificial sequence
  Synthetic polypeptide
  misc_feature: ZRNF3 HC-CDR1
FYSYSI

SEQ ID NO: 95 - protein, 6 aa, Artificial sequence
  Synthetic polypeptide
  misc_feature: ZRNF3 HC-CDR1
VSSSSI

SEQ ID NO: 96 - protein, 6 aa, Artificial sequence
  Synthetic polypeptide
  misc_feature: ZRNF3 HC-CDR1
ISYSSI

SEQ ID NO: 97 - protein, 6 aa, Artificial sequence
  Synthetic polypeptide
  misc_feature: ZRNF3 HC-CDR1
VYSYYI

SEQ ID NO: 98 - protein, 6 aa, Artificial sequence
  Synthetic polypeptide
  misc_feature: ZRNF3 HC-CDR1
ISYSSI

SEQ ID NO: 99 - protein, 8 aa, Artificial sequence
  Synthetic polypeptide
  misc_feature: GRAIL HC-CDR1
NLYSYSIH

SEQ ID NO: 100 - protein, 8 aa, Artificial sequence
  Synthetic polypeptide
  misc_feature: GRAIL HC-CDR1
NIYYSSMH

SEQ ID NO: 101 - protein, 8 aa, Artificial sequence
  Synthetic polypeptide
  misc_feature: GRAIL HC-CDR1
NLYSYYMH

SEQ ID NO: 102 - protein, 8 aa, Artificial sequence
  Synthetic polypeptide
  misc_feature: GRAIL HC-CDR1
NIYYYSMH

SEQ ID NO: 103 - protein, 8 aa, Artificial sequence
  Synthetic polypeptide
  misc_feature: GRAIL HC-CDR1
NISYSSMH

SEQ ID NO: 104 - protein, 107 aa, Artificial sequence [sequence omitted]

SEQ ID NO: 105 - protein, 98 aa, Artificial sequence
  Synthetic polypeptide
  misc_feature: Constant domain CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 106 - protein, 107 aa, Artificial sequence [sequence omitted]

SEQ ID NO: 107 - protein, 118 aa, Artificial sequence [sequence omitted]

SEQ ID NO: 108 - protein, 107 aa, Artificial sequence
  Synthetic polypeptide
  misc_feature: LC variable domain, anti-HER2
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIK

SEQ ID NO: 109 - protein, 120 aa, Artificial sequence [sequence omitted]

SEQ ID NO: 110 - protein, 107 aa, Artificial sequence
  Synthetic polypeptide
  misc_feature: LC variable domain, anti-EGFR
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 111 - protein, 119 aa, Artificial sequence
  Synthetic polypeptide
  misc_feature: HC variable domain, anti-EGFR
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 112 - protein, 108 aa, Artificial sequence
  Synthetic polypeptide
  misc_feature: LC variable domain, anti-CTLA4
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 113 - protein, 118 aa, Artificial sequence
  Synthetic polypeptide
  misc_feature: HC variable domain, anti-CTLA4
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 114 - protein, 108 aa, Artificial sequence
  Synthetic polypeptide
  misc_feature: LC variable domain, anti-CDCP1
DIQMTQSPSSLSASVGDRVTITCRASQSVSSAVAWYQQKPGKAPKLLIYSASSLYSGVPSRFSGSRSGTDFTLTISSLQPEDFATYYCQQSYYYYPITFGQGTKVEIK

SEQ ID NO: 115 - protein, 119 aa, Artificial sequence
  Synthetic polypeptide
  misc_feature: HC variable domain, anti-CDCP1
EISEVQLVESGGGLVQPGGSLRLSCAASGFNLSYYYIHWVRQAPGKGLEWVASIYSSSSYTSYADSVKGRFTISADTSKNTAYLQMNSLRAEDTAVYYCARAYYGFDYWGQGTLVTVSS

SEQ ID NO: 116 - protein, 7 aa, Artificial sequence
  Synthetic polypeptide
  misc_feature: CDR, 1prime Ab LC3
WSYYGLI

SEQ ID NO: 117 - protein, 7 aa, Artificial sequence
  Synthetic polypeptide
  misc_feature: CDR, 1prime Ab HC1
LSSYYIH

SEQ ID NO: 118 - protein, 10 aa, Artificial sequence
  Synthetic polypeptide
  misc_feature: CDR, 1prime Ab HC2
SIYSSSSYTS

SEQ ID NO: 119 - protein, 19 aa, Artificial sequence
  Synthetic polypeptide
  misc_feature: CDR, 1prime Ab HC3
SHGPVYSGYWYYYWYWGFD

SEQ ID NO: 120 - protein, 10 aa, Artificial sequence
  Synthetic polypeptide
  misc_feature: RNF43 HC-CDR2
SIYPYYGYTY

SEQ ID NO: 121 - protein, 10 aa, Artificial sequence
  Synthetic polypeptide
  misc_feature: RNF43 HC-CDR2
YISPYYSYTY

SEQ ID NO: 122 - protein, 10 aa, Artificial sequence
  Synthetic polypeptide
  misc_feature: RNF43 HC-CDR2
SIYSSSGYTS

SEQ ID NO: 123 - protein, 10 aa, Artificial sequence
  Synthetic polypeptide
  misc_feature: RNF43 HC-CDR2
SISPSYGYTY

SEQ ID NO: 124 - protein, 10 aa, Artificial sequence
  Synthetic polypeptide
  misc_feature: RNF43 HC-CDR2
SISPYYSYTS

SEQ ID NO: 125 - protein, 10 aa, Artificial sequence
  Synthetic polypeptide
  misc_feature: RNF43 HC-CDR2
SISSYYGSTS

SEQ ID NO: 126 - protein, 10 aa, Artificial sequence
  Synthetic polypeptide
  misc_feature: RNF43 HC-CDR2
SISSSSGSTS

SEQ ID NO: 127 - protein, 10 aa, Artificial sequence
  Synthetic polypeptide
  misc_feature: RNF43 and ZNRF3 HC-CDR2
SISPYYGSTS

SEQ ID NO: 128 - protein, 10 aa, Artificial sequence
  Synthetic polypeptide
  misc_feature: RNF43 HC-CDR2
SIYSYYGSTY

SEQ ID NO: 129 - protein, 10 aa, Artificial sequence
  Synthetic polypeptide
  misc_feature: RNF43 and ZNRF3 HC-CDR2
SISSYYGSTS

SEQ ID NO: 130 - protein, 10 aa, Artificial sequence
  Synthetic polypeptide
  misc_feature: RNF43 and ZNRF3 HC-CDR2
SISSYYGYTS

SEQ ID NO: 131 - protein, 10 aa, Artificial sequence
  Synthetic polypeptide
  misc_feature: RNF43 HC-CDR2
SISSYSGYTY

SEQ ID NO: 132 - protein, 10 aa, Artificial sequence
  Synthetic polypeptide
  misc_feature: RNF43 HC-CDR2
YISSYSGSTY

SEQ ID NO: 133 - protein, 10 aa, Artificial sequence
  Synthetic polypeptide
  misc_feature: RNF43 and ZNRF3 HC-CDR2
SISSYYSYTS

SEQ ID NO: 134 - protein, 10 aa, Artificial sequence
  Synthetic polypeptide
  misc_feature: RNF43 HC-CDR2
SISSYSGYTS

SEQ ID NO: 135 - protein, 10 aa, Artificial sequence
  Synthetic polypeptide
  misc_feature: ZNRF3 HC-CDR2
YISSYYGSTS

SEQ ID NO: 136 - protein, 10 aa, Artificial sequence
  Synthetic polypeptide
  misc_feature: ZNRF3 and PD-L1 HC-CDR2
SIYPSYGSTY

SEQ ID NO: 137 - protein, 10 aa, Artificial sequence
  Synthetic polypeptide
  misc_feature: ZNRF3 HC-CDR2
SISPSYSYTS

SEQ ID NO: 138 - protein, 10 aa, Artificial sequence
  Synthetic polypeptide
  misc_feature: ZNRF3 and PD-L1 HC-CDR2
SISPYYGYTS

SEQ ID NO: 139 - protein, 10 aa, Artificial sequence
  Synthetic polypeptide
  misc_feature: ZNRF3 and GRAIL HC-CDR2
SIYPYYSSTY

SEQ ID NO: 140 - protein, 10 aa, Artificial sequence
  Synthetic polypeptide
  misc_feature: ZNRF3 HC-CDR2
YIYPYYGSTS

SEQ ID NO: 141 - protein, 10 aa, Artificial sequence
  Synthetic polypeptide
  misc_feature: ZNRF3 HC-CDR2
SIYSYYSSTS

SEQ ID NO: 142 - protein, 10 aa, Artificial sequence
  Synthetic polypeptide
  misc_feature: ZNRF3 HC-CDR2
SISPSYGSTY

SEQ ID NO: 143 - protein, 10 aa, Artificial sequence
  Synthetic polypeptide
  misc_feature: ZNRF3 HC-CDR2
SISSYYSSTS

SEQ ID NO: 144 - protein, 10 aa, Artificial sequence
  Synthetic polypeptide
  misc_feature: ZNRF3 HC-CDR2
SISPYYGSTY

SEQ ID NO: 145 - protein, 10 aa, Artificial sequence
  Synthetic polypeptide
  misc_feature: ZNRF3 HC-CDR2
SIYSYYGSTS

SEQ ID NO: 146 - protein, 10 aa, Artificial sequence
  Synthetic polypeptide
  misc_feature: ZNRF3 HC-CDR2
SIYSYYGYTS

SEQ ID NO: 147 - protein, 10 aa, Artificial sequence
  Synthetic polypeptide
  misc_feature: ZNRF3 HC-CDR2
SISPYSGYTS

SEQ ID NO: 148 - protein, 10 aa, Artificial sequence
  Synthetic polypeptide
  misc_feature: GRAIL HC-CDR2
SISSSYGYTY

SEQ ID NO: 149 - protein, 10 aa, Artificial sequence
  Synthetic polypeptide
  misc_feature: GRAIL and PD-L1 HC-CDR2
SIYPYYGSTY

SEQ ID NO: 150 - protein, 10 aa, Artificial sequence
  Synthetic polypeptide
  misc_feature: GRAIL HC-CDR2
SISPYSRYTS

SEQ ID NO: 151 - protein, 10 aa, Artificial sequence
  Synthetic polypeptide
  misc_feature: GRAIL HC-CDR2
SIYPYYSYTY

SEQ ID NO: 152 - protein, 7 aa, Artificial sequence
  Synthetic polypeptide
  misc_feature: RNF43 HC-CDR3
GSYFYGM

SEQ ID NO: 153 - protein, 18 aa, Artificial sequence
  Synthetic polypeptide
  misc_feature: RNF43 HC-CDR3
AYADSWPGYSWGSSDFAL

SEQ ID NO: 154 - protein, 9 aa, Artificial sequence
  Synthetic polypeptide
  misc_feature: RNF43 HC-CDR3
YPYWYFDGF

SEQ ID NO: 155 - protein, 18 aa, Artificial sequence
  Synthetic polypeptide
  misc_feature: RNF43 HC-CDR3
PYHPFGGHYWWPYYYHGL

SEQ ID NO: 156 - protein, 14 aa, Artificial sequence
  Synthetic polypeptide
  misc_feature: RNF43 HC-CDR3
YGYYGWDYHRYSAF

SEQ ID NO: 157 - protein, 6 aa, Artificial sequence
  Synthetic polypeptide
  misc_feature: RNF43 HC-CDR3
EYYFGL

SEQ ID NO: 158 - protein, 13 aa, Artificial sequence
  Synthetic polypeptide
  misc_feature: RNF43 HC-CDR3
WSWYNHGSSSWAM

SEQ ID NO: 159 - protein, 12 aa, Artificial sequence
  Synthetic polypeptide
  misc_feature: RNF43 HC-CDR3
WSYWYSSYYGAM

SEQ ID NO: 160 - protein, 6 aa, Artificial sequence
  Synthetic polypeptide
  misc_feature: RNF43 HC-CDR3
IFAMGL

SEQ ID NO: 161 - protein, 7 aa, Artificial sequence
  Synthetic polypeptide
  misc_feature: RNF43 HC-CDR3
NGYNWGM

SEQ ID NO: 162 - protein, 9 aa, Artificial sequence
  Synthetic polypeptide
  misc_feature: RNF43 HC-CDR3
SYWQSYMAM

SEQ ID NO: 163 - protein, 16 aa, Artificial sequence
  Synthetic polypeptide
  misc_feature: RNF43 HC-CDR3
DIQMDSGYKWHPWLGM

SEQ ID NO: 164 - protein, 16 aa, Artificial sequence
  Synthetic polypeptide
  misc_feature: RNF43 HC-CDR3
SPYGHWYGYYGRQGGL

SEQ ID NO: 165 - protein, 12 aa, Artificial sequence
  Synthetic polypeptide
  misc_feature: RNF43 HC-CDR3
YYFYHSYGSYAL

SEQ ID NO: 166 - protein, 6 aa, Artificial sequence
  Synthetic polypeptide
  misc_feature: RNF43 HC-CDR3
EWYVGM

SEQ ID NO: 167 - protein, 7 aa, Artificial sequence
  Synthetic polypeptide
  misc_feature: RNF43 HC-CDR3
SYSYTGM

SEQ ID NO: 168 - protein, 12 aa, Artificial sequence
  Synthetic polypeptide
  misc_feature: ZNRF3 HC-CDR3
GWYPYSYSRDAM

SEQ ID NO: 169 - protein, 4 aa, Artificial sequence
  Synthetic polypeptide
  misc_feature: A18 HC-CDR3
GYAI

SEQ ID NO: 170 - protein, 8 aa, Artificial sequence
  Synthetic polypeptide
  misc_feature: A19 HC-CDR3
SWVYSWGM

SEQ ID NO: 171 - protein, 16 aa, Artificial sequence
  Synthetic polypeptide
  misc_feature: A20 HC-CDR3
WVGYYPPYYFSGSYGM

SEQ ID NO: 172 - protein, 13 aa, Artificial sequence
  Synthetic polypeptide
  misc_feature: A21 HC-CDR3
RYSYSYWGFHPAF

SEQ ID NO: 173 - protein, 11 aa, Artificial sequence
  Synthetic polypeptide
  misc_feature: A22 HC-CDR3
DVDWPYYFYAI

SEQ ID NO: 174 - protein, 18 aa, Artificial sequence
  Synthetic polypeptide
  misc_feature: A23 HC-CDR3
GAYGAPFYYYYFWWDRGM

SEQ ID NO: 175 - protein, 17 aa, Artificial sequence
  Synthetic polypeptide
  misc_feature: ZNRF3 HC-CDR3
NSSYPYSWGSKYSWLAL

SEQ ID NO: 176 - protein, 14 aa, Artificial sequence
  Synthetic polypeptide
  misc_feature: ZNRF3 HC-CDR3
SGWGWLYYWYPHGI

SEQ ID NO: 177 - protein, 7 aa, Artificial sequence
  Synthetic polypeptide
  misc_feature: ZNRF3 HC-CDR3
SSIYYAM

SEQ ID NO: 178 - protein, 17 aa, Artificial sequence
  Synthetic polypeptide
  misc_feature: ZNRF3 HC-CDR3
SIQLAKWGYYWIGSSGM

SEQ ID NO: 179 - protein, 17 aa, Artificial sequence
  Synthetic polypeptide
  misc_feature: ZNRF3 HC-CDR3
YKVYHWPVQWQRYWPAM

SEQ ID NO: 180 - protein, 12 aa, Artificial sequence
  Synthetic polypeptide
  misc_feature: ZNRF3 HC-CDR3
QSMSYWSRQYGF

SEQ ID NO: 181 - protein, 13 aa, Artificial sequence
  Synthetic polypeptide
  misc_feature: ZNRF3 HC-CDR3
DWYYVSGYYFSAF

SEQ ID NO: 182 - protein, 12 aa, Artificial sequence
  Synthetic polypeptide
  misc_feature: ZNRF3 HC-CDR3
QPWMYWWLKYAI

SEQ ID NO: 183 - protein, 16 aa, Artificial sequence
  Synthetic polypeptide
  misc_feature: ZNRF3 HC-CDR3
SWWEYFYPYGWYQYAI

SEQ ID NO: 184 - protein, 17 aa, Artificial sequence
  Synthetic polypeptide
  misc_feature: ZNRF3 HC-CDR3
KPWYSERFYQGIHYTAM

SEQ ID NO: 185 - protein, 13 aa, Artificial sequence
  Synthetic polypeptide
  misc_feature: ZNRF3 HC-CDR3
SWYPQYDWRYYAL

SEQ ID NO: 186 - protein, 16 aa, Artificial sequence
  Synthetic polypeptide
  misc_feature: ZNRF3 HC-CDR3
EEWYSSGMWWYSYGGI

SEQ ID NO: 187 - protein, 12 aa, Artificial sequence
  Synthetic polypeptide
  misc_feature: ZNRF3 HC-CDR3
YYWGYKGHYPAI

SEQ ID NO: 188 - protein, 15 aa, Artificial sequence
  Synthetic polypeptide
  misc_feature: GRAIL HC-CDR3
TVRGSKKPYFSGWAM

SEQ ID NO: 189 - protein, 9 aa, Artificial sequence
  Synthetic polypeptide
  misc_feature: GRAIL HC-CDR3
HHSYFFGGL

SEQ ID NO: 190 - protein, 13 aa, Artificial sequence
  Synthetic polypeptide
  misc_feature: GRAIL HC-CDR3
DPYFSHVFSYWGF

SEQ ID NO: 191 - protein, 16 aa, Artificial sequence
  Synthetic polypeptide
  misc_feature: GRAIL HC-CDR3
DFFSSYYPVVAASAGI

SEQ ID NO: 192 - protein, 13 aa, Artificial sequence
  Synthetic polypeptide
  misc_feature: GRAIL HC-CDR3
DSPYSYYSPWGGM

SEQ ID NO: 193 - protein, 5 aa, Artificial sequence
  Synthetic polypeptide
  misc_feature: PD-L1 LC-CDR3
YYHPI

SEQ ID NO: 194 - protein, 5 aa, Artificial sequence
  Synthetic polypeptide
  misc_feature: PD-L1 LC-CDR3
SYYPF

SEQ ID NO: 195 - protein, 6 aa, Artificial sequence
  Synthetic polypeptide
  misc_feature: PD-L1 LC-CDR3
AYYSPI

SEQ ID NO: 196 - protein, 6 aa, Artificial sequence
  Synthetic polypeptide
  misc_feature: PD-L1 LC-CDR3
HYTTPP

SEQ ID NO: 197 - protein, 5 aa, Artificial sequence
  Synthetic polypeptide
  misc_feature: PD-L1 LC-CDR3
AWGPF

SEQ ID NO: 198 - protein, 6 aa, Artificial sequence
  Synthetic polypeptide
  misc_feature: PD-L1 LC-CDR3
SSPYLF

SEQ ID NO: 199 - protein, 8 aa, Artificial sequence
  Synthetic polypeptide
  misc_feature: PD-L1 LC-CDR3
YRYADALI

SEQ ID NO: 200 - protein, 7 aa, Artificial sequence
  Synthetic polypeptide
  misc_feature: PD-L1 LC-CDR3
SRYVSPI

SEQ ID NO: 201 - protein, 6 aa, Artificial sequence
  Synthetic polypeptide
  misc_feature: PD-L1 LC-CDR3
YGSYPI

SEQ ID NO: 202 - protein, 10 aa, Artificial sequence
  Synthetic polypeptide
  misc_feature: PD-L1 LC-CDR3
DYYPYWHAPF

SEQ ID NO: 203 - protein, 5 aa, Artificial sequence
  Synthetic polypeptide
  misc_feature: PD-L1 LC-CDR3
PYSLI

SEQ ID NO: 204 - protein, 8 aa, Artificial sequence
  Synthetic polypeptide
  misc_feature: PD-L1 LC-CDR3
YWWPGSLI

SEQ ID NO: 205 - protein, 6 aa, Artificial sequence
  Synthetic polypeptide
  misc_feature: PD-L1 LC-CDR3
SSWEPV

SEQ ID NO: 206 - protein, 6 aa, Artificial sequence
  Synthetic polypeptide
  misc_feature: PD-L1 LC-CDR3
SYSYLV

SEQ ID NO: 207 - protein, 6 aa, Artificial sequence
  Synthetic polypeptide
  misc_feature: PD-L1 LC-CDR3
SMYYLI

SEQ ID NO: 208 - protein, 7 aa, Artificial sequence
  Synthetic polypeptide
  misc_feature: PD-L1 LC-CDR3
SWPGYPI

SEQ ID NO: 209 - protein, 6 aa, Artificial sequence
  Synthetic polypeptide
  misc_feature: PD-L1 LC-CDR3
DYFGLI

SEQ ID NO: 210 - protein, 7 aa, Artificial sequence
  Synthetic polypeptide
  misc_feature: PD-L1 LC-CDR3
SSWWSPI

SEQ ID NO: 211 - protein, 7 aa, Artificial sequence
  Synthetic polypeptide
  misc_feature: PD-L1 LC-CDR3
SWPGSPV

SEQ ID NO: 212 - protein, 6 aa, Artificial sequence
  Synthetic polypeptide
  misc_feature: PD-L1 LC-CDR3
GGYWLV

SEQ ID NO: 213 - protein, 6 aa, Artificial sequence
  Synthetic polypeptide
  misc_feature: PD-L1 LC-CDR3
HYQPLI

SEQ ID NO: 214 - protein, 7 aa, Artificial sequence
  Synthetic polypeptide
  misc_feature: PD-L1 LC-CDR3
PIWYEPI

SEQ ID NO: 215 - protein, 7 aa, Artificial sequence
  Synthetic polypeptide
  misc_feature: PD-L1 LC-CDR3
SWPQYPV

SEQ ID NO: 216 - protein, 6 aa, Artificial sequence
  Synthetic polypeptide
  misc_feature: PD-L1 LC-CDR3
WNYALI

SEQ ID NO: 217 - protein, 7 aa, Artificial sequence
  Synthetic polypeptide
  misc_feature: PD-L1 LC-CDR3
YNWSQLI

SEQ ID NO: 218 - protein, 8 aa, Artificial sequence
  Synthetic polypeptide
  misc_feature: PD-L1 LC-CDR3
YHWPSELF

SEQ ID NO: 219 - protein, 7 aa, Artificial sequence
  Synthetic polypeptide
  misc_feature: PD-L1 LC-CDR3
SGQYWPF

SEQ ID NO: 220 - protein, 7 aa, Artificial sequence
  Synthetic polypeptide
  misc_feature: PD-L1 LC-CDR3
SASWEPV

SEQ ID NO: 221 - protein, 6 aa, Artificial sequence
  Synthetic polypeptide
  misc_feature: PD-L1 LC-CDR3
SYYYLV

SEQ ID NO: 222 - protein, 7 aa, Artificial sequence
  Synthetic polypeptide
  misc_feature: PD-L1 LC-CDR3
SYPHSLI

SEQ ID NO: 223 - protein, 6 aa, Artificial sequence
  Synthetic polypeptide
  misc_feature: PD-L1 LC-CDR3
SSYSLF

SEQ ID NO: 224 - protein, 6 aa, Artificial sequence
  Synthetic polypeptide
  misc_feature: PD-L1 LC-CDR3
YNLSLV

SEQ ID NO: 225 - protein, 6 aa, Artificial sequence
  Synthetic polypeptide
  misc_feature: PD-L1 HC-CDR1
LSYYSI

SEQ ID NO: 226 - protein, 6 aa, Artificial sequence
  Synthetic polypeptide
  misc_feature: PD-L1 HC-CDR1
LYSYYI

SEQ ID NO: 227 - protein, 6 aa, Artificial sequence
  Synthetic polypeptide
  misc_feature: PD-L1 HC-CDR1
LSYSYM

SEQ ID NO: 228 - protein, 6 aa, Artificial sequence
  Synthetic polypeptide
  misc_feature: PD-L1 HC-CDR1
IKDTYI

SEQ ID NO: 229 - protein, 6 aa, Artificial sequence
  Synthetic polypeptide
  misc_feature: PD-L1 HC-CDR1
LSSSYM

SEQ ID NO: 230 - protein, 6 aa, Artificial sequence
  Synthetic polypeptide
  misc_feature: PD-L1 HC-CDR1
ISSYYI

SEQ ID NO: 231 - protein, 6 aa, Artificial sequence
  Synthetic polypeptide
  misc_feature: PD-L1 HC-CDR1
ISSYSI

SEQ ID NO: 232 - protein, 6 aa, Artificial sequence
  Synthetic polypeptide
  misc_feature: PD-L1 HC-CDR1
LSSSYI

SEQ ID NO: 233 - protein, 6 aa, Artificial sequence
  Synthetic polypeptide
  misc_feature: PD-L1 HC-CDR1
ISSYSM

SEQ ID NO: 234 - protein, 6 aa, Artificial sequence
  Synthetic polypeptide
  misc_feature: PD-L1 HC-CDR1
VSSYSI

SEQ ID NO: 235 - protein, 6 aa, Artificial sequence
  Synthetic polypeptide
  misc_feature: PD-L1 HC-CDR1
IYYYYI

SEQ ID NO: 236 - protein, 6 aa, Artificial sequence
  Synthetic polypeptide
  misc_feature: PD-L1 HC-CDR1
FSSSSI

SEQ ID NO: 237 - protein, 6 aa, Artificial sequence
  Synthetic polypeptide
  misc_feature: PD-L1 HC-CDR1
FSYSSI

SEQ ID NO: 238 - protein, 6 aa, Artificial sequence
  Synthetic polypeptide
  misc_feature: PD-L1 HC-CDR1
LSYSSI

SEQ ID NO: 239 - protein, 6 aa, Artificial sequence
  Synthetic polypeptide
  misc_feature: PD-L1 HC-CDR1
ISSSSI

SEQ ID NO: 240 - protein, 6 aa, Artificial sequence
  Synthetic polypeptide
  misc_feature: PD-L1 HC-CDR1
LSSSSI

SEQ ID NO: 241 - protein, 6 aa, Artificial sequence
  Synthetic polypeptide
  misc_feature: PD-L1 HC-CDR1
FSYSYI

SEQ ID NO: 242 - protein, 6 aa, Artificial sequence
  Synthetic polypeptide
  misc_feature: PD-L1 HC-CDR1
IYYSSI

SEQ ID NO: 243 - protein, 10 aa, Artificial sequence
  Synthetic polypeptide
  misc_feature: PD-L1 HC-CDR2
SISPSYGYTS

SEQ ID NO: 244 - protein, 10 aa, Artificial sequence
  Synthetic polypeptide
  misc_feature: PD-L1 HC-CDR2
SISPSSSYTY

SEQ ID NO: 245 - protein, 10 aa, Artificial sequence
  Synthetic polypeptide
  misc_feature: PD-L1 HC-CDR2
RIYPTNGYTR

SEQ ID NO: 246 - protein, 10 aa, Artificial sequence
  Synthetic polypeptide
  misc_feature: PD-L1 HC-CDR2
SIYSYSGYTS

SEQ ID NO: 247 - protein, 10 aa, Artificial sequence
  Synthetic polypeptide
  misc_feature: PD-L1 HC-CDR2
SIYPYSSYTS

SEQ ID NO: 248 - protein, 10 aa, Artificial sequence
  Synthetic polypeptide
  misc_feature: PD-L1 HC-CDR2
SISSSSSYTS

SEQ ID NO: 249 - protein, 10 aa, Artificial sequence
  Synthetic polypeptide
  misc_feature: PD-L1 HC-CDR2
SIYPSSSYTS

SEQ ID NO: 250 - protein, 10 aa, Artificial sequence
  Synthetic polypeptide
  misc_feature: PD-L1 HC-CDR2
SISSSYGYTS

SEQ ID NO: 251 - protein, 10 aa, Artificial sequence
  Synthetic polypeptide
  misc_feature: PD-L1 HC-CDR2
YISPYSSYTS

SEQ ID NO: 252 - protein, 10 aa, Artificial sequence
  Synthetic polypeptide
  misc_feature: PD-L1 HC-CDR2
SIYSYSGYTY

SEQ ID NO: 253 - protein, 10 aa, Artificial sequence
  Synthetic polypeptide
  misc_feature: PD-L1 HC-CDR2
SIYSSSGSTS

SEQ ID NO: 254 - protein, 10 aa, Artificial sequence
  Synthetic polypeptide
  misc_feature: PD-L1 HC-CDR2
YISSSSGSTS

SEQ ID NO: 255 - protein, 10 aa, Artificial sequence
  Synthetic polypeptide
  misc_feature: PD-L1 HC-CDR2
SIYPYSGYTS

SEQ ID NO: 256 - protein, 10 aa, Artificial sequence
  Synthetic polypeptide
  misc_feature: PD-L1 HC-CDR2
YIYSYSGSTY

SEQ ID NO: 257 - protein, 10 aa, Artificial sequence
  Synthetic polypeptide
  misc_feature: PD-L1 HC-CDR2
SISSYYGSTY

SEQ ID NO: 258 - protein, 10 aa, Artificial sequence
  Synthetic polypeptide
  misc_feature: PD-L1 HC-CDR2
YISSYSGSTS

SEQ ID NO: 259 - protein, 10 aa, Artificial sequence
  Synthetic polypeptide
  misc_feature: PD-L1 HC-CDR2
SIYSSYGSTS

SEQ ID NO: 260 - protein, 10 aa, Artificial sequence
  Synthetic polypeptide
  misc_feature: PD-L1 HC-CDR2
SIYSSYGYTS

SEQ ID NO: 261 - protein, 10 aa, Artificial sequence
  Synthetic polypeptide
  misc_feature: PD-L1 HC-CDR2
SISPSSSYTS

SEQ ID NO: 262 - protein, 10 aa, Artificial sequence
  Synthetic polypeptide
  misc_feature: PD-L1 HC-CDR2
SISSYYGYTY

SEQ ID NO: 263 - protein, 10 aa, Artificial sequence
  Synthetic polypeptide
  misc_feature: PD-L1 HC-CDR2
SISSYSGSTS

SEQ ID NO: 264 - protein, 13 aa, Artificial sequence
  Synthetic polypeptide
  misc_feature: PD-L1 HC-CDR3
SPWDPWAHHGHGI

SEQ ID NO: 265 - protein, 17 aa, Artificial sequence
  Synthetic polypeptide
  misc_feature: PD-L1 HC-CDR3
GVASYYYSASYSWYGGM

SEQ ID NO: 266 - protein, 11 aa, Artificial sequence
  Synthetic polypeptide
  misc_feature: PD-L1 HC-CDR3
YYYWHYFWDAF

SEQ ID NO: 267 - protein, 18 aa, Artificial sequence
  Synthetic polypeptide
  misc_feature: PD-L1 HC-CDR3
SPYAPGYFAVHGAPVGGM

SEQ ID NO: 268 - protein, 9 aa, Artificial sequence
  Synthetic polypeptide
  misc_feature: PD-L1 HC-CDR3
GSYRFWDAF

SEQ ID NO: 269 - protein, 7 aa, Artificial sequence
  Synthetic polypeptide
  misc_feature: PD-L1 HC-CDR3
HSYSSGF

SEQ ID NO: 270 - protein, 9 aa, Artificial sequence
  Synthetic polypeptide
  misc_feature: PD-L1 HC-CDR3
GYFWSYSGF

SEQ ID NO: 271 - protein, 9 aa, Artificial sequence
  Synthetic polypeptide
  misc_feature: PD-L1 HC-CDR3
SDHGVAYGI

SEQ ID NO: 272 - protein, 13 aa, Artificial sequence
  Synthetic polypeptide
  misc_feature: PD-L1 HC-CDR3
GASSDWYFWSSGL

SEQ ID NO: 273 - protein, 7 aa, Artificial sequence
  Synthetic polypeptide
  misc_feature: PD-L1 HC-CDR3
GDWWGAL

SEQ ID NO: 274 - protein, 6 aa, Artificial sequence
  Synthetic polypeptide
  misc_feature: PD-L1 HC-CDR3
YQHLAL

SEQ ID NO: 275 - protein, 16 aa, Artificial sequence
  Synthetic polypeptide
  misc_feature: PD-L1 HC-CDR3
SPSIVWAWHWQYGPGF

SEQ ID NO: 276 - protein, 12 aa, Artificial sequence
  Synthetic polypeptide
  misc_feature: PD-L1 HC-CDR3
SFMYGTWYPYGF

SEQ ID NO: 277 - protein, 18 aa, Artificial sequence
  Synthetic polypeptide
  misc_feature: PD-L1 HC-CDR3
YWWAFHWESHSYQPSYGF

SEQ ID NO: 278 - protein, 12 aa, Artificial sequence
  Synthetic polypeptide
  misc_feature: PD-L1 HC-CDR3
SDFMLHWHWFGM

SEQ ID NO: 279 - protein, 12 aa, Artificial sequence
  Synthetic polypeptide
  misc_feature: PD-L1 HC-CDR3
SHSYGSWYAYGL

SEQ ID NO: 280 - protein, 16 aa, Artificial sequence
  Synthetic polypeptide
  misc_feature: PD-L1 HC-CDR3
WEESRYWYKYYYQGGL

SEQ ID NO: 281 - protein, 8 aa, Artificial sequence
  Synthetic polypeptide
  misc_feature: PD-L1 HC-CDR3
NWSGYLAM

SEQ ID NO: 282 - protein, 12 aa, Artificial sequence
  Synthetic polypeptide
  misc_feature: PD-L1 HC-CDR3
SQSYGSWYAYGL

SEQ ID NO: 283 - protein, 12 aa, Artificial sequence
  Synthetic polypeptide
  misc_feature: PD-L1 HC-CDR3
TSYWEYWYWFGL

SEQ ID NO: 284 - protein, 6 aa, Artificial sequence
  Synthetic polypeptide
  misc_feature: PD-L1 HC-CDR3
HYGFAM

SEQ ID NO: 285 - protein, 18 aa, Artificial sequence
  Synthetic polypeptide
  misc_feature: PD-L1 HC-CDR3
GYYSSYSSWYLYGSDSAI

SEQ ID NO: 286 - protein, 6 aa, Artificial sequence
  Synthetic polypeptide
  misc_feature: PD-L1 HC-CDR3
GYFPAM

SEQ ID NO: 287 - protein, 6 aa, Artificial sequence
  Synthetic polypeptide
  misc_feature: PD-L1 HC-CDR3
HRYFAM

SEQ ID NO: 288 - protein, 15 aa, Artificial sequence
  Synthetic polypeptide
  misc_feature: PD-L1 HC-CDR3
YGGYGEYFSWYPYGM

SEQ ID NO: 289 - protein, 12 aa, Artificial sequence
  Synthetic polypeptide
  misc_feature: PD-L1 HC-CDR3
PYWWSGQGYWGF

SEQ ID NO: 290 - protein, 13 aa, Artificial sequence
  Synthetic polypeptide
  misc_feature: PD-L1 HC-CDR3
GEGQQYKWSPYGL

SEQ ID NO: 291 - protein, 15 aa, Artificial sequence
  Synthetic polypeptide
  misc_feature: PD-L1 HC-CDR3
WYYAWHMGVKGYQGF

SEQ ID NO: 292 - protein, 18 aa, Artificial sequence
  Synthetic polypeptide
  misc_feature: PD-L1 HC-CDR3
QEWYYGFGAYKYHWATGL

SEQ ID NO: 293 - protein, 6 aa, Artificial sequence
  Synthetic polypeptide
  misc_feature: PD-L1 HC-CDR3
WRSLAL

SEQ ID NO: 294 - protein, 13 aa, Artificial sequence
  Synthetic polypeptide
  misc_feature: PD-L1 HC-CDR3
ESYWWWSYWHLGL

SEQ ID NO: 295 - protein, 12 aa, Artificial sequence
  Synthetic polypeptide
  misc_feature: PD-L1 HC-CDR3
GDDHVYWWWFGM

SEQ ID NO: 296 - protein, 8 aa, Artificial sequence
  Synthetic polypeptide
  misc_feature: PD-L1 HC-CDR3
DYSAYYAM

SEQ ID NO: 297 - protein, 6 aa, Artificial sequence
  Synthetic polypeptide
  misc_feature: PD-L1 HC-CDR3
QGWLAL

SEQ ID NO: 298 - protein, 18 aa, Artificial sequence
  Synthetic polypeptide
  misc_feature: PD-L1 HC-CDR3
SYGEGSYTFWIWAGYGAL

SEQ ID NO: 299 - protein, 12 aa, Artificial sequence
  Synthetic polypeptide
  misc_feature: PD-L1 HC-CDR3
SYNYHYYTPYGF

SEQ ID NO: 300 - protein, 12 aa, Artificial sequence
  Synthetic polypeptide
  misc_feature: PD-L1 HC-CDR3
YPYEVSWTPYGM

SEQ ID NO: 301 - protein, 13 aa, Artificial sequence
  Synthetic polypeptide
  misc_feature: PD-L1 HC-CDR3
AEWYLHFEQGFGF

SEQ ID NO: 302 - protein, 8 aa, Artificial sequence
  Synthetic polypeptide
  misc_feature: PD-L1 HC-CDR3
YYYKYMAM

SEQ ID NO: 303 - protein, 18 aa, Artificial sequence
  Synthetic polypeptide
  misc_feature: PD-L1 HC-CDR3
YWWPFHWESHSYQPSYGF

SEQ ID NO: 304 - protein, 17 aa, Artificial sequence
  Synthetic polypeptide
  misc_feature: PD-L1 HC-CDR3
SYNYQGDNWHEYYPSGL

SEQ ID NO: 305 - protein, 6 aa, Artificial sequence
  Synthetic polypeptide
  misc_feature: PD-L1 HC-CDR3
YYGYGM

SEQ ID NO: 306 - protein, 7 aa, Artificial sequence
  Synthetic polypeptide
  misc_feature: RNF43 HC-CDR1
AFSYYDY

SEQ ID NO: 307 - protein, 7 aa, Artificial sequence
  Synthetic polypeptide
  misc_feature: RNF43 HC-CDR1
DFYSSDD

SEQ ID NO: 308 - protein, 8 aa, Artificial sequence
  Synthetic polypeptide
  misc_feature: RNF43 HC-CDR1
AIYSYYYD

SEQ ID NO: 309 - protein, 7 aa, Artificial sequence
  Synthetic polypeptide
  misc_feature: RNF43 HC-CDR1
AIYYSYD

SEQ ID NO: 310 - protein, 8 aa, Artificial sequence
  Synthetic polypeptide
  misc_feature: RNF43 HC-CDR1
RISSYYYD

SEQ ID NO: 311 - protein, 10 aa, Artificial sequence
  Synthetic polypeptide
  misc_feature: RNF43 HC-CDR2
RIYPYSSYTS

SEQ ID NO: 312 - protein, 10 aa, Artificial sequence
  Synthetic polypeptide
  misc_feature: RNF43 HC-CDR2
RIYSSYGSTY

SEQ ID NO: 313 - protein, 10 aa, Artificial sequence
  Synthetic polypeptide
  misc_feature: RNF43 HC-CDR2
RIYPYSGYTS

SEQ ID NO: 314 - protein, 14 aa, Artificial sequence
  Synthetic polypeptide
  misc_feature: RNF43 HC-CDR3
YSPGYYPFRGWGGM

SEQ ID NO: 315 - protein, 14 aa, Artificial sequence
  Synthetic polypeptide
  misc_feature: RNF43 HC-CDR3
YAWRPSGGYYSYAM

SEQ ID NO: 316 - protein, 9 aa, Artificial sequence
  Synthetic polypeptide
  misc_feature: RNF43 HC-CDR3
GSYYFGYAF

SEQ ID NO: 317 - protein, 14 aa, Artificial sequence
  Synthetic polypeptide
  misc_feature: RNF43 HC-CDR3
WGWSYDPAGYAYAM

SEQ ID NO: 318 - protein, 7 aa, Artificial sequence
  Synthetic polypeptide
  misc_feature: RNF43 HC-CDR3
WYDSPGF

SEQ ID NO: 319 - protein, 208 aa, Artificial sequence
  Synthetic polypeptide
  misc_feature: Alternative IgG light chain framework regions
  misc_feature (90)..(91): LC-CDR3
  misc_feature (102)..(208): constant domain
DIQMTQSPSSLSASVGDRVTITCRASQSVGSALAWYQQKPGKAPKLLIYSASSLYSGVPSRFSGSRSGTDFTLTISSLQPEDFATYYCQQTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 320 - protein, 198 aa, Artificial sequence
  Synthetic polypeptide
  misc_feature: Alternative IgG heavy chain Fab framework regions
  misc_feature (28)..(29): HC-CDR1
  misc_feature (43)..(44): HC-CDR2
  misc_feature (82)..(83): HC-CDR3
  misc_feature (96)..(198): HC-CDR3
EVQLVESGGGLVQPGGSLRLSCAASGFNHWVRQAPGKGLEWVAYADSVKGRFTISADTSKNTAYLQMNSLRAEDTAVYYCARDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 321 - protein, 95 aa, Artificial sequence
  Synthetic polypeptide
  misc_feature: VH binder framework regions
  misc_feature (27)..(28): HC-CDR1
  misc_feature (43)..(44): HC-CDR2
  misc_feature (82)..(83): HC-CDR3
EVQLVESGGGLVQPGGSLRLSCAASGFIGWVRRAPGKGEELVAYADSVKGRFTISADTSKNTAYLQMNSLRAEDTAVYYCARDYWGQGTLVTVSS

SEQ ID NO: 322 - protein, 213 aa, Artificial sequence
  Synthetic polypeptide
  misc_feature: RNF43 A4 Light Chain Construct
  misc_feature (91)..(95): LC-CDR3 region
  misc_feature (107)..(213): constant domain
DIQMTQSPSSLSASVGDRVTITCRASQSVGSALAWYQQKPGKAPKLLIYSASSLYSGVPSRFSGSRSGTDFTLTISSLQPEDFATYYCQQVYPPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 323 - protein, 235 aa, Artificial sequence
  Synthetic polypeptide
  misc_feature: RNF43 A4 Heavy Chain Construct
  misc_feature (32)..(37): CDR region
  misc_feature (53)..(62): CDR region
  misc_feature (102)..(119): CDR region
  misc_feature (133)..(235): constant domain
EISEVQLVESGGGLVQPGGSLRLSCAASGFNLSYSYIHWVRQAPGKGLEWVASISPSYGYTYYADSVKGRFTISADTSKNTAYLQMNSLRAEDTAVYYCARPYHPFGGHYWWPYYYHGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 324 - protein, 216 aa, Artificial sequence
  Synthetic polypeptide
  misc_feature: RNF43 A6 Light Chain Construct
  misc_feature (91)..(98): LC CDR3 region
  misc_feature (110)..(216): constant domain
DIQMTQSPSSLSASVGDRVTITCRASQSVGSALAWYQQKPGKAPKLLIYSASSLYSGVPSRFSGSRSGTDFTLTISSLQPEDFATYYCQQSKYSNQLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 325 - protein, 223 aa, Artificial sequence
  Synthetic polypeptide
  misc_feature: RNF43 A6 Heavy Chain Construct
  misc_feature (32)..(37): CDR region
  misc_feature (53)..(62): CDR region
  misc_feature (102)..(107): CDR region
  misc_feature (121)..(223): constant domain
EISEVQLVESGGGLVQPGGSLRLSCAASGFNVSYYYIHWVRQAPGKGLEWVASIYSSYGSTYYADSVKGRFTISADTSKNTAYLQMNSLRAEDTAVYYCAREYYFGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 326 - protein, 212 aa, Artificial sequence
  Synthetic polypeptide
  misc_feature: anti-RNF43 Fab LC in the conjugate
DIQLTQMPSSLSASVGDRVTITCRASQSVGSALAWYQQKPGKAPKLLIYSASSLYSGVPSRFSGSRSGTDFTLTISSLQPEDFATYYCQQAYPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 327 - protein, 258 aa, Artificial sequence
  Synthetic polypeptide
  misc_feature: anti-RNF43 Fab HC in the conjugate
EISEVQLVESGGGLVQPGGSLRLSCAASGFNIYYYSLHWVRQAPGKGLEWVASISPYYSYTSYADSVKGRFTISADTSKNTAYLQLNSLRAEDTAVYYCARYGYYGWDYHRYSAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGSGSAGGLNDIFEAQKIEWHE

SEQ ID NO: 328 - protein, 213 aa, Artificial sequence
  Synthetic polypeptide
  misc_feature: Anti-MMP14 Fab HC-binder 1
  misc_feature (31)..(35): CDR region
  misc_feature (50)..(66): CDR region
  misc_feature (99)..(104): CDR region
EVQLLESGGGLVQPGGSLRLSCAASGFTFSLYSMNWVRQAPGKGLEWVSSIYSSGGSTLYADSVKGRFTISRDNSKNTLYLQMNSLRAEDTAVYYCARGRAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 329 - protein, 215 aa, Artificial sequence
  Synthetic polypeptide
  misc_feature: Anti-MMP14 Fab LC-binder 1
  misc_feature (24)..(34): CDR region
  misc_feature (50)..(60): CDR region
  misc_feature (89)..(98): CDR region
DIQMTQSPSSLSASVGDRVTITCRASQSVGTYLNWYQQKPGKAPKLLIYATSNLRSGVPSRFSGSGSGTDFTLTISSLQPEDFATYYCQQSYSIPRFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 330 - protein, 234 aa, Artificial sequence
  Synthetic polypeptide
  misc_feature: Anti-MMP14 Fab HC-binder 2
  misc_feature (29)..(34): CDR region
  misc_feature (50)..(59): CDR region
  misc_feature (99)..(125): CDR region
EVQLVESGGGLVQPGGSLRLSCAASGFNLSSSSMHWVRQAPGKGLEWVASIYPSYSYTYYADSVKGRFTISADTSKNTAYLQMNSLRAEDTAVYYCARVKLQKDKSHQWIRNLVATPYGRYVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 331 - protein, 213 aa, Artificial sequence
  Synthetic polypeptide
  misc_feature: Anti-MMP14 Fab LC-binder 2
  misc_feature (24)..(34): CDR region
  misc_feature (50)..(56): CDR region
  misc_feature (89)..(96): CDR region
DIQMTQSPSSLSASVGDRVTITCRASQSVSSAVAWYQQKPGKAPKLLIYSASSLYSGVPSRFSGSRSGTDFTLTISSLQPEDFATYYCQQYGYPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 332 - protein, 212 aa, Artificial sequence
  Synthetic polypeptide
  misc_feature: RNF43 A5 Light Chain Construct
  misc_feature (91)..(94): LC CDR3 region
  misc_feature (106)..(212): constant domain
DIQMTQSPSSLSASVGDRVTITCRASQSVGSALAWYQQKPGKAPKLLIYSASSLYSGVPSRFSGSRSGTDFTLTISSLQPEDFATYYCQQAYPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 333 - protein, 231 aa, Artificial sequence
  Synthetic polypeptide
  misc_feature: RNF43 A5 Heavy Chain Construct
  misc_feature (32)..(37): CDR region
  misc_feature (53)..(62): CDR region
  misc_feature (102)..(115): CDR region
  misc_feature (129)..(231): constant region
EISEVQLVESGGGLVQPGGSLRLSCAASGFNIYYYSMHWVRQAPGKGLEWVASISPYYSYTSYADSVKGRFTISADTSKNTAYLQMNSLRAEDTAVYYCARYGYYGWDYHRYSAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 334 - protein, 107 aa, Artificial sequence
  Synthetic polypeptide
  misc_feature: Constant domain CL1 version 2
RTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 335 - protein, 232 aa, Artificial sequence
  Synthetic polypeptide
  misc_feature: CH2-CH3 (Knob construct with N297G)
EPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 336 - protein, 232 aa, Artificial sequence
  Synthetic polypeptide
  misc_feature: CH2-CH3 (Hole construct with N297G)
EPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 337 - protein, 232 aa, Artificial sequence
  Synthetic polypeptide
  misc_feature: CH2-CH3 (WT sequence)
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 338 - protein, 89 aa, Artificial sequence
  Synthetic polypeptide
  misc_feature: IgG light chain framework region 1
DIQMTQSPSSLSASVGDRVTITCRASQSVSSAVAWYQQKPGKAPKLLIYSASSLYSGVSRFSGSRSGTDFTLTISSLQPEDFATYYCQQ

SEQ ID NO: 339 - protein, 118 aa, Artificial sequence
  Synthetic polypeptide
  misc_feature: IgG light chain framework region 2
TFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 340 - protein, 30 aa, Artificial sequence
  Synthetic polypeptide
  misc_feature: IgG heavy chain Fab framework region 1
EISEVQLVESGGGLVQPGGSLRLSCAASGF

SEQ ID NO: 341 - protein, 13 aa, Artificial sequence
  Synthetic polypeptide
  misc_feature: IgG heavy chain Fab framework region 2
WVRQAPGKGLEWV

SEQ ID NO: 342 - protein, 39 aa, Artificial sequence
  Synthetic polypeptide
  misc_feature: IgG heavy chain Fab framework region 3
YADSVKGRFTISADTSKNTAYLQMNSLRAEDTAVYYCAR

SEQ ID NO: 343 - protein, 116 aa, Artificial sequence
  Synthetic polypeptide
  misc_feature: IgG heavy chain Fab framework region 4
DYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 344 - protein, 90 aa, Artificial sequence
  Synthetic polypeptide
  misc_feature: Alternative IgG light chain framework region 1
DIQMTQSPSSLSASVGDRVTITCRASQSVGSALAWYQQKPGKAPKLLIYSASSLYSGVPSRFSGSRSGTDFTLTISSLQPEDFATYYCQQ

SEQ ID NO: 345 - protein, 118 aa, Artificial sequence; identical to SEQ ID NO: 339
  Synthetic polypeptide
  misc_feature: Alternative IgG light chain framework region 2

SEQ ID NO: 346 - protein, 28 aa, Artificial sequence
  Synthetic polypeptide
  misc_feature: Alternative IgG heavy chain Fab framework region 1
EVQLVESGGGLVQPGGSLRLSCAASGFN

SEQ ID NO: 347 - protein, 15 aa, Artificial sequence
  Synthetic polypeptide
  misc_feature: Alternative IgG heavy chain Fab framework region 2
HWVRQAPGKGLEWVA

SEQ ID NO: 348 - protein, 39 aa, Artificial sequence; identical to SEQ ID NO: 342
  Synthetic polypeptide
  misc_feature: Alternative IgG heavy chain Fab framework region 3

SEQ ID NO: 349 - protein, 116 aa, Artificial sequence; identical to SEQ ID NO: 343
  Synthetic polypeptide
  misc_feature: Alternative IgG heavy chain Fab framework region 4

SEQ ID NO: 350 - protein, 27 aa, Artificial sequence
  Synthetic polypeptide
  misc_feature: VH binder framework region 1
EVQLVESGGGLVQPGGSLRLSCAASGF

SEQ ID NO: 351 - protein, 16 aa, Artificial sequence
  Synthetic polypeptide
  misc_feature: VH binder framework region 2
IGWVRRAPGKGEELVA

SEQ ID NO: 352 - protein, 39 aa, Artificial sequence; identical to SEQ ID NO: 342
  Synthetic polypeptide
  misc_feature: VH binder framework region 3

SEQ ID NO: 353 - protein, 13 aa, Artificial sequence
  Synthetic polypeptide
  misc_feature: VH binder framework region 4
DYWGQGTLVTVSS

SEQ ID NO: 354 - protein, 1272 aa, Artificial sequence
  Synthetic polypeptide
  misc_feature: RNF43-antiGFP scFab construct alternative
SDIQMTQSPSSLSASVGDRVTITCRASQSVSSAVAWYQQKPGKAPKLLIYSASSLYSGVPSRFSGSRSGTDFTLTISSLQPEDFATYYCQQSWGLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGECGGSSGSGSGSTGTSSSGTGTSAGTTGTSASTSGSGSGGGGGSGGGGSAGGTATAGASSGSEVQLVESGGGLVQPGGSLRLSCAASGFNISYYSIHWVRQAPGKGLEWVASIYPYYSSTSYADSVKGRFTISADTSKNTAYLQMNSLRAEDTAVYYCARAGWVASSGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGFGRTGLVLAAAVESERSAEQKAIIRVIPLKMDPTGKLNLTLEGVFAGVAEITPAEGKLMQSHPLYLCNASDDDNLEPGFISIVKLESPRRAPRPCLSLASKARMAGERGASAVLFDITEDRAAAEQLQQPLGLTWPVVLIWGNDAEKLMEFVYKNQKAHVRIELKEPPAWPDYDVWILMTVVGTIFVIILASVLRIRCRPRHSRPDPLQQRTAWAISQLATRRYQASCRQARGEWPDSGSSCSSAPVCAICLEEFSEGQELRVISCLHEFHRNCVDPWLHQHRTCPLCMFNITEGDSFSQSLGPSRSYQEPGRRLHLIRQHPGHAHYHLPAAYLLGPSRSAVARPPRPGPFLPSQEPGMGPRHHRFPRAAHPRAPGEQQRLAGAQHPYAQGWGLSHLQSTSQHPAACPVPLRRARPPDSSGSGESYCTERSGYLADGPASDSSSGPCHGSSSDSVVNCTDISLQGVHGSSSTFCSSLSSDFDPLVYCSPKGDPQRVDMQPSVTSRPRSLDSVVPTGETQVSSHVHYHRHRHHHYKKRFQWHGRKPGPETGVPQSRPPIPRTQPQPEPPSPDQQVTRSNSAAPSGRLSNPQCPRALPEPAPGPVDASSICPSTSSLFNLQKSSLSARHPQRKRRGGPSEPTPGSRPQDATVHPACQIFPHYTPSVAYPWSPEAHPLICGPPGLDKRLLPETPGPCYSNSQPVWLCLTPRQPLEPHPPGEGPSEWSSDTAEGRPCPYPHCQVLSAQPGSEEELEELCEQAV

SEQ ID NO: 355 - protein, 214 aa, Artificial sequence
  Synthetic polypeptide
  misc_feature: anti-GFP Light chain domain of SEQ ID NO354
SDIQMTQSPSSLSASVGDRVTITCRASQSVSSAVAWYQQKPGKAPKLLIYSASSLYSGVPSRFSGSRSGTDFTLTISSLQPEDFATYYCQQSWGLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 356 - protein, 208 aa, Artificial sequence
  Synthetic polypeptide
  misc_feature: IgG light chain framework regions, alternative
  misc_feature (90)..(91): LC-CDR3
  misc_feature (102)..(208): LC-CDR3
SDIQMTQSPSSLSASVGDRVTITCRASQSVSSAVAWYQQKPGKAPKLLIYSASSLYSGVSRFSGSRSGTDFTLTISSLQPEDFATYYCQQTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 357 - protein, 466 aa, Artificial sequence
  Synthetic polypeptide
  misc_feature: Bispecific IgG having a single chain Fab as one arm, alternative
  misc_feature (90)..(91): LC-CDR3
  misc_feature (102)..(208): constant domain
  misc_feature (298)..(299): HC-CDR1
  misc_feature (311)..(312): HC-CDR2
  misc_feature (350)..(351): HC-CDR3
  misc_feature (364)..(466): constant domain
SDIQMTQSPSSLSASVGDRVTITCRASQSVSSAVAWYQQKPGKAPKLLIYSASSLYSGVSRFSGSRSGTDFTLTISSLQPEDFATYYCQQTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGECGGSSGSGSGSTGTSSSGTGTSAGTTGTSASTSGSGSGGGGGSGGGGSAGGTATAGASSGSEISEVQLVESGGGLVQPGGSLRLSCAASGFWVRQAPGKGLEWVYADSVKGRFTISADTSKNTAYLQMNSLRAEDTAVYYCARDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 358 - protein, 208 aa, Artificial sequence; identical to SEQ ID NO: 356
  Synthetic polypeptide
  misc_feature: Fab-scFv construct - heavy chain-scFv fusion, alternative (light chain)
  misc_feature (90)..(91): Antigen1-LC-CDR3
  misc_feature (102)..(208): constant domain

SEQ ID NO: 359 - protein, 111 aa, Artificial sequence
  Synthetic polypeptide
  misc_feature: LC variable domain, anti-CDCP1; alternative
AASDIQMTQSPSSLSASVGDRVTITCRASQSVSSAVAWYQQKPGKAPKLLIYSASSLYSGVPSRFSGSRSGTDFTLTISSLQPEDFATYYCQQSYYYYPITFGQGTKVEIK

SEQ ID NO: 360 - protein, 123 aa, Artificial sequence
  Synthetic polypeptide
  misc_feature: HC variable domain, anti-CDCP1; alternative
AAQPEISEVQLVESGGGLVQPGGSLRLSCAASGFNLSYYYIHWVRQAPGKGLEWVASIYSSSSYTSYADSVKGRFTISADTSKNTAYLQMNSLRAEDTAVYYCARAYYGFDYWGQGTLVTVSS